(19) United States
(12) Patent Application Publication
(10) Pub. No.: US 2023/0346936 A1
(43) Pub. Date: Nov. 2, 2023

(54) EX VIVO EXPANSION OF REGULATORY T CELLS FOR SUPPRESSION OF GRAFT VERSUS HOST DISEASE

(71) Applicant: City of Hope, Duarte, CA (US)

(72) Inventor: Fouad Kandeel, Duarte, CA (US)

(21) Appl. No.: 18/026,335

(22) PCT Filed: Sep. 15, 2021
(86) PCT No.: PCT/US2021/050515

§ 371 Date: Mar. 14, 2023

Related U.S. Application Data
(60) Provisional application No. 63/078,740, filed on Sep. 15, 2020.

(51) Int. Cl. A61K 39/00; C12N 5/0783; A61P 37/06

(52) U.S. Cl. CPC A61K 39/4621; C12N 5/0637; A61K 39/4611; A61K 39/4615; A61K 39/4612; A61P 37/06; C12N 2501/2302; C12N 2501/51; C12N 2501/515; C12N 2502/1107; C12N 2502/1121

(57) ABSTRACT

Provided herein, inter alia, are methods and compositions for treating or preventing graft-versus-host disease. The methods include administering to a tissue transplant recipient a composition comprising a donor-derived regulatory T cell.

### CROSS-REFERENCE TO RELATED APPLICATIONS

This application claims priority to U.S. Provisional Application No. 63/078,740, filed Sep. 15, 2020, which is hereby incorporated by reference in its entirety and for all purposes.

### BACKGROUND

Graft-versus-host disease (GvHD) is a medical complication following the receipt of transplanted tissue from a genetically different person. GvHD is commonly associated with stem cell transplants such as those that occur with bone marrow transplants. GvHD also applies to other forms of transplanted tissues such as solid organ transplants.

White blood cells of the donor's immune system, which remain within the donated tissue (the graft) recognize the recipient (the host) as foreign (non-self). The white blood cells present within the transplanted tissue then attack the recipient's body's cells, which leads to GvHD. This should not be confused with a transplant rejection, which occurs when the immune system of the transplant recipient rejects the transplanted tissue; GvHD occurs when the donor's white blood cells reject the recipient. The underlying principle (alloimmunity) is the same, but the details and course may differ. GvHD can also occur after a blood transfusion if the blood products used have not been irradiated or treated with an approved pathogen reduction system.

The pathophysiology of GvHD includes three phases: 1) Activation of APC (antigen presenting cells); 2) Activation, proliferation, differentiation and migration of effector cells; 3) Target tissue destruction. Activation of APC occurs in the first stage of GvHD. Prior to hematopoietic stem cell transplantation, radiation or chemotherapy results in damage of host tissues, especially intestinal mucosa. This allows the microbial products to enter and stimulate pro-inflammatory cytokines such as IL-1 and TNF-α. These proinflammatory cytokines increase the expression of MHC and adhesion molecules on APCs, thereby increasing the ability of APC to present antigen. The second phase is characterized by the activation of effector cells. Activation of donor T-cells further enhances the expression of MHC and adhesion molecules, chemokines and the expansion of CD8+ and CD4+ T-cells and guest B-cells. In the final phase, these effector cells migrate to target organs and mediate tissue damage, resulting in multi-organ failure.

Immunosuppressants are powerful drugs used in various aspects of organ transplant but are capable of triggering severe adverse effects. Hence, there is tremendous interest in replacing them with less-toxic agents. Adoptive therapy with CD25+CD4+ T regulatory cells (Treg cells) holds promise as an alternative to immunosuppressants. Treg cells have been described as the most potent immunosuppressive cells in the human body. In a number of experimental models, they have been found to quench autoimmune diseases, maintain allogeneic transplants, and prevent allergic diseases.

A major stumbling block in their clinical application has been related to Treg phenotype and the very limited number of these cells in the periphery, not exceeding 5-10% of total CD4+ T cells. Recent progress in multicolor flow cytometry and cell sorting as well as cellular immunology has found ways of overcoming these obstacles, and has opened the doors to the clinical application of Treg cells, Treg adoptive therapy is a promising tool in immunosuppressive therapy, as it has been found to effectively quench autoimmune and allergic reactions and increase tolerance after allo-transplantations. The development of multicolor flow cytometry and cell sorting made the expansion of Treg cells possible. It is of special importance in human immunology as pure sorting of these cells requires multicolor staining with sequential gating that involves gates based on differences in the expression levels of Treg markers. Reasonable purity and viability of sorted Treg cells for ex vivo expansion was achieved only recently, with the last generation of sorting equipment.

Previous studies suggest that infusing patients with regulatory T cells (Treg cells) could form a promising cellular therapy for halting immune-mediated diseases including GvHD. However, each GvHD patient has their own antigen. There remains a need for efficient methods to generate and expand antigen-specific Treg cells (aTreg cells) for the treatment of immune-mediated diseases such as GvHD.

### BRIEF SUMMARY OF THE INVENTION

In an aspect is provided a method of treating or preventing graft-versus-host disease in a subject in need thereof, the method including administering to the subject a therapeutically effective amount of recipient antigen-specific regulatory T cells, thereby treating or preventing graft-versus-host disease in the subject; wherein the recipient antigen-specific regulatory T cells are derived from regulatory T cells from a donor of a tissue transplant to the subject; and wherein the tissue transplant is a cause of graft-versus-host disease in the subject.

In an aspect, the recipient antigen-specific regulatory T cells provided herein including embodiments thereof are formed by a method including: (a) expanding the regulatory T cells in vitro, thereby forming a plurality of regulatory T cells; and (b) contacting the plurality of regulatory T cells with a plurality of recipient antigen presenting cells and a CD28 inhibitor compound in vitro, thereby forming the recipient antigen-specific regulatory T cells.

In an aspect, the recipient antigen-specific regulatory T cells provided herein including embodiments thereof are formed by a method including: (a) expanding regulatory T cells in vitro, thereby forming a plurality of regulatory T cells; (b) contacting a plurality of recipient antigen presenting cells with recipient antigen in vitro, thereby forming a plurality of activated recipient antigen presenting cells; and (c) contacting the plurality of regulatory T cells with the plurality of activated recipient antigen presenting cells in the presence of a CD28 inhibitor compound, thereby forming the recipient antigen-specific regulatory T cells.

In an aspect is provided a method of forming recipient antigen-specific regulatory T cells, the method including: (a) expanding regulatory T cells in vitro, wherein the regulatory T cells are from a donor of a tissue transplant, thereby forming a plurality of regulatory T cells; and (b) contacting the plurality of regulatory T cells with a plurality of recipient antigen presenting cells and a CD28 inhibitor compound in vitro, wherein the plurality of recipient antigen presenting cells is from a subject who has received the tissue transplant, thereby forming the recipient antigen-specific regulatory T cells.

In another aspect is provided a method of forming recipient antigen-specific regulatory T cells, the method including: (a) expanding regulatory T cells in vitro, wherein the regulatory T cells are from a donor of a tissue transplant, thereby forming a plurality of regulatory T cells; (b) contacting a plurality of recipient antigen presenting cells with a recipient antigen in vitro, wherein the plurality of recipient antigen presenting cells is from a subject who has received the tissue transplant, thereby forming a plurality of activated recipient antigen presenting cells; and (c) contacting the plurality of regulatory T cells with the plurality of activated recipient antigen presenting cells and a CD28 inhibitor compound in vitro, thereby forming the recipient antigen-specific regulatory T cells.

In an aspect is provided an antigen-specific regulatory T cell derived from a donor of a transplant tissue, wherein the antigen-specific regulatory T cell specifically includes a T-cell receptor that specifically binds a transplant tissue antigen from a recipient of the transplant tissue.

In another aspect a pharmaceutical composition including recipient antigen-specific regulatory T cells is provided, wherein the recipient antigen-specific regulatory T cells are formed by a method provided herein including embodiments thereof.

In an aspect is provided a method of treating or preventing graft-versus-host disease in a subject in need thereof, the method including administering to the subject a therapeutically effective amount of a donor-sourced regulatory T cell; thereby treating or preventing graft-versus-host disease in the subject.

In an aspect is provided a method of forming a donor-sourced regulatory T cell, the method including expanding a regulatory T cell in vitro, thereby forming an expanded regulatory T cell; and contacting the expanded regulatory T cell with an antigen presenting cell and a CD28 inhibitor compound (e.g. belatacept) in vitro, thereby forming anergic recipient antigen-specific regulatory T cells.

In an aspect is provided a method of forming an anergic recipient antigen-specific regulatory T cell, the method comprising expanding donor-sourced regulatory T cell in vitro, contacting the expanded donor-sourced regulatory T cell with the activated recipient-derived antigen presenting cell; thereby forming the recipient antigen-specific regulatory T cell.

In an aspect is provided a method the expansion of donor-sourced antigen-specific Treg cells by anti-CD28 costimulatory blockade and APC stimulation to treat recipient GvHD patients. Exposing the expanding Treg cells to antigens on the surface of the antigen presenting cells from recipient while costimulation through the CD28 receptor is blocked renders the Treg cells anergic towards presented antigens.

### BRIEF DESCRIPTION OF THE DRAWINGS

FIG. 1 is a flow chart of an example experimental design for generating antigen-specific Treg from GvHD patients by Belatacept treatment and APC stimulation.

FIG. 2 is the FACS results showing the phenotypic analysis of PBMC from a donor before the selection process has taken place.

FIG. 3 is the FACS results showing the phenotypic analysis of PBMC from a patient before the selection process has taken place.

FIG. 4 is the FACS results showing the phenotypic analysis of PBMC from a patient for CD1c+ or CD303+ and CD141+ before selection process has taken place.

FIG. 5 is the FACS results showing the phenotypic analysis of dendritic cells after selection process has taken place.

FIG. 6 are results of Donor nTreg after isolation before and after sorting.

FIG. 7 are results of Donor aTreg after isolation before and after sorting.

FIG. 8 is the FoxP3 expression analysis of CD4+ T cells from a donor at different time points: before co-culturing, after co-culturing with CTLA-4-Ig and APCs, and after further stimulated with APCs.

FIG. 9 is a table showing the fold expansion of different Treg populations as activated by T cell activator.

FIG. 10 is the FACS analysis of Foxp3 expression of nTreg cells and aTreg cells after expansion on day 16.

FIG. 11 is the data showing comparison of Treg function between nTreg and aTreg on D16.

FIG. 12 is the data showing comparison of Foxp3 TSDR of nTreg and aTreg on D16.

FIG. 13 is data showing the cytokine profile of different Treg cells on D16.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein in other proteins with different numbering systems. For example, by performing a simple sequence alignment with a protein the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is to correspond to the glutamic acid 138 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “CD28” or “CD28 protein” as referred to herein includes any of the recombinant or naturally-occurring forms of the T-cell-specific surface glycoprotein CD28 (CD28) protein, also known as TP44, cluster of differentiation 28, or variants or homologs thereof (including functional fragments thereof) that maintain CD28 protein activity (e.g. within at least 50%, 80%, 90%, 95%, 96%, 97%, 98%, 99% or 100% activity compared to CD28 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 protein. In embodiments, the CD28 protein is substantially identical to the protein identified by the UniProt reference number P10747 or a variant or homolog having substantial identity thereto.

The term “CD80” or “CD80 protein” as referred to herein includes any of the recombinant or naturally-occurring forms of the T-lymphocyte activation antigen CD80 (CD80) protein, also known as Activation B7-1 antigen, BB1, CTLA-4 counter-receptor B7.1, cluster of differentiation 80, or variants or homologs thereof (including functional fragments thereof) that maintain CD80 protein activity (e.g. within at least 50%, 80%, 90%, 95%, 96%, 97%, 98%, 99% or 100% activity compared to CD80 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD80 protein. In embodiments, the CD80 protein is substantially identical to the protein identified by the UniProt reference number P33681 or a variant or homolog having substantial identity thereto.

The term “human leukocyte antigen II” or “human leukocyte antigen II protein” as used herein refers to any of the recombinant or naturally-occurring forms of human leukocyte antigen II (HLA II) also known as MHC class II human leukocyte antigen or human leukocyte antigen class II, or variants or homologs thereof (including functional fragments thereof) that maintain human leukocyte antigen II activity (e.g., within at least 50%, 80%, 90%, 95%, 96%, 97%, 98%, 99% or 100% activity compared to Human Leukocyte Antigen II). In aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring human leukocyte antigen II. In aspects, the human leukocyte antigen II protein is substantially identical to the protein identified by the NCBI reference number GI: 122206, or a variant or homolog having substantial identity thereto. In aspects, the human leukocyte antigen II protein is substantially identical to the protein identified by the NCBI reference number GI: 451344622, or a variant or homolog having substantial identity thereto. In aspects, the human leukocyte antigen II protein is substantially identical to the protein identified by the NCBI reference number GI: 290457643, or a variant or homolog having substantial identity thereto. In embodiments, the human leukocyte antigen II protein is substantially identical to the protein identified by the NCBI reference number GI: 545422, or a variant or homolog having substantial identity thereto.

The term “CD86” or “CD86 protein” as referred to herein includes any of the recombinant or naturally-occurring forms of the T-lymphocyte activation antigen CD86 (CD86) protein, also known as Activation B7-2 antigen, B70, CTLA-4 counter-receptor B7.2, cluster of differentiation 86, or variants or homologs thereof (including functional fragments thereof) that maintain CD86 protein activity (e.g. within at least 50%, 80%, 90%, 95%, 96%, 97%, 98%, 99% or 100% activity compared to CD86 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD86 protein. In embodiments, the CD86 protein is substantially identical to the protein identified by the UniProt reference number P42081 or a variant or homolog having substantial identity thereto.

A “Forkhead box protein P3” or “FoxP3” as referred to herein includes any of the recombinant or naturally-occurring forms of the Forkhead box protein P3 (FoxP3) also known as scurfin, or variants or homologs thereof (including functional fragments thereof) that maintain FoxP3 protein activity (e.g. within at least 50%, 80%, 90%, 95%, 96%, 97%, 98%, 99% or 100% activity compared to FoxP3 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring FoxP3 protein. In embodiments, the FoxP3 protein is substantially identical to the protein identified by the UniProt reference number Q9BZS1 or a variant or homolog having substantial identity thereto.

Antibodies are large, complex molecules (molecular weight of 150,000 Da or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site (paratope), which docks onto the target antigen (epitope). The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially a Fab with part of the hinge region (see Fundamental Immunology (Paul ed., 3rd ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term “antigen” as provided herein refers to a molecule (e.g. a protein) present in and/or produced by a subject (e.g. recipient) who has received a tissue transplant, or a molecule present in and/or produced by the donor of the tissue transplant. An antigen is capable of producing an immune response (e.g. an immune response that may contribute or be a cause of graft versus host disease in said subject). For example, the subject may express one or more proteins (e.g. recipient antigens), which are not present in the donor. In embodiments, the antigen present in and/or expressed by the subject may be a variant of a protein not present in and/or expressed by the donor (of a tissue donated to the subject/recipient). For example, the antigen present in and/or expressed by the subject (e.g. recipient antigen) may be a tissue antigen. In embodiments, the recipient antigen may be presented by a class-I major H complex (MHC) antigen or a class-II MHC antigens not present in and/or expressed by the donor. Thus, in embodiments, the recipient antigen is considered to be foreign to the donor. Similarly, the donor of the tissue transplant may express one or more antigens which are not present in the subject (e.g. recipient).

As used herein, the term “contacting” is used in accordance with its plain ordinary meaning and refers to the process of allowing at least two distinct species (e.g. an antigen and antigen presenting cell (APC), an APC and Treg cell, etc.) to become sufficiently proximal to react, interact or physically touch. It should be appreciated, however, that the resulting reaction product can be produced directly from a reaction between the added reagents or from an intermediate from one or more of the added reagents, which can be produced in the reaction mixture. The term “contacting” may include allowing two species to react, interact, or physically touch, wherein the two species may be a cell (e.g. Treg cell) as provided herein and another cell (e.g. APC) provided. In embodiments contacting includes allowing an agent provided herein to interact with a cell. In embodiments contacting includes allowing an agent provided herein (e.g. a CD28 inhibitor compound) to interact with an immune cell (e.g. Treg cell). In embodiments, contacting includes a recipient antigen interacting with an APC. In embodiments, contacting includes a Treg cell from a tissue transplant donor interacting with an APC from a transplant recipient (e.g. recipient APC). In embodiments, contacting includes allowing a cell to become sufficiently proximal to another cell in a cell culture.

The term “contacting” may include allowing two species to react, interact, or physically touch, wherein the two species may be, for example, a pharmaceutical composition (e.g. recipient antigen-specific regulatory T cell) as provided herein and a cell. In embodiments contacting includes, for example, allowing a pharmaceutical composition as described herein to interact with a cell.

The terms “bind” and “bound” as used herein is used in accordance with its plain and ordinary meaning and refers to the association between atoms or molecules. The association can be covalent (e.g., by a covalent bond or linker) or non-covalent (e.g., electrostatic interactions (e.g., ionic bond, hydrogen bond, or halogen bond), van der Waals interactions (e.g., dipole-dipole, dipole-induced dipole, or London dispersion), ring stacking (pi effects), hydrophobic interactions, and the like). For example, a cell surface receptor on an immune cell may bind to a ligand though non-covalent interactions.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In embodiments, the biological sample is blood. In embodiments, the biological sample is bone marrow. In embodiments, the biological sample includes a hematopoietic stem cell.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

A “stem cell” is a cell characterized by the ability of self-renewal through mitotic cell division and the potential to differentiate into a tissue or an organ. Mammalian stem cells include embryonic stem cells (ES cells) and somatic stem cells. “Hematopoietic stem cells” or “HSCs”, also referred to as blood stem cells, are characterized by the ability to develop into blood cells, which may include white blood cells, red blood cells, and platelets. HSCs can be found in the peripheral blood and bone marrow. HSCs may be used to treat diseases including but not limited to leukemia, lymphoma, and sickle cell disease. For example, HSCs may be used to replace or replenish a subject's hematopoietic system.

The term “antigen presenting cell” or “APC” is used in accordance with its plain and ordinary meaning and refers to a heterogeneous group of immune cells that mediate the cellular immune response by processing and presenting antigens for recognition by certain lymphocytes, for example T cells. The antigens are typically complexed with major histocompatibility complexes (MHCs) on the APC surface for display. APCs include dendritic cells, macrophages, Langerhans cells and B cells. DCs are the most potent professional APCs known to elicit primary T cell responses. In embodiments, the APC is an APC from the recipient of a tissue transplant. Thus, in embodiments, the recipient APC presents antigens from the recipient. In embodiments, an APC is a dentritic cell. In embodiments, an APC is a B cell. In embodiments, APCs include a plurality of dentritic cells and B cells.

As used herein, the term “dendritic cell” or “DC” is used in accordance with its plain and ordinary meaning and refers to an antigen-presenting cell of the immune system. Dendritic cells process antigens and in embodiments present the processed antigen on the cell surface. In embodiments, presentation of processed antigen results in activation and/or proliferation of T cells (e.g. helper T-cells, killer T cells). DCs include conventional dendritic cells (also referred to as myeloid dendritic cells) which typically secrete IL-12, IL-6, and TNF. DCs include plasmacytoid dendritic cells (pDC) which typically produce high amounts of interferon α. DCs may express certain markers including one or more of CD303, CD141, and CD1c.

“B Cell” or “B lymphocyte” is used in accordance with its plain and ordinary meaning in the art. B cells are lymphocytes, a type of white blood cell (leukocyte), that develops into a plasma cell (a “mature B cell”), which produces antibodies. B cells are antigen-presenting cells, which present processed antigen in that binds to receptors on the B cell surface (e.g. MHC-II molecules). For example, B cells may bind to an antigen by way of a B cell receptor. The antigen may be taken up into the B cell, processed, and the processed antigen may be presented on the surface of the B cell.

As used herein, the term “Treg” or “regulatory T cell” is used in accordance with its plain and ordinary meaning and refers to a subpopulation of T-cells that are capable of suppressing activation of the immune system. Treg cells typically express CD4, CD25, and Foxp3. Treg cells typically express low amounts of or no CD127. Treg cells suppress pro-inflammatory cytokine production and proliferation of T effector cells. In embodiments, populations of Treg cells may co-express IFN-g or IL-17. In embodiments, Treg cells proliferate when stimulated with TCR. According to antigen specificity, Treg cells can be classified into polyclonal Treg cells and antigen-specific Treg cells.

The term “antigen-specific regulatory T cell” or “aTreg” refer to a Treg cell that is immunosuppressive in response to an antigen or antigens, or maintains a state of non-responsiveness of the immune system in response to said antigen or antigens. Immunosuppressiveness or non-responsiveness of the immune system is one or more of decreased proliferation of effector T cells (e.g. cytotoxic T cells, CTLs, T-killer cells, killer T cells, etc.), increased production of immunosuppressive cytokines, decreased production of proinflammatory cytokines, or decreased cell lysis. In embodiments, immunosuppressiveness or non-responsiveness of the immune system is decreased production or activation of a transcription factor (e.g. NF-κB, C/EBP-β, etc.) that results in production or activation of a proinflammatory cytokine. In embodiments, the proinflammatory cytokine is interferon-gamma (IFN-gamma), tumor necrosis factor alpha (TNF-alpha), interleukin-4 (IL-4), or interleukin-6 (IL-6). In embodiments, the immunosuppressive cytokine is IL-10 and/or tumor growth factor-β (TGF-β). Detection of cytokine production can be detected using conventional techniques for detecting protein (e.g., ELISA, Western blotting, flow cytometry, immunofluorescence, immunohistochemistry, etc.). Methods for measuring cell lysis are well known in the art and include annexin V staining, flow cytometry, electron microscopy, and Western blotting. Other methods for measuring cell lysis are described in detail in Martinez, M. M. et al. Detection of apoptosis: A review of conventional and novel techniques, Anal. Methods, 2010, 2, 996-1004: DOI: 10.1039/c0ay00247j.; which is incorporated by reference herein in its entirety and for all purposes.

In embodiments, an aTreg is immunosuppressive or non-responsive to an antigen derived from an allogenic transplant/transfusion. An antigen-specific Treg may be generated by contacting a Treg from a tissue transplant donor with antigen presenting cells from the recipient of the transplant. Thus, the resultant aTreg (e.g. recipient antigen-specific Treg) is immunosuppressive in response (e.g. maintains tolerance) to an antigen or antigens from the recipient of the transplant.

As used herein, “recipient antigen presenting cells” or “recipient APCs” refer to immune cells taken from the recipient of an allogenic transplant/transfusion that are capable of processing and presenting antigen from the recipient. In embodiments, the recipient APCs present processed recipient antigens for recognition by certain lymphocytes, for example CD8+ T cells. Presentation of said recipient antigens may result in proliferating of CD8+ T cells. The recipient antigen presenting cells may include dendritic cells, B cells, and/or macrophages. Recipient antigen presenting cells may display recipient derived antigen on their surfaces, typically by forming complexes with the antigen through the major histocompatibility complex (MHC). Presentation of recipient derived antigens to T cells, for example CD4+ T cells, by recipient APCs may stimulate the T cells, thereby activating the immune response. In embodiments, the recipient antigen presenting cells are derived from transplanted tissue from the recipient of an allogeneic transplant/transfusion. In embodiments, contacting regulatory T cells (Treg cells) from the transplant donor with recipient antigen presenting cells forms a recipient antigen-specific regulatory T cell.

As used herein, “recipient antigen-specific regulatory T cell”, “recipient antigen-specific Treg” or “recipient aTreg” refers to a regulatory T cell (Treg cell) that has immunosuppressive properties or maintains a state of non-responsiveness of the immune system towards one or more antigens derived from the recipient of an allogeneic transplant/transfusion. As described herein, immunosuppressiveness or non-responsiveness of the immune system is one or more of decreased proliferation of effector T cells (e.g. cytotoxic T cells, CTLs, T-killer cells, killer T cells, etc.), increased production of immunosuppressive cytokines, decreased production of proinflammatory cytokines, or decreased cell lysis. In embodiments, immunosuppressiveness or non-responsiveness of the immune system is one or more of decreased production or activation of a transcription factor (e.g. NF-κB, C/EBP-β, etc.) that results in production or activation of a proinflammatory cytokine.

Recipient antigen-specific Treg cells are formed by contacting Treg cells with recipient antigen presenting cells or activated recipient antigen presenting cells. In embodiments, the Treg cells are contacted with recipient antigen presenting cells or activated recipient antigen presenting cells in the presence of a CD28 inhibitor. In embodiments, recipient antigen-specific Treg cells are formed by contacting Treg cells with recipient antigen presenting cells in the presence of a CD28 inhibitor. In embodiments, recipient antigen-specific Treg cells are formed by contacting Treg cells with activated recipient antigen presenting cells in the presence of a CD28 inhibitor. In embodiments, recipient antigen-specific regulatory T cell show immune tolerance (e.g. have immunosuppressive properties, maintains a state of non-responsiveness of the immune system) towards one or more recipient derived antigens.

As used herein, “activated recipient antigen presenting cell” refers to an antigen presenting cell taken from the recipient of an allogenic transplant/transfusion that is contacted with one or more recipient antigens and is stimulated by contacting with one or more proinflammatory stimuli (e.g. cytokines, liposaccharides, etc.). In embodiments, the proinflammatory stimuli includes IFN-γ, TNF, IL-1, a TLR9 agonist (CpG-containing nucleic acid. Thus, in embodiments, an activated recipient antigen presenting cell presents one or more recipient antigens and may promote proliferation of CD+ T cells. In embodiments, the recipient antigen presenting cells include dendritic cells, B cells, and/or macrophages. In embodiments, the recipient antigen presenting cells become activated upon contact with recipient antigen, thereby becoming activated recipient antigen presenting cells. In embodiments, contacting Treg cells with activated recipient antigen presenting cells forms recipient antigen-specific regulatory T cells.

As used herein, “different recipient antigen presenting cells” or “different recipient APCs” refer to a plurality cells where two or more of distinct types of antigen presenting immune cells are included in the plurality of cells. For example, the different recipient antigen presenting cells may include a combination of two or more types of antigen presenting cells. The two or more types of antigen presenting cells may include macrophages, B cells or dendritic cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents and methods well-known in the art (e.g. flow cytometry, etc.).

As used herein, the term “PBMC” or “peripheral blood mononuclear cell” is used in accordance with its plain and ordinary meaning and refers to a mixture of immune cells, including lymphocytes, monocytes, natural killer cells (NK cells) and dendritic cells. PBMCs may be characterized by their round nuclei, and may be separated from red blood cells and granulocytes in whole blood. Typically, lymphocytes are in the range of 70-90%, monocytes from 10 to 20%, while dendritic cells account for 1-2%. Typically, frequencies of cell types within the lymphocyte population include 70-85% CD3+ T cells, 5-10% B cells, and 5-20% NK cells.

The term “anergic” refers to a state of immune unresponsiveness. For example, an anergic T cell is a T cell that displays immune tolerance to an antigen. An anergic recipient antigen-specific T cell may have lower levels of proliferation or effector functions when encountered or contacted with an antigen from a tissue transplant recipient. In embodiments, the anergic T cell may express higher levels of immune suppressive cytokines (e.g. IL-10, transforming growth factor-β, etc.) compared to a T cell that is not anergic (e.g. a T cell that is immune responsive to an antigen from a recipient of a tissue transplant/transfusion).

The term “expand” as used herein refers to increasing or proliferating the number of cells in a cell culture. The culture medium may include growth factors, serum, cytokines or and other additives to help cells grow and/or differentiate.

Thus, “expanding regulatory T cells” or “expanding Treg cells” refers to the process of proliferating Treg cells in a cell culture, thereby forming a population of “expanded regulatory T cells” or “expanded Treg cells”. Treg cells may be expanded in the presence of one or more agents that assist in the proliferation or survival of the cells. The agent may be a small molecule, antibody, cell, or cytokine. For example the agent may be one or more of CD3, CD28, interleukin-2 (IL-2), β-mecaptothanol, penicillin, streptomycin, and rapamycin. In embodiments, the Treg cells may be expanded in the presence of one or more of antigen presenting cells (APC), rapamycin, interleukin 2 (IL-2), interleukin 15 (IL-15), anti-CD3 antibody, CD28 inhibitor compound (e.g. belatacept). In embodiments, expanding regulatory T cells comprises includes culturing Treg cells in the presence of CD3 and CD28. In embodiments, the Treg cells are from the donor of a tissue transplant/transfusion. Methods for expanding Treg cells are described in more detail in MacDonald, K. N. et al. Methods to manufacture regulatory T cells for cell therapy; British Society for Immunology, Clinical and Experimental Immunology, 2019, 197: 52-63; doi:10.1111/cei.13297. and; Putnam, A. L. et al. Expansion of Human Regulatory T-Cells From Patients With Type 1, Diabetes. 2009 March; 58(3): 652-662; doi: 10.2337/db08-1168; which are incorporated by reference herein in their entirety and for all purposes.

As used herein, the term “allogeneic transplant” or “allogeneic transfusion” refers to the transfer of biological material (e.g. tissue or blood) to a recipient from a genetically non-identical donor of the same species. The term “transplant” may be referred to as an allograft, allogeneic transplant, or homograft. For example, the allogeneic transplant may be a tissue transplant or organ transplant. Thus, an allogeneic transplant may include transfer of tissue, a group of cells or an organ to a recipient that is genetically non-identical to the donor. In embodiments, the transplant is a bone marrow transplant. In embodiment, the tissue transplant includes stem cells. In embodiment, the tissue transplant includes hematopoietic stem cells. In embodiments, the tissue transplant includes islet cells.

As used herein, “recipient antigen” refers to an antigen derived from the recipient of tissue transplant/transfusion. In embodiments, the recipient antigen is a tissue antigen. A tissue antigen (also referred to herein as a “recipient tissue antigen”) is an antigen derived from the allogeneic transplant of allogeneic transfusion that is produced by the recipient. In embodiments, the recipient antigen is an antigen taken from the transplanted tissue or cell. For example, the recipient antigen may be taken from the bone marrow transplant. For example, the recipient antigen may be taken from a tissue transplant including hematopoietic stem cells.

As used herein, “tissue transplant” refers to a group of cells, organs or tissue that is transferred from a donor to the recipient. In embodiments, the tissue transplant is an allogenic transplant. In embodiments, the tissue transplant is bone marrow. In embodiments, the tissue transplant includes stem cells. Organs include the heart, kidneys, liver, lungs, pancreas, intestine, thymus and uterus. Tissues include bones, tendons, corneae, skin, heart valves, nerves and veins.

As used herein, “transplanted bone marrow” refers to allogeneic stem cells that are transferred from a donor to the recipient. For example, recipient antigen presenting cells may be derived from transplanted bone marrow from the recipient.

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

As used herein, the terms “isolated”, “isolating”, “purified” refer to cells or molecules that have been separated from their natural milieu or from components of the environment in which they are produced. Thus, “isolated” or “isolating” do not necessarily refer to the degree of purity of a cell or molecule of the present invention. For example, a naturally occurring cell or molecule (e.g., a Treg cell, an APC, protein etc.) present in a living animal, including humans, is not isolated. However, the same cell, or molecule, separated from some or all of the coexisting materials in the animal, is considered isolated. As a further example, cells that are present in a sample of blood obtained from a person would be considered isolated. It should be appreciated that cells obtained from such a sample using further purification steps would also be referred to as isolated, in keeping with the notion that isolated does not refer to the degree of purity of the cells.

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

As defined herein, the term “inhibition”, “inhibit”, “inhibiting” and the like in reference to an inhibitor (e.g. antagonist) interaction means negatively affecting (e.g. decreasing) the level of activity or function of a cellular response relative to the level of activity or function of the cellular response in the absence of the inhibitor. The term “inhibitor” may be interchangeable with the term “blocker.” In embodiments, inhibition refers to reduction of a disease or symptoms of immune disease, such as GvHD. Thus, inhibition may include, at least in part, partially or totally blocking stimulation, decreasing, preventing, or delaying activation, or inactivating, desensitizing, or down-regulating signal transduction or enzymatic activity or the amount of a cellular response.

The term “CD28 inhibitor compound” refers to a molecule that modulates or downregulates CD28 protein activity, either directly or indirectly. CD28 is a protein expressed on T cells that provides co-stimulatory signals for T cell activation and survival. Further, CD28 stimulation of T cells assists in providing signals for the production of various pro-inflammatory cytokines, for example, interleukin-6 (IL-6). Thus, a CD28 inhibitor compound inhibits or downregulates CD28 signaling pathways, thereby decreasing T cell activation and/or proliferation. Thus, in embodiments, a CD28 inhibitor compound decreases or inhibits T cell effector properties (e.g. cell killing, expression of immune activating cytokines (.e.g. IL-2, IL-4, and IFN-γ, etc.)). In embodiments, the CD28 inhibitor is belatacept. In embodiments, the CD28 inhibitor is abatacept. CD28 inhibitors include CTLA4-Ig, abatacept, belatacept, FR104 and lulizumab.

The term “CD80/CD86 antagonist” refers to molecules that either inactivate or downregulate downstream signaling events of CD80/CD86 on the surfaces of APCs, thereby repressing costimlatory signaling between APCs and CD28 on T cells. CD80 is a receptor protein for CD28, and is found on the surface of immune cells, including antigen-presenting cells (e.g. dendritic cells). CD80 is involved in signaling that results in T and B-cell activation, and particularly in dendritic cell licensing and cytotoxic T-cell activation. Similarly, CD86 is expressed on antigen-presenting cells and is required for T-cell activation. CD80/CD86 inhibitors include RhuDex, CTLA-4—Ig, abatacept, and belatacept.

“Belatacept” is a fusion protein comprised of the Fc fragment of a human IgG1 immunoglobulin linked to the extracellular domain of CTLA-4. Belatacept selectively inhibits the process of T-cell activation and is a therapeutic for graft and transplant patients.

As used herein, the term “modulator” refers to a composition that increases or decreases the level of a target molecule or the function of a target molecule. The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

As defined herein, the term “activation”, “activate”, “activating” and the like in reference to an activator (e.g. agonist) interaction means positively affecting (e.g. increasing) the activity or function of a cellular response relative to the activity or function of the response in the absence of the activator. Thus, activation may include, at least in part, partially or totally increasing stimulation, increasing or enabling activation, or activating, sensitizing, or up-regulating signal transduction or enzymatic activity or the amount of a cellular response decreased in a disease. Activation may include, at least in part, partially or totally increasing stimulation, increasing or enabling activation, or activating, sensitizing, or up-regulating signal transduction or enzymatic activity or the amount of a cellular response.

In regard to immune cells and subsets of immune cells, the term “activate”, activated”, “activating” and the like mean the response of immune cells to a molecule, where the response is cell division and secretion of cytokines or proteins that regulate or assist the immune response. An activated immune cell may modulate the immune response by downregulating or activating the immune response. For example, a recipient-derived antigen presenting cell (eg. DCs or B cells) may be activated by an antigen. For example, Treg cells may be activated when triggered by an antigen, thereby mediating suppression of the immune response.

The term “derived from,” when referring to cells or a biological sample, indicates that the cell or sample was obtained from the stated source at some point in time. For example, a cell derived from an individual can represent a primary cell obtained directly from the individual (i.e., unmodified), or can be modified, e.g., by introduction of a recombinant vector, by culturing under particular conditions, or immortalization. In some cases, a cell derived from a given source will undergo cell division and/or differentiation such that the original cell is no longer exists, but the continuing cells will be understood to derive from the same source.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

As used herein, the term “patient” or “subject in need thereof” or “subject” refers to a living organism suffering from or prone to a disease (e.g. graft-versus-host disease, cancer, etc.) or condition that can be treated by administration of a compound or pharmaceutical composition or by a method, as provided herein. Non-limiting examples include humans, other mammals, bovines, rats, mice, dogs, monkeys, goat, sheep, cows, deer, and other non-mammalian animals. In embodiments, a patient is human. In embodiments, a subject is human. The terms individual, subject, and patient by themselves do not denote a particular age, sex, race, and the like. Thus, individuals of any age, whether male or female, are intended to be covered by the present disclosure. Likewise, the methods of the present invention can be applied to any race, including, for example, Caucasian (white), African-American (black), Native American, Native Hawaiian, Hispanic, Latino, Asian, and European.

In embodiments, the subject in need thereof is the recipient of a tissue transplant/transfusion. In embodiments, the subject in need thereof is the patient in which graft vs host disease is being prevented. In embodiments, the subject in need thereof is the patient in which graft vs host disease is being treated.

As used herein, the term “recipient” refers to the subject that is receiving the tissue transplant or transfusion from a donor, wherein the donor is not genetically identical to the recipient. For example, the recipient may be receiving an organ, tissue, or group of cells from the donor. For example, the recipient may be receiving a bone marrow transplant from the donor. In embodiments, the recipient suffers from GvHD.

As used herein, the term “donor” refers to a subject who provides an organ, tissue, or group of cells for transplantation to a recipient, wherein the donor is not genetically identical to the recipient. For example, a donor may provide a donated tissue (bone) to a subject in need thereof. In embodiments, a donor is healthy, e.g. does not suffer from GvHD.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. For example, administration may be infusion of recipient antigen-specific Treg cells provided herein including embodiments thereof into a subject in need thereof. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols. The preparations may also be combined with inhaled mucolytics (e.g., rhDNase, as known in the art) or with inhaled bronchodilators (short or long acting beta agonists, short or long acting anticholinergics), inhaled corticosteroids, or inhaled antibiotics to improve the efficacy of these drugs by providing additive or synergistic effects. The compositions of the present invention can be delivered transdermally, by a topical route, formulated as applicator sticks, solutions, suspensions, emulsions, gels, creams, ointments, nanoparticles, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

As used herein, the term “associated” or “associated with” in the context of a substance or substance activity or function associated with a disease (such as GvHD) means that the disease is caused by (in whole or in part), or a symptom of the disease is caused by (in whole or in part) the substance or substance activity or function. As used herein, what is described as being associated with a disease, if a causative agent, could be a target for treatment of the disease. For example, GvHD may be treated with a composition (e.g. compound, composition, nanoparticle, or conjugate, all as described herein) effective for inhibiting or preventing T cell effector activity from the donated tissue transplant.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

As used herein, the term “pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

As used herein, the term “preparation” is intended to include the formulation of the active compound or cells with encapsulating or solution material as a carrier.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In embodiments, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of a disease or disease symptoms in a patient. As indicated above, the prevention may be complete (no detectable symptoms) or partial, such that fewer symptoms are observed than would likely occur absent treatment.

As used herein, the term “graft-versus-host disease” or “GvHD” refers to a medical complication following the receipt of transplanted tissue from a genetically different person. GvHD is commonly associated with stem cell transplants such as those that occur with bone marrow transplants. GvHD also applies to other forms of transplanted tissues such as solid organ transplants. In GvHD, white blood cells of the donor's immune system which remain within the donated tissue (the graft) recognize the recipient (the host) as foreign (non-self). The white blood cells present within the transplanted tissue then attack the recipient's body's cells, which leads to GvHD. This should not be confused with a transplant rejection, which occurs when the immune system of the transplant recipient rejects the transplanted tissue; GvHD occurs when the donor's immune system's white blood cells reject the recipient. The underlying principle (alloimmunity) is the same, but the details and course may differ. GvHD can also occur after a blood transfusion if the blood products used have not been irradiated or treated with an approved pathogen reduction system. In embodiments, the graft-versus-host-disease is chronic graft-versus-host-disease.

“Immune diseases” include graft-versus-host disease, acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, Henoch-Schonlein purpura, post-streptococcalnephritis, erythema nodosum, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, pemphigus vulgaris, Wegener's granulomatosis, membranous nephropathy, amyotrophic lateral sclerosis, tabes dorsalis, giant cell arteritis/polymyalgia, perniciousanemia, rapidly progressive glomerulonephritis, psoriasis, and fibrosing alveolitis. Symptoms of immune diseases such as fatigue, joint pain and swelling, skin problems, abdominal pain or digestive issues, recurring fever, and swollen glands would be known or may be determined by a person of ordinary skill in the art.

The term “cancer” refers to all types of cancer, neoplasm or malignant tumors found in mammals (e.g. humans), including leukemias, lymphomas, carcinomas and sarcomas. Exemplary cancers that may be treated with compounds, nucleic acids, and pharmaceutical compositions described herein include leukemia (e.g., acute myeloid leukemia (“AML”) or chronic myeloid leukemia (“CML”)) brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's disease, and Non-Hodgkin's lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. In embodiments, the cancer is glioma. In embodiments, the glioma is astrocytoma (e.g., astrocytoma, anaplastic astrocytoma, glioblastoma). In embodiments, the glioma is ependymoma (e.g., anaplastic ependymoma, myxopapillary ependymoma, subependymoma). In embodiments, the glioma is oligodendroglioma (e.g., oligodendroglioma, anaplastic oligodendroglioma, anaplastic oligoastrocytoma). In embodiments, the glioma is a brain stem glioma. In embodiments, the glioma is a mixed glioma. In embodiments, the glioma is an optic pathway glioma.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute myeloid leukemia, chronic myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “red blood cell disease” refers to a disease affecting a red blood cell. Non-limiting examples of red blood cell diseases include anemia, sickle cell disease, acute lymphoblastic leukemia, hemolytic anemia, aplastic anemia, thalassemia, polycythemia, myelodysplastic syndrome, polycythemia vera, iron-deficiency anemia, autoimmune hemolytic anemia, sphercytosis, hereditary spherocytosis, megaloblastic anemia, glucose-6-phosphate dehydrogenase deficiency, normocytic anemia, paroxysmal nocturnal hemoglobinuria, hypochromic anemia, macrocytic anemia, pyruvate kinase deficiency, hereditary stomatocytosis, microcytosis, microcytic anemia, macrocytosis and hereditary elliptocytosis.

In some instances, “disease” or “condition” refer to “hematological disease.” A hematological disease refers to a disease affecting a hematologic cell. In some instances, the hematological disease is a non-cancerous (i.e. non-malignant) hematological disease. Non-cancerous hematological diseases as provided herein include any disease, disorder or condition related to hematologic cells that is not cancer. Examples of non-cancerous hematological diseases, disorders, or conditions include, but are not limited to hemoglobinopathies including sickle-cell disease, thalassemia, methemoglobinemia; anemias including iron deficiency anemia, folate deficiency, hemolytic anemias, megaloblastic anemia, vitamin B12 deficiency, pernicious anemia, immune mediated hemolytic anemia, drug-induced immune mediated hemolytic anemia (e.g. due to high dose of penicillin, methyldopa), hemoglobinopathies, paroxysmal nocturnal hemoglobinuria, and microangiopathic hemolytic anemia; disease characterized by decreased numbers of blood cells (e.g. erythrocytes, lymphocytes, myeloid cells) including myelodysplastic syndrome, myelofibrosis, neutropenia, agranulocytosis, Glanzmann's thrombasthenia, thrombocytopenia, idiopathic thrombocytopenic purpura, thrombotic thrombocytopenic purpura, and heparin-induced thrombocytopenia; myeloproliferative disorders including polycythemia vera, erythrocytosis, leukocytosis, and thrombocytosis; coagulopathies including thrombocytosis, recurrent thrombosis, disseminated intravascular coagulation, hemophilia, Von Willebrand disease, disseminated intravascular coagulation, protein S deficiency, and antiphospholipid syndrome.

As used herein, the term “effective amount” is an amount sufficient to accomplish a stated purpose (e.g. achieve the effect for which it is administered, treat a disease, reduce enzyme activity, increase enzyme activity, reduce protein function, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug or prodrug is an amount of a drug or prodrug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from binding assays or cell culture assays. Target concentrations will be those concentrations of active compound(s) that are capable of achieving the methods described herein, as measured using the methods described herein or known in the art.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

### Methods of Treatment

Provided herein, inter alia, are methods effective for treating and/or preventing graft vs host disease (GvHD). Methods provided herein including embodiments thereof are contemplated to be effective for inhibiting and/or preventing activation of immune cells present in a donated tissue transplant (e.g. bone marrow, hematopoietic stem cells, blood, etc.). Preventing and/or inhibiting activation and proliferation of donor inflammatory cells in a tissue transplant may prevent destruction of tissue in a transplant recipient. Thus, in an aspect is provided a method of treating or preventing graft-versus-host disease in a subject in need thereof, the method including administering to the subject a therapeutically effective amount of recipient antigen-specific regulatory T cells, thereby treating or preventing graft-versus-host disease in the subject; wherein the recipient antigen-specific regulatory T cells are derived from regulatory T cells from a donor of a tissue transplant to the subject; and wherein the tissue transplant is a cause of graft-versus-host disease in the subject.

The recipient antigen-specific regulatory T cells are effective in treating or preventing graft-versus host disease compared to non-specific (e.g. polyclonal) regulatory T cells. In embodiments, the graft-versus-host disease is chronic graft-versus-host disease. Treating or preventing graft-versus host disease refers to reduction or disappearance of symptoms of graft-versus host disease. Symptoms of graft versus-host disease may include one or more of dry mouth, sensitivity to hot, cold, spicy and acidic foods, sensitivity to mint, sensitivity to carbonated drinks, mouth ulcers, mouth ulcers that extend down the throat, difficulty eating, gum disease, tooth decay, rash, dry, tight or itchy skin, thickening and tightening of the skin, restriction of joint movement, change in skin color, intolerance to temperature changes due to damaged sweat glands, changes in nail texture, hard brittle nails, loss of body hair, loss of appetite, unexplained weight loss, nausea, vomiting, diarrhea, stomach pain, difficulty breathing, wheezing, persistent chronic cough, abdominal swelling, jaundice, abnormal liver function, muscle weakness, joint stiffness, vaginal dryness, itching and pain, genital ulcerations and scarring, narrowing or the urethra. Symptoms of chronic graft-versus-host disease are well-known and are described in further detail in Lee, S. J. et al., Chronic graft-versus-host disease; Biol. Blood Marrow Transplant. Volume 9, Issue 4, April 2003, Pages 215-233: https://doi.org/10.1053/bbmt.2003.50026; which is incorporated by reference herein in its entirety and for all purposes. In embodiments, recipient antigen-specific regulatory T cells are at least 1.5 fold, 2 fold, 3 fold, 4 fold, 5 fold, 6 fold, 7 fold, 8 fold 9 fold or 10 fold more effective in treating or preventing GvHD compared to non-specific regulatory T cells. In embodiments, recipient antigen-specific regulatory T cells are at least 10 fold more effective in treating or preventing GvHD compared to non-specific regulatory T cells.

GvHD, particularly chronic GvHD, may not manifest in a subject until at least, 3 months, 4 months, 5 months, 6 months after the subject receives the tissue transplant. At the time the subject has GvHD, the donor may not be available to donate Treg cells. Thus, in embodiments, regulatory T cells from the donor are obtained within a time period wherein the donor is available to donate said regulatory T cells. In embodiments, obtaining Treg cells from said donor includes taking blood from the donor, isolating Treg cells from the blood and storing the Treg cells. Obtaining donor regulatory T cells may include isolating regulatory T cells from the blood of said donor. In embodiments, obtaining donor Treg cells includes expanding the donor Treg cells. Methods for obtaining donor Treg cells are described in greater detail in Putnam A L, Brusko T M, Lee M R, Liu W, Szot G L, Ghosh T, et al. Expansion of human regulatory T-cells from patients with type 1 diabetes. Diabetes. 2009; 58(3):652-62; which is incorporated by reference herein in its entirety and for all purposes.

Thus, in embodiments, the regulatory T cells are obtained from the donor during the procedure to remove the tissue transplant from the donor. In embodiments, the regulatory T cells are obtained from the donor within 24 hrs of the procedure to remove the tissue transplant from the donor. In embodiments, the regulatory T cells are obtained from the donor within one week the tissue transplant was obtained from the donor. In embodiments, the regulatory T cells are obtained from the donor within two weeks the tissue transplant was obtained from the donor. In embodiments, the regulatory T cells are obtained from the donor within three weeks the tissue transplant was obtained from the donor. In embodiments, the regulatory T cells are obtained from the donor within one month the tissue transplant was obtained from the donor. In embodiments, the regulatory T cells are obtained from the donor within two months the tissue transplant was obtained from the donor. In embodiments, the regulatory T cells are obtained from the donor within three months the tissue transplant was obtained from the donor. In embodiments, the regulatory T cells are obtained from the donor within four months the tissue transplant was obtained from the donor. In embodiments, the regulatory T cells are obtained from the donor within five months the tissue transplant was obtained from the donor. In embodiments, the regulatory T cells are obtained from the donor within six months the tissue transplant was obtained from the donor. In embodiments, the regulatory T cells are obtained from the donor within eight months the tissue transplant was obtained from the donor. In embodiments, the regulatory T cells are obtained from the donor within ten months the tissue transplant was obtained from the donor. In embodiments, the regulatory T cells are obtained from the donor within one year the tissue transplant was obtained from the donor. In embodiments, the regulatory T cells are isolated from the donor's blood.

In embodiments, the tissue transplant is bone marrow. In embodiments, the tissue transplant includes hematopoietic stem cells.

In embodiments, the subject (e.g. patient, recipient) has or previously had cancer. In embodiments, the cancer is leukemia, lymphoma, sarcoma, myeloma, or glioma. In embodiments, the cancer is leukemia. In embodiments, the cancer is lymphoma. In embodiments, the cancer is sarcoma. In embodiments, the cancer is myeloma. In embodiments, the cancer is glioma. In embodiments, the subject has or previously had a red blood cell disease. In embodiments, the red blood cell disease is anemia, sickle cell disease, acute lymphoblastic leukemia, hemolytic anemia, aplastic anemia, thalassemia. In embodiments, the red blood cell disease is anemia. In embodiments, the red blood cell disease is sickle cell disease. In embodiments, the red blood cell disease is acute lymphoblastic leukemia. In embodiments, the red blood cell disease is hemolytic anemia. In embodiments, the red blood cell disease is aplastic anemia. In embodiments, the red blood cell disease is thalassemia.

For the methods provided herein including embodiments thereof, the recipient antigen-specific regulatory T cells are formed by a method including: (a) expanding the regulatory T cells in vitro, thereby forming a plurality of regulatory T cells; and (b) contacting the plurality of regulatory T cells with a plurality of recipient antigen presenting cells and a CD28 inhibitor compound in vitro, thereby forming the recipient antigen-specific regulatory T cells. In embodiments, step (b) further includes contacting the plurality of regulatory T cells with a second plurality of recipient antigen presenting cells. In embodiments, the plurality of recipient antigen presenting cells are taken from the subject's blood. In embodiments, the second plurality of recipient antigen presenting cells are taken from the subject's blood. In embodiments, the plurality of recipient antigen presenting cells are taken from the subject's tissue transplant. In embodiments, the second plurality of recipient antigen presenting cells are taken from the subject's tissue transplant.

For the methods provided herein, in embodiments, during the contacting of the plurality of regulatory T cells with the plurality of recipient antigen presenting cells, the number of Treg cells are greater than the number of recipient APCs in culture. In embodiments, the ratio of the plurality of Treg cells to the plurality of recipient APCs is 2:1. In embodiments, the ratio of the plurality of Treg cells to the plurality of recipient APCs is 3:1 (Treg cell:recipient APC). In embodiments, the ratio of the plurality of Treg cells to the plurality of recipient APCs is 4:1 (Treg cell:recipient APC). In embodiments, the ratio of the plurality of Treg cells to the plurality of recipient APCs is 5:1 (Treg cell:recipient APC). In embodiments, the ratio of the plurality of Treg cells to the plurality of recipient APCs is 6:1 (Treg cell:recipient APC). In embodiments, the ratio of the plurality of Treg cells to the plurality of recipient APCs is 7:1 (Treg cell:recipient APC). In embodiments, the ratio of the plurality of Treg cells to the plurality of recipient APCs is 8:1 (Treg cell:recipient APC). In embodiments, the ratio of the plurality of Treg cells to the plurality of recipient APCs is 9:1 (Treg cell:recipient APC). In embodiments, the ratio of the plurality of Treg cells to the plurality of recipient APCs is 10:1 (Treg cell:recipient APC). In embodiments, the ratio of the plurality of Treg cells to the plurality of recipient APCs is 11:1 (Treg cell:recipient APC). In embodiments, the ratio of the plurality of Treg cells to the plurality of recipient APCs is 12:1. In embodiments, the ratio of the plurality of Treg cells to the plurality of recipient APCs is 13:1 (Treg cell:recipient APC). In embodiments, the ratio of the plurality of Treg cells to the plurality of recipient APCs is 14:1 (Treg cell:recipient APC). In embodiments, the ratio of the plurality of Treg cells to the plurality of recipient APCs is 15:1 (Treg cell:recipient APC). In embodiments, the ratio of the plurality of Treg cells to the plurality of recipient APCs is 16:1 (Treg cell:recipient APC). In embodiments, the ratio of the plurality of Treg cells to the plurality of recipient APCs is 17:1 (Treg cell:recipient APC). In embodiments, the ratio of the plurality of Treg cells to the plurality of recipient APCs is 18:1 (Treg cell:recipient APC). In embodiments, the ratio of the plurality of Treg cells to the plurality of recipient APCs is 19:1 (Treg cell:recipient APC). In embodiments, the ratio of the plurality of Treg cells to the plurality of recipient APCs is 20:1 (Treg cell:recipient APC).

In embodiments, the contacting the plurality of regulatory T cells with a second plurality of recipient antigen presenting cells includes adding the second plurality of recipient antigen presenting cells to the cell culture. In embodiments, after adding the second plurality of recipient APC to the cell culture, the ratio of the Treg cell to recipient APC is 1:1 (Treg cell:recipient APC). In embodiments, after adding the second plurality of recipient APC to the cell culture, the ratio of the Treg cell to recipient APC is 2:1 (Treg cell:recipient APC). In embodiments, after adding the second plurality of recipient APC to the cell culture, the ratio of the Treg cell to recipient APC is 3:1 (Treg cell:recipient APC). In embodiments, after adding the second plurality of recipient APC to the cell culture, the ratio of the Treg cell to recipient APC is 4:1 (Treg cell:recipient APC). In embodiments, after adding the second plurality of recipient APC to the cell culture, the ratio of the Treg cell to recipient APC is 5:1. In embodiments, after adding the second plurality of recipient APC to the cell culture, the ratio of the Treg cell to recipient APC is 6:1 (Treg cell:recipient APC). In embodiments, after adding the second plurality of recipient APC to the cell culture, the ratio of the Treg cell to recipient APC is 7:1 (Treg cell:recipient APC). In embodiments, after adding the second plurality of recipient APC to the cell culture, the ratio of the Treg cell to recipient APC is 8:1 (Treg cell:recipient APC). In embodiments, after adding the second plurality of recipient APC to the cell culture, the ratio of the Treg cell to recipient APC is 9:1 (Treg cell:recipient APC). In embodiments, after adding the second plurality of recipient APC to the cell culture, the ratio of the Treg cell to recipient APC is 10:1 (Treg cell:recipient APC).

In embodiments, the plurality of regulatory T cells are contacted with the plurality of recipient antigen presenting cells in the presence of a CD28 inhibitor compound and IL-2. In embodiments, the concentration of IL-2 is from 0.5 International units per milliliter (IU/mL) to 20 IU/mL. In embodiments, the concentration of IL-2 is from 2 IU/mL to 20 IU/mL. In embodiments, the concentration of IL-2 is from 4 IU/mL to 20 IU/mL. In embodiments, the concentration of IL-2 is from 6 IU/mL to 20 IU/mL. In embodiments, the concentration of IL-2 is from 8 IU/mL to 20 IU/mL. In embodiments, the concentration of IL-2 is from 10 IU/mL to 20 IU/mL. In embodiments, the concentration of IL-2 is from 12 IU/mL to 20 IU/mL. In embodiments, the concentration of IL-2 is from 14 IU/mL to 20 IU/mL. In embodiments, the concentration of IL-2 is from 16 IU/mL to 20 IU/mL. In embodiments, the concentration of IL-2 is from 18 IU/mL to 20 IU/mL.

In embodiments, the concentration of IL-2 is from 0.5 IU/mL to 18 IU/mL. In embodiments, the concentration of IL-2 is from 0.5 IU/mL to 16 IU/mL. In embodiments, the concentration of IL-2 is from 0.5 IU/mL to 14 IU/mL. In embodiments, the concentration of IL-2 is from 0.5 IU/mL to 12 IU/mL. In embodiments, the concentration of IL-2 is from 0.5 IU/mL to 10 IU/mL. In embodiments, the concentration of IL-2 is from 0.5 IU/mL to 8 IU/mL. In embodiments, the concentration of IL-2 is from 0.5 IU/mL to 6 IU/mL. In embodiments, the concentration of IL-2 is from 0.5 IU/mL to 4 IU/mL. In embodiments, the concentration of IL-2 is from 0.5 IU/mL to 2 IU/mL. In embodiments, the concentration of IL-2 is 0.5 IU/mL, 2 IU/mL, 4 IU/mL, 6 IU/mL, 8 IU/mL, 10 IU/mL, 12 IU/mL, 14 IU/mL, 16 IU/mL, 18 IU/mL, or 20 IU/mL. In embodiments, the concentration of IL-2 is 10 IU/mL.

For the methods provided herein, in embodiments, the plurality of regulatory T cells are contacted with the plurality of recipient antigen presenting cells for 0.5 days to 5 days. In embodiments, the plurality of regulatory T cells are contacted with the plurality of recipient antigen presenting cells for 1 days to 5 days. In embodiments, the plurality of regulatory T cells are contacted with the plurality of recipient antigen presenting cells for 1.5 days to 5 days. In embodiments, the plurality of regulatory T cells are contacted with the plurality of recipient antigen presenting cells for 2 days to 5 days. In embodiments, the plurality of regulatory T cells are contacted with the plurality of recipient antigen presenting cells for 2.5 days to 5 days. In embodiments, the plurality of regulatory T cells are contacted with the plurality of recipient antigen presenting cells for 3 days to 5 days. In embodiments, the plurality of regulatory T cells are contacted with the plurality of recipient antigen presenting cells for 3.5 days to 5 days. In embodiments, the plurality of regulatory T cells are contacted with the plurality of recipient antigen presenting cells for 4 days to 5 days. In embodiments, the plurality of regulatory T cells are contacted with the plurality of recipient antigen presenting cells for 4.5 days to 5 days.

In embodiments, the plurality of regulatory T cells are contacted with the plurality of recipient antigen presenting cells for 0.5 days to 4.5 days. In embodiments, the plurality of regulatory T cells are contacted with the plurality of recipient antigen presenting cells for 0.5 days to 4 days. In embodiments, the plurality of regulatory T cells are contacted with the plurality of recipient antigen presenting cells for 0.5 days to 3.5 days. In embodiments, the plurality of regulatory T cells are contacted with the plurality of recipient antigen presenting cells for 0.5 days to 3 days. In embodiments, the plurality of regulatory T cells are contacted with the plurality of recipient antigen presenting cells for 0.5 days to 2.5 days. In embodiments, the plurality of regulatory T cells are contacted with the plurality of recipient antigen presenting cells for 0.5 days to 2 days. In embodiments, the plurality of regulatory T cells are contacted with the plurality of recipient antigen presenting cells for 0.5 days to 1.5 days. In embodiments, the plurality of regulatory T cells are contacted with the plurality of recipient antigen presenting cells for 0.5 days to 1 day. In embodiments, the plurality of regulatory T cells are contacted with the plurality of recipient antigen presenting cells for 0.5 days, 1 day, 1.5 days, 2 days, 2.5 days, 3 days, 3.5 days, 4 days, 4.5 days, or 5 days. In embodiments, the plurality of regulatory T cells are contacted with the plurality of recipient antigen presenting cells for 3 days.

For the methods provided herein, in embodiments, the plurality of regulatory T cells are contacted with the second plurality of recipient antigen presenting cells for 0.5 days to 5 days. In embodiments, the plurality of regulatory T cells are contacted with the second plurality of recipient antigen presenting cells for 1 days to 5 days. In embodiments, the plurality of regulatory T cells are contacted with the second plurality of recipient antigen presenting cells for 1.5 days to 5 days. In embodiments, the plurality of regulatory T cells are contacted with the second plurality of recipient antigen presenting cells for 2 days to 5 days. In embodiments, the plurality of regulatory T cells are contacted with the second plurality of recipient antigen presenting cells for 2.5 days to 5 days. In embodiments, the plurality of regulatory T cells are contacted with the second plurality of recipient antigen presenting cells for 3 days to 5 days. In embodiments, the plurality of regulatory T cells are contacted with the second plurality of recipient antigen presenting cells for 3.5 days to 5 days. In embodiments, the plurality of regulatory T cells are contacted with the second plurality of recipient antigen presenting cells for 4 days to 5 days. In embodiments, the plurality of regulatory T cells are contacted with the second plurality of recipient antigen presenting cells for 4.5 days to 5 days.

In embodiments, the plurality of regulatory T cells are contacted with the second plurality of recipient antigen presenting cells for 0.5 days to 4.5 days. In embodiments, the plurality of regulatory T cells are contacted with the second plurality of recipient antigen presenting cells for 0.5 days to 4 days. In embodiments, the plurality of regulatory T cells are contacted with the second plurality of recipient antigen presenting cells for 0.5 days to 3.5 days. In embodiments, the plurality of regulatory T cells are contacted with the second plurality of recipient antigen presenting cells for 0.5 days to 3 days. In embodiments, the plurality of regulatory T cells are contacted with the second plurality of recipient antigen presenting cells for 0.5 days to 2.5 days. In embodiments, the plurality of regulatory T cells are contacted with the second plurality of recipient antigen presenting cells for 0.5 days to 2 days. In embodiments, the plurality of regulatory T cells are contacted with the second plurality of recipient antigen presenting cells for 0.5 days to 1.5 days. In embodiments, the plurality of regulatory T cells are contacted with the second plurality of recipient antigen presenting cells for 0.5 days to 1 day. In embodiments, the plurality of regulatory T cells are contacted with the second plurality of recipient antigen presenting cells for 0.5 days, 1 day, 1.5 days, 2 days, 2.5 days, 3 days, 3.5 days, 4 days, 4.5 days, or 5 days. In embodiments, the plurality of regulatory T cells are contacted with the second plurality of recipient antigen presenting cells for 3 days.

For the methods provided herein, in embodiments, the Treg cells are expanded from 2 fold to more than 200 fold compared to the original number or concentration of Treg cells in the cell culture. In embodiments, the Treg cells are expanded from 8 fold to more than 200 fold compared to the original number or concentration of Treg cells in the cell culture. In embodiments, the Treg cells are expanded from 16 fold to more than 200 fold compared to the original number or concentration of Treg cells in the cell culture. In embodiments, the Treg cells are expanded from 32 fold to more than 200 fold compared to the original number or concentration of Treg cells in the cell culture. In embodiments, the Treg cells are expanded from 40 fold to more than 200 fold compared to the original number or concentration of Treg cells in the cell culture. In embodiments, the Treg cells are expanded from 48 fold to more than 200 fold compared to the original number or concentration of Treg cells in the cell culture. In embodiments, the Treg cells are expanded from 56 fold to more than 200 fold compared to the original number or concentration of Treg cells in the cell culture. In embodiments, the Treg cells are expanded from 64 fold to more than 200 fold compared to the original number or concentration of Treg cells in the cell culture. In embodiments, the Treg cells are expanded from 72 fold to more than 200 fold compared to the original number or concentration of Treg cells in the cell culture. In embodiments, the Treg cells are expanded from 88 fold to more than 200 fold compared to the original number or concentration of Treg cells in the cell culture. In embodiments, the Treg cells are expanded from 98 fold to more than 200 fold compared to the original number or concentration of Treg cells in the cell culture. In embodiments, the Treg cells are expanded from 104 fold to more than 200 fold compared to the original number or concentration of Treg cells in the cell culture. In embodiments, the Treg cells are expanded from 112 fold to more than 200 fold compared to the original number or concentration of Treg cells in the cell culture. In embodiments, the Treg cells are expanded from 120 fold to more than 200 fold compared to the original number or concentration of Treg cells in the cell culture. In embodiments, the Treg cells are expanded from 128 fold to more than 200 fold compared to the original number or concentration of Treg cells in the cell culture. In embodiments, the Treg cells are expanded from 136 fold to more than 200 fold compared to the original number or concentration of Treg cells in the cell culture. In embodiments, the Treg cells are expanded from 144 fold to more than 200 fold compared to the original number or concentration of Treg cells in the cell culture. In embodiments, the Treg cells are expanded from 152 fold to more than 200 fold compared to the original number or concentration of Treg cells in the cell culture. In embodiments, the Treg cells are expanded from 160 fold to more than 200 fold compared to the original number or concentration of Treg cells in the cell culture. In embodiments, the Treg cells are expanded from 168 fold to more than 200 fold compared to the original number or concentration of Treg cells in the cell culture. In embodiments, the Treg cells are expanded from 176 fold to more than 200 fold compared to the original number or concentration of Treg cells in the cell culture. In embodiments, the Treg cells are expanded from 184 fold to more than 200 fold compared to the original number or concentration of Treg cells in the cell culture. In embodiments, the Treg cells are expanded from 192 fold to more than 200 fold compared to the original number or concentration of Treg cells in the cell culture.

In embodiments, the Treg cells are expanded from 2 fold to 192 fold compared to the original number or concentration of Treg cells in the cell culture. In embodiments, the Treg cells are expanded from 2 fold to 184 fold compared to the original number or concentration of Treg cells in the cell culture. In embodiments, the Treg cells are expanded from 2 fold to 176 fold compared to the original number or concentration of Treg cells in the cell culture. In embodiments, the Treg cells are expanded from 2 fold to 168 fold compared to the original number or concentration of Treg cells in the cell culture. In embodiments, the Treg cells are expanded from 2 fold to 160 fold compared to the original number or concentration of Treg cells in the cell culture. In embodiments, the Treg cells are expanded from 2 fold to 152 fold compared to the original number or concentration of Treg cells in the cell culture. In embodiments, the Treg cells are expanded from 2 fold to 144 fold compared to the original number or concentration of Treg cells in the cell culture. In embodiments, the Treg cells are expanded from 2 fold to 136 fold compared to the original number or concentration of Treg cells in the cell culture. In embodiments, the Treg cells are expanded from 2 fold to 128 fold compared to the original number or concentration of Treg cells in the cell culture. In embodiments, the Treg cells are expanded from 2 fold to 120 fold compared to the original number or concentration of Treg cells in the cell culture. In embodiments, the Treg cells are expanded from 2 fold to 112 fold compared to the original number or concentration of Treg cells in the cell culture. In embodiments, the Treg cells are expanded from 2 fold to 104 fold compared to the original number or concentration of Treg cells in the cell culture. In embodiments, the Treg cells are expanded from 2 fold to 88 fold compared to the original number or concentration of Treg cells in the cell culture. In embodiments, the Treg cells are expanded from 2 fold to 80 fold compared to the original number or concentration of Treg cells in the cell culture. In embodiments, the Treg cells are expanded from 2 fold to 72 fold compared to the original number or concentration of Treg cells in the cell culture. In embodiments, the Treg cells are expanded from 2 fold to 64 fold compared to the original number or concentration of Treg cells in the cell culture. In embodiments, the Treg cells are expanded from 2 fold to 56 fold compared to the original number or concentration of Treg cells in the cell culture. In embodiments, the Treg cells are expanded from 2 fold to 48 fold compared to the original number or concentration of Treg cells in the cell culture. In embodiments, the Treg cells are expanded from 2 fold to 40 fold compared to the original number or concentration of Treg cells in the cell culture. In embodiments, the Treg cells are expanded from 2 fold to 32 fold compared to the original number or concentration of Treg cells in the cell culture. In embodiments, the Treg cells are expanded from 2 fold to 24 fold compared to the original number or concentration of Treg cells in the cell culture. In embodiments, the Treg cells are expanded from 2 fold to 16 fold compared to the original number or concentration of Treg cells in the cell culture. In embodiments, the Treg cells are expanded from 2 fold to 8 fold compared to the original number or concentration of Treg cells in the cell culture. In embodiments, the Treg cells are expanded from 2 fold, 8 fold, 16 fold, 24 fold, 32 fold, 40 fold, 48 fold, 56 fold, 64 fold, 72 fold, 80 fold, 88 fold, 96 fold, 104 fold, 112 fold, 120 fold, 128 fold, 136 fold, 144 fold, 152 fold, 160 fold, 168 fold, 176 fold, 184 fold, 192 fold, or 200 fold compared to the original number or concentration of Treg cells in the cell culture. In embodiments, the Treg cells are expanded 100 fold compared to the original number or concentration of Treg cells in the cell culture.

For the methods provided herein, in embodiments, the Treg cells are expanded from 100 fold to more than 3000 fold compared to the original number or concentration of Treg cells in the cell culture. In embodiments, the Treg cells are expanded from 200 fold to more than 3000 fold compared to the original number or concentration of Treg cells in the cell culture. In embodiments, the Treg cells are expanded from 300 fold to more than 3000 fold compared to the original number or concentration of Treg cells in the cell culture. In embodiments, the Treg cells are expanded from 400 fold to more than 3000 fold compared to the original number or concentration of Treg cells in the cell culture. In embodiments, the Treg cells are expanded from 500 fold to more than 3000 fold compared to the original number or concentration of Treg cells in the cell culture. In embodiments, the Treg cells are expanded from 600 fold to more than 3000 fold compared to the original number or concentration of Treg cells in the cell culture. In embodiments, the Treg cells are expanded from 700 fold to more than 3000 fold compared to the original number or concentration of Treg cells in the cell culture. In embodiments, the Treg cells are expanded from 800 fold to more than 3000 fold compared to the original number or concentration of Treg cells in the cell culture. In embodiments, the Treg cells are expanded from 900 fold to more than 3000 fold compared to the original number or concentration of Treg cells in the cell culture. In embodiments, the Treg cells are expanded from 1000 fold to more than 3000 fold compared to the original number or concentration of Treg cells in the cell culture. In embodiments, the Treg cells are expanded from 1100 fold to more than 3000 fold compared to the original number or concentration of Treg cells in the cell culture. In embodiments, the Treg cells are expanded from 1200 fold to more than 3000 fold compared to the original number or concentration of Treg cells in the cell culture. In embodiments, the Treg cells are expanded from 1300 fold to more than 3000 fold compared to the original number or concentration of Treg cells in the cell culture. In embodiments, the Treg cells are expanded from 1400 fold to more than 3000 fold compared to the original number or concentration of Treg cells in the cell culture. In embodiments, the Treg cells are expanded from 1500 fold to more than 3000 fold compared to the original number or concentration of Treg cells in the cell culture. In embodiments, the Treg cells are expanded from 1600 fold to more than 3000 fold compared to the original number or concentration of Treg cells in the cell culture. In embodiments, the Treg cells are expanded from 1700 fold to more than 3000 fold compared to the original number or concentration of Treg cells in the cell culture. In embodiments, the Treg cells are expanded from 1800 fold to more than 3000 fold compared to the original number or concentration of Treg cells in the cell culture. In embodiments, the Treg cells are expanded from 1900 fold to more than 3000 fold compared to the original number or concentration of Treg cells in the cell culture. In embodiments, the Treg cells are expanded from 2000 fold to more than 3000 fold compared to the original number or concentration of Treg cells in the cell culture. In embodiments, the Treg cells are expanded from 2100 fold to more than 3000 fold compared to the original number or concentration of Treg cells in the cell culture. In embodiments, the Treg cells are expanded from 2200 fold to more than 3000 fold compared to the original number or concentration of Treg cells in the cell culture. In embodiments, the Treg cells are expanded from 2300 fold to more than 3000 fold compared to the original number or concentration of Treg cells in the cell culture. In embodiments, the Treg cells are expanded from 2400 fold to more than 3000 fold compared to the original number or concentration of Treg cells in the cell culture. In embodiments, the Treg cells are expanded from 2500 fold to more than 3000 fold compared to the original number or concentration of Treg cells in the cell culture. In embodiments, the Treg cells are expanded from 2600 fold to more than 3000 fold compared to the original number or concentration of Treg cells in the cell culture. In embodiments, the Treg cells are expanded from 2700 fold to more than 3000 fold compared to the original number or concentration of Treg cells in the cell culture. In embodiments, the Treg cells are expanded from 2800 fold to more than 3000 fold compared to the original number or concentration of Treg cells in the cell culture. In embodiments, the Treg cells are expanded from 2900 fold to more than 3000 fold compared to the original number or concentration of Treg cells in the cell culture.

In embodiments, the Treg cells are expanded from 100 fold to more than 2900 fold compared to the original number or concentration of Treg cells in the cell culture. In embodiments, the Treg cells are expanded from 100 fold to more than 2800 fold compared to the original number or concentration of Treg cells in the cell culture. In embodiments, the Treg cells are expanded from 100 fold to more than 2700 fold compared to the original number or concentration of Treg cells in the cell culture. In embodiments, the Treg cells are expanded from 100 fold to more than 2600 fold compared to the original number or concentration of Treg cells in the cell culture. In embodiments, the Treg cells are expanded from 100 fold to more than 2500 fold compared to the original number or concentration of Treg cells in the cell culture. In embodiments, the Treg cells are expanded from 100 fold to more than 2400 fold compared to the original number or concentration of Treg cells in the cell culture. In embodiments, the Treg cells are expanded from 100 fold to more than 2300 fold compared to the original number or concentration of Treg cells in the cell culture. In embodiments, the Treg cells are expanded from 100 fold to more than 2200 fold compared to the original number or concentration of Treg cells in the cell culture. In embodiments, the Treg cells are expanded from 100 fold to more than 2100 fold compared to the original number or concentration of Treg cells in the cell culture. In embodiments, the Treg cells are expanded from 100 fold to more than 2000 fold compared to the original number or concentration of Treg cells in the cell culture. In embodiments, the Treg cells are expanded from 100 fold to more than 1900 fold compared to the original number or concentration of Treg cells in the cell culture. In embodiments, the Treg cells are expanded from 100 fold to more than 1800 fold compared to the original number or concentration of Treg cells in the cell culture. In embodiments, the Treg cells are expanded from 100 fold to more than 1700 fold compared to the original number or concentration of Treg cells in the cell culture. In embodiments, the Treg cells are expanded from 100 fold to more than 1600 fold compared to the original number or concentration of Treg cells in the cell culture. In embodiments, the Treg cells are expanded from 100 fold to more than 1500 fold compared to the original number or concentration of Treg cells in the cell culture. In embodiments, the Treg cells are expanded from 100 fold to more than 1400 fold compared to the original number or concentration of Treg cells in the cell culture. In embodiments, the Treg cells are expanded from 100 fold to more than 1300 fold compared to the original number or concentration of Treg cells in the cell culture. In embodiments, the Treg cells are expanded from 100 fold to more than 1200 fold compared to the original number or concentration of Treg cells in the cell culture. In embodiments, the Treg cells are expanded from 100 fold to more than 1100 fold compared to the original number or concentration of Treg cells in the cell culture. In embodiments, the Treg cells are expanded from 100 fold to more than 1000 fold compared to the original number or concentration of Treg cells in the cell culture. In embodiments, the Treg cells are expanded from 100 fold to more than 900 fold compared to the original number or concentration of Treg cells in the cell culture. In embodiments, the Treg cells are expanded from 100 fold to more than 800 fold compared to the original number or concentration of Treg cells in the cell culture. In embodiments, the Treg cells are expanded from 100 fold to more than 700 fold compared to the original number or concentration of Treg cells in the cell culture. In embodiments, the Treg cells are expanded from 100 fold to more than 600 fold compared to the original number or concentration of Treg cells in the cell culture. In embodiments, the Treg cells are expanded from 100 fold to more than 500 fold compared to the original number or concentration of Treg cells in the cell culture. In embodiments, the Treg cells are expanded from 100 fold to more than 400 fold compared to the original number or concentration of Treg cells in the cell culture. In embodiments, the Treg cells are expanded from 100 fold to more than 300 fold compared to the original number or concentration of Treg cells in the cell culture. In embodiments, the Treg cells are expanded from 100 fold to more than 200 fold compared to the original number or concentration of Treg cells in the cell culture. In embodiments, the Treg cells are expanded from 100 fold, 200 fold, 300 fold, 400 fold, 500 fold, 600 fold, 700 fold, 800 fold, 900 fold, 1000 fold, 1100 fold, 1200 fold, 1300 fold, 1400 fold, 1500 fold, 1600 fold, 1700 fold, 1800 fold, 1900 fold, 2000 fold, 2100 fold, 2200 fold, 2300 fold, 2400 fold, 2500 fold, 2600 fold, 2700 fold, 2800 fold, 2900 fold, or 3000 fold, compared to the original number or concentration of Treg cells in the cell culture. In embodiments, the Treg cells are expanded 600 fold compared to the original number or concentration of Treg cells in the cell culture.

In embodiments, the plurality of Treg cells are expanded during the contacting with the plurality of recipient antigen presenting cells, thereby forming a population of recipient antigen-specific regulatory T cells. In embodiments, the recipient antigen-specific regulatory T cells are expanded, thereby forming a population of recipient antigen-specific regulatory T cells.

For the methods provided herein, in embodiments, the recipient antigen-specific Treg cells are expanded from 2 fold to more than 200 fold compared to the original number or concentration of recipient antigen-specific Treg cells in the cell culture. In embodiments, the recipient recipient antigen-specific Treg cells are expanded from 8 fold to more than 200 fold compared to the original number or concentration of recipient antigen-specific Treg cells in the cell culture. In embodiments, the recipient antigen-specific Treg cells are expanded from 16 fold to more than 200 fold compared to the original number or concentration of recipient antigen-specific Treg cells in the cell culture. In embodiments, the recipient antigen-specific Treg cells are expanded from 32 fold to more than 200 fold compared to the original number or concentration of recipient antigen-specific Treg cells in the cell culture. In embodiments, the recipient antigen-specific Treg cells are expanded from 40 fold to more than 200 fold compared to the original number or concentration of recipient antigen-specific Treg cells in the cell culture. In embodiments, the recipient antigen-specific Treg cells are expanded from 48 fold to more than 200 fold compared to the original number or concentration of recipient antigen-specific Treg cells in the cell culture. In embodiments, the recipient antigen-specific Treg cells are expanded from 56 fold to more than 200 fold compared to the original number or concentration of recipient antigen-specific Treg cells in the cell culture. In embodiments, the recipient antigen-specific Treg cells are expanded from 64 fold to more than 200 fold compared to the original number or concentration of recipient antigen-specific Treg cells in the cell culture. In embodiments, the recipient antigen-specific Treg cells are expanded from 72 fold to more than 200 fold compared to the original number or concentration of recipient antigen-specific Treg cells in the cell culture. In embodiments, the recipient antigen-specific Treg cells are expanded from 88 fold to more than 200 fold compared to the original number or concentration of recipient antigen-specific Treg cells in the cell culture. In embodiments, the recipient antigen-specific Treg cells are expanded from 98 fold to more than 200 fold compared to the original number or concentration of recipient antigen-specific Treg cells in the cell culture. In embodiments, the recipient antigen-specific Treg cells are expanded from 104 fold to more than 200 fold compared to the original number or concentration of recipient antigen-specific Treg cells in the cell culture. In embodiments, the recipient antigen-specific Treg cells are expanded from 112 fold to more than 200 fold compared to the original number or concentration of recipient antigen-specific Treg cells in the cell culture. In embodiments, the recipient antigen-specific Treg cells are expanded from 120 fold to more than 200 fold compared to the original number or concentration of recipient antigen-specific Treg cells in the cell culture. In embodiments, the recipient antigen-specific Treg cells are expanded from 128 fold to more than 200 fold compared to the original number or concentration of recipient antigen-specific Treg cells in the cell culture. In embodiments, the recipient antigen-specific Treg cells are expanded from 136 fold to more than 200 fold compared to the original number or concentration of recipient antigen-specific Treg cells in the cell culture. In embodiments, the recipient antigen-specific Treg cells are expanded from 144 fold to more than 200 fold compared to the original number or concentration of recipient antigen-specific Treg cells in the cell culture. In embodiments, the recipient antigen-specific Treg cells are expanded from 152 fold to more than 200 fold compared to the original number or concentration of recipient antigen-specific Treg cells in the cell culture. In embodiments, the recipient antigen-specific Treg cells are expanded from 160 fold to more than 200 fold compared to the original number or concentration of recipient antigen-specific Treg cells in the cell culture. In embodiments, the recipient antigen-specific Treg cells are expanded from 168 fold to more than 200 fold compared to the original number or concentration of recipient antigen-specific Treg cells in the cell culture. In embodiments, the recipient antigen-specific Treg cells are expanded from 176 fold to more than 200 fold compared to the original number or concentration of recipient antigen-specific Treg cells in the cell culture. In embodiments, the recipient antigen-specific Treg cells are expanded from 184 fold to more than 200 fold compared to the original number or concentration of recipient antigen-specific Treg cells in the cell culture. In embodiments, the recipient antigen-specific Treg cells are expanded from 192 fold to more than 200 fold compared to the original number or concentration of recipient antigen-specific Treg cells in the cell culture.

In embodiments, the recipient antigen-specific Treg cells are expanded from 2 fold to 192 fold compared to the original number or concentration of recipient antigen-specific Treg cells in the cell culture. In embodiments, the recipient antigen-specific Treg cells are expanded from 2 fold to 184 fold compared to the original number or concentration of recipient antigen-specific Treg cells in the cell culture. In embodiments, the recipient antigen-specific Treg cells are expanded from 2 fold to 176 fold compared to the original number or concentration of recipient antigen-specific Treg cells in the cell culture. In embodiments, the recipient antigen-specific Treg cells are expanded from 2 fold to 168 fold compared to the original number or concentration of recipient antigen-specific Treg cells in the cell culture. In embodiments, the recipient antigen-specific Treg cells are expanded from 2 fold to 160 fold compared to the original number or concentration of recipient antigen-specific Treg cells in the cell culture. In embodiments, the recipient antigen-specific Treg cells are expanded from 2 fold to 152 fold compared to the original number or concentration of recipient antigen-specific Treg cells in the cell culture. In embodiments, the recipient antigen-specific Treg cells are expanded from 2 fold to 144 fold compared to the original number or concentration of recipient antigen-specific Treg cells in the cell culture. In embodiments, the recipient antigen-specific Treg cells are expanded from 2 fold to 136 fold compared to the original number or concentration of recipient antigen-specific Treg cells in the cell culture. In embodiments, the recipient antigen-specific Treg cells are expanded from 2 fold to 128 fold compared to the original number or concentration of recipient antigen-specific Treg cells in the cell culture. In embodiments, the recipient antigen-specific Treg cells are expanded from 2 fold to 120 fold compared to the original number or concentration of recipient antigen-specific Treg cells in the cell culture. In embodiments, the recipient antigen-specific Treg cells are expanded from 2 fold to 112 fold compared to the original number or concentration of recipient antigen-specific Treg cells in the cell culture. In embodiments, the recipient antigen-specific Treg cells are expanded from 2 fold to 104 fold compared to the original number or concentration of recipient antigen-specific Treg cells in the cell culture. In embodiments, the recipient antigen-specific Treg cells are expanded from 2 fold to 88 fold compared to the original number or concentration of recipient antigen-specific Treg cells in the cell culture. In embodiments, the recipient antigen-specific Treg cells are expanded from 2 fold to 80 fold compared to the original number or concentration of recipient antigen-specific Treg cells in the cell culture. In embodiments, the recipient antigen-specific Treg cells are expanded from 2 fold to 72 fold compared to the original number or concentration of recipient antigen-specific Treg cells in the cell culture. In embodiments, the recipient antigen-specific Treg cells are expanded from 2 fold to 64 fold compared to the original number or concentration of recipient antigen-specific Treg cells in the cell culture. In embodiments, the recipient antigen-specific Treg cells are expanded from 2 fold to 56 fold compared to the original number or concentration of recipient antigen-specific Treg cells in the cell culture. In embodiments, the recipient antigen-specific Treg cells are expanded from 2 fold to 48 fold compared to the original number or concentration of recipient antigen-specific Treg cells in the cell culture. In embodiments, the recipient antigen-specific Treg cells are expanded from 2 fold to 40 fold compared to the original number or concentration of recipient antigen-specific Treg cells in the cell culture. In embodiments, the recipient antigen-specific Treg cells are expanded from 2 fold to 32 fold compared to the original number or concentration of recipient antigen-specific Treg cells in the cell culture. In embodiments, the recipient antigen-specific Treg cells are expanded from 2 fold to 24 fold compared to the original number or concentration of recipient antigen-specific Treg cells in the cell culture. In embodiments, the recipient antigen-specific Treg cells are expanded from 2 fold to 16 fold compared to the original number or concentration of recipient antigen-specific Treg cells in the cell culture. In embodiments, the recipient antigen-specific Treg cells are expanded from 2 fold to 8 fold compared to the original number or concentration of recipient antigen-specific Treg cells in the cell culture. In embodiments, the recipient antigen-specific Treg cells are expanded from 2 fold, 8 fold, 16 fold, 24 fold, 32 fold, 40 fold, 48 fold, 56 fold, 64 fold, 72 fold, 80 fold, 88 fold, 96 fold, 104 fold, 112 fold, 120 fold, 128 fold, 136 fold, 144 fold, 152 fold, 160 fold, 168 fold, 176 fold, 184 fold, 192 fold, or 200 fold compared to the original number or concentration of recipient antigen-specific Treg cells in the cell culture. In embodiments, the recipient antigen-specific Treg cells are expanded 100 fold compared to the original number or concentration of recipient antigen-specific Treg cells in the cell culture.

For the methods provided herein, in embodiments, the recipient antigen-specific Treg cells are expanded from 100 fold to more than 3000 fold compared to the original number or concentration of recipient antigen-specific Treg cells in the cell culture. In embodiments, the recipient antigen-specific Treg cells are expanded from 200 fold to more than 3000 fold compared to the original number or concentration of recipient antigen-specific Treg cells in the cell culture. In embodiments, the recipient antigen-specific Treg cells are expanded from 300 fold to more than 3000 fold compared to the original number or concentration of recipient antigen-specific Treg cells in the cell culture. In embodiments, the recipient antigen-specific Treg cells are expanded from 400 fold to more than 3000 fold compared to the original number or concentration of recipient antigen-specific Treg cells in the cell culture. In embodiments, the recipient antigen-specific Treg cells are expanded from 500 fold to more than 3000 fold compared to the original number or concentration of recipient antigen-specific Treg cells in the cell culture. In embodiments, the recipient antigen-specific Treg cells are expanded from 600 fold to more than 3000 fold compared to the original number or concentration of recipient antigen-specific Treg cells in the cell culture. In embodiments, the recipient antigen-specific Treg cells are expanded from 700 fold to more than 3000 fold compared to the original number or concentration of recipient antigen-specific Treg cells in the cell culture. In embodiments, the recipient antigen-specific Treg cells are expanded from 800 fold to more than 3000 fold compared to the original number or concentration of recipient antigen-specific Treg cells in the cell culture. In embodiments, the recipient antigen-specific Treg cells are expanded from 900 fold to more than 3000 fold compared to the original number or concentration of recipient antigen-specific Treg cells in the cell culture. In embodiments, the recipient antigen-specific Treg cells are expanded from 1000 fold to more than 3000 fold compared to the original number or concentration of recipient antigen-specific Treg cells in the cell culture. In embodiments, the recipient antigen-specific Treg cells are expanded from 1100 fold to more than 3000 fold compared to the original number or concentration of recipient antigen-specific Treg cells in the cell culture. In embodiments, the recipient antigen-specific Treg cells are expanded from 1200 fold to more than 3000 fold compared to the original number or concentration of recipient antigen-specific Treg cells in the cell culture. In embodiments, the recipient antigen-specific Treg cells are expanded from 1300 fold to more than 3000 fold compared to the original number or concentration of recipient antigen-specific Treg cells in the cell culture. In embodiments, the recipient antigen-specific Treg cells are expanded from 1400 fold to more than 3000 fold compared to the original number or concentration of recipient antigen-specific Treg cells in the cell culture. In embodiments, the recipient antigen-specific Treg cells are expanded from 1500 fold to more than 3000 fold compared to the original number or concentration of recipient antigen-specific Treg cells in the cell culture. In embodiments, the recipient antigen-specific Treg cells are expanded from 1600 fold to more than 3000 fold compared to the original number or concentration of recipient antigen-specific Treg cells in the cell culture. In embodiments, the recipient antigen-specific Treg cells are expanded from 1700 fold to more than 3000 fold compared to the original number or concentration of recipient antigen-specific Treg cells in the cell culture. In embodiments, the recipient antigen-specific Treg cells are expanded from 1800 fold to more than 3000 fold compared to the original number or concentration of recipient antigen-specific Treg cells in the cell culture. In embodiments, the recipient antigen-specific Treg cells are expanded from 1900 fold to more than 3000 fold compared to the original number or concentration of recipient antigen-specific Treg cells in the cell culture. In embodiments, the recipient antigen-specific Treg cells are expanded from 2000 fold to more than 3000 fold compared to the original number or concentration of recipient antigen-specific Treg cells in the cell culture. In embodiments, the recipient antigen-specific Treg cells are expanded from 2100 fold to more than 3000 fold compared to the original number or concentration of recipient antigen-specific Treg cells in the cell culture. In embodiments, the recipient antigen-specific Treg cells are expanded from 2200 fold to more than 3000 fold compared to the original number or concentration of recipient antigen-specific Treg cells in the cell culture. In embodiments, the recipient antigen-specific Treg cells are expanded from 2300 fold to more than 3000 fold compared to the original number or concentration of recipient antigen-specific Treg cells in the cell culture. In embodiments, the recipient antigen-specific Treg cells are expanded from 2400 fold to more than 3000 fold compared to the original number or concentration of recipient antigen-specific Treg cells in the cell culture. In embodiments, the recipient antigen-specific Treg cells are expanded from 2500 fold to more than 3000 fold compared to the original number or concentration of recipient antigen-specific Treg cells in the cell culture. In embodiments, the recipient antigen-specific Treg cells are expanded from 2600 fold to more than 3000 fold compared to the original number or concentration of recipient antigen-specific Treg cells in the cell culture. In embodiments, the recipient antigen-specific Treg cells are expanded from 2700 fold to more than 3000 fold compared to the original number or concentration of recipient antigen-specific Treg cells in the cell culture. In embodiments, the recipient antigen-specific Treg cells are expanded from 2800 fold to more than 3000 fold compared to the original number or concentration of recipient antigen-specific Treg cells in the cell culture. In embodiments, the recipient antigen-specific Treg cells are expanded from 2900 fold to more than 3000 fold compared to the original number or concentration of recipient antigen-specific Treg cells in the cell culture.

In embodiments, the recipient antigen-specific Treg cells are expanded from 100 fold to more than 2900 fold compared to the original number or concentration of recipient antigen-specific Treg cells in the cell culture. In embodiments, the recipient antigen-specific Treg cells are expanded from 100 fold to more than 2800 fold compared to the original number or concentration of recipient antigen-specific Treg cells in the cell culture. In embodiments, the recipient antigen-specific Treg cells are expanded from 100 fold to more than 2700 fold compared to the original number or concentration of recipient antigen-specific Treg cells in the cell culture. In embodiments, the recipient antigen-specific Treg cells are expanded from 100 fold to more than 2600 fold compared to the original number or concentration of recipient antigen-specific Treg cells in the cell culture. In embodiments, the recipient antigen-specific Treg cells are expanded from 100 fold to more than 2500 fold compared to the original number or concentration of recipient antigen-specific Treg cells in the cell culture. In embodiments, the recipient antigen-specific Treg cells are expanded from 100 fold to more than 2400 fold compared to the original number or concentration of recipient antigen-specific Treg cells in the cell culture. In embodiments, the recipient antigen-specific Treg cells are expanded from 100 fold to more than 2300 fold compared to the original number or concentration of recipient antigen-specific Treg cells in the cell culture. In embodiments, the recipient antigen-specific Treg cells are expanded from 100 fold to more than 2200 fold compared to the original number or concentration of recipient antigen-specific Treg cells in the cell culture. In embodiments, the recipient antigen-specific Treg cells are expanded from 100 fold to more than 2100 fold compared to the original number or concentration of recipient antigen-specific Treg cells in the cell culture. In embodiments, the recipient antigen-specific Treg cells are expanded from 100 fold to more than 2000 fold compared to the original number or concentration of recipient antigen-specific Treg cells in the cell culture. In embodiments, the recipient antigen-specific recipient antigen-specific Treg cells are expanded from 100 fold to more than 1900 fold compared to the original number or concentration of recipient antigen-specific Treg cells in the cell culture. In embodiments, the recipient antigen-specific Treg cells are expanded from 100 fold to more than 1800 fold compared to the original number or concentration of recipient antigen-specific Treg cells in the cell culture. In embodiments, the recipient antigen-specific Treg cells are expanded from 100 fold to more than 1700 fold compared to the original number or concentration of recipient antigen-specific Treg cells in the cell culture. In embodiments, the recipient antigen-specific Treg cells are expanded from 100 fold to more than 1600 fold compared to the original number or concentration of recipient antigen-specific Treg cells in the cell culture. In embodiments, the recipient antigen-specific Treg cells are expanded from 100 fold to more than 1500 fold compared to the original number or concentration of recipient antigen-specific Treg cells in the cell culture. In embodiments, the recipient antigen-specific Treg cells are expanded from 100 fold to more than 1400 fold compared to the original number or concentration of recipient antigen-specific Treg cells in the cell culture. In embodiments, the recipient antigen-specific Treg cells are expanded from 100 fold to more than 1300 fold compared to the original number or concentration of recipient antigen-specific Treg cells in the cell culture. In embodiments, the recipient antigen-specific Treg cells are expanded from 100 fold to more than 1200 fold compared to the original number or concentration of recipient antigen-specific Treg cells in the cell culture. In embodiments, the recipient antigen-specific Treg cells are expanded from 100 fold to more than 1100 fold compared to the original number or concentration of recipient antigen-specific Treg cells in the cell culture. In embodiments, the recipient antigen-specific Treg cells are expanded from 100 fold to more than 1000 fold compared to the original number or concentration of recipient antigen-specific Treg cells in the cell culture. In embodiments, the recipient antigen-specific Treg cells are expanded from 100 fold to more than 900 fold compared to the original number or concentration of recipient antigen-specific Treg cells in the cell culture. In embodiments, the recipient antigen-specific Treg cells are expanded from 100 fold to more than 800 fold compared to the original number or concentration of recipient antigen-specific Treg cells in the cell culture. In embodiments, the recipient antigen-specific Treg cells are expanded from 100 fold to more than 700 fold compared to the original number or concentration of recipient antigen-specific Treg cells in the cell culture. In embodiments, the recipient antigen-specific Treg cells are expanded from 100 fold to more than 600 fold compared to the original number or concentration of recipient antigen-specific Treg cells in the cell culture. In embodiments, the recipient antigen-specific Treg cells are expanded from 100 fold to more than 500 fold compared to the original number or concentration of recipient antigen-specific Treg cells in the cell culture. In embodiments, the recipient antigen-specific Treg cells are expanded from 100 fold to more than 400 fold compared to the original number or concentration of recipient antigen-specific Treg cells in the cell culture. In embodiments, the recipient antigen-specific Treg cells are expanded from 100 fold to more than 300 fold compared to the original number or concentration of recipient antigen-specific Treg cells in the cell culture. In embodiments, the recipient antigen-specific Treg cells are expanded from 100 fold to more than 200 fold compared to the original number or concentration of recipient antigen-specific Treg cells in the cell culture. In embodiments, the recipient antigen-specific Treg cells are expanded from 100 fold, 200 fold, 300 fold, 400 fold, 500 fold, 600 fold, 700 fold, 800 fold, 900 fold, 1000 fold, 1100 fold, 1200 fold, 1300 fold, 1400 fold, 1500 fold, 1600 fold, 1700 fold, 1800 fold, 1900 fold, 2000 fold, 2100 fold, 2200 fold, 2300 fold, 2400 fold, 2500 fold, 2600 fold, 2700 fold, 2800 fold, 2900 fold, or 3000 fold, compared to the original number or concentration of recipient antigen-specific Treg cells in the cell culture. In embodiments, the recipient antigen-specific Treg cells are expanded 600 fold compared to the original number or concentration of recipient antigen-specific Treg cells in the cell culture.

In embodiments, the recipient antigen-specific Treg cells are expanded for 0.5 days to 10 days. In embodiments, the recipient antigen-specific Treg cells are expanded for 1 days to 10 days. In embodiments, the recipient antigen-specific Treg cells are expanded for 1.5 days to 10 days. In embodiments, the recipient antigen-specific Treg cells are expanded for 2 days to 10 days. In embodiments, the recipient antigen-specific Treg cells are expanded for 2.5 days to 10 days. In embodiments, the recipient antigen-specific Treg cells are expanded for 3 days to 10 days. In embodiments, the recipient antigen-specific Treg cells are expanded for 3.5 days to 10 days. In embodiments, the recipient antigen-specific Treg cells are expanded for 4 days to 10 days. In embodiments, the recipient antigen-specific Treg cells are expanded for 4.5 days to 10 days. In embodiments, the recipient antigen-specific Treg cells are expanded for 5 days to 10 days. In embodiments, the recipient antigen-specific Treg cells are expanded for 5.5 days to 10 days. In embodiments, the recipient antigen-specific Treg cells are expanded for 6 days to 10 days. In embodiments, the recipient antigen-specific Treg cells are expanded for 6.5 days to 10 days. In embodiments, the recipient antigen-specific Treg cells are expanded for 7 days to 10 days. In embodiments, the recipient antigen-specific Treg cells are expanded for 7.5 days to 10 days. In embodiments, the recipient antigen-specific Treg cells are expanded for 8 days to 10 days. In embodiments, the recipient antigen-specific Treg cells are expanded for 8.5 days to 10 days. In embodiments, the recipient antigen-specific Treg cells are expanded for 9 days to 10 days. In embodiments, the recipient antigen-specific Treg cells are expanded for 9.5 days to 10 days. In embodiments, the recipient antigen-specific Treg cells are expanded for 0.5 days to 9.5 days. In embodiments, the recipient antigen-specific Treg cells are expanded for 0.5 days to 9 days. In embodiments, the recipient antigen-specific Treg cells are expanded for 0.5 days to 8.5 days. In embodiments, the recipient antigen-specific Treg cells are expanded for 0.5 days to 8 days. In embodiments, the recipient antigen-specific Treg cells are expanded for 0.5 days to 7.5 days. In embodiments, the recipient antigen-specific Treg cells are expanded for 0.5 days to 7 days. In embodiments, the recipient antigen-specific Treg cells are expanded for 0.5 days to 6.5 days. In embodiments, the recipient antigen-specific Treg cells are expanded for 0.5 days to 6 days. In embodiments, the recipient antigen-specific Treg cells are expanded for 0.5 days to 5.5 days. In embodiments, the recipient antigen-specific Treg cells are expanded for 0.5 days to 5 days. In embodiments, the recipient antigen-specific Treg cells are expanded for 0.5 days to 4.5 days. In embodiments, the recipient antigen-specific Treg cells are expanded for 0.5 days to 4 days. In embodiments, the recipient antigen-specific Treg cells are expanded for 0.5 days to 3.5 days. In embodiments, the recipient antigen-specific Treg cells are expanded for 0.5 days to 3 days. In embodiments, the recipient antigen-specific Treg cells are expanded for 0.5 days to 2.5 days. In embodiments, the recipient antigen-specific Treg cells are expanded for 0.5 days to 2 days. In embodiments, the recipient antigen-specific Treg cells are expanded for 0.5 days to 1.5 days. In embodiments, the recipient antigen-specific Treg cells are expanded for 0.5 days to 1 day. In embodiments, the recipient antigen-specific Treg cells are expanded for 0.5 days, 1 day, 1.5 days, 2 days, 2.5 days, 3 days, 3.5 days, 4 days, 4.5 days, 5 days, 5.5 days, 6 days, 6.5 days, 7 days, 7.5 days, 8 days, 8.5 days, 9 days, 9.5 days, or 10 days.

In embodiments, the method further includes isolating the recipient antigen-specific Treg cells. The recipient antigen-specific Treg cells can be isolated using any method known in the art. For example, the genetically modified cells can be isolated by FACS, antibody-based immunological methods (e.g. ELISA, etc.), magnetic-based cell sorting, etc. After isolating the recipient antigen-specific Treg cells, the purity of the antigen-specific Treg cells may be 50%, 55%, 60%, 65%, 70%, 75%, 80% 85%, 90%, 95%, or 100%.

In embodiments, the recipient antigen presenting cells present pre-processed recipient antigen which are recognized as foreign by immune cells (e.g. donor immune cells) from the tissue transplant. In embodiments, recognition of said recipient antigen by immune cells from the tissue transplant increases proliferation and/or activation of the immune cells, thereby causing GvHD. Thus, in embodiments, the plurality of recipient antigen presenting cells are taken from the subject when the subject has graft-versus-host disease. In embodiments, the graft-versus-host disease is chronic graft-versus-host disease. In embodiments, the subject has graft-versus-host-disease when the subject exhibits one or more symptoms of graft-versus-host disease, as provided herein. In embodiments, the subject has antigen presenting cells including recipient antigens that induce an immune response from immune cells present in the tissue transplant.

In embodiments, the subject is treated for graft-versus-host disease with steroids, corticosteroids, and/or other therapeutic treatments which may damage the immune system of the subject. Thus, in embodiments, the plurality of recipient antigen presenting cells are taken from the subject prior to administration of a graft-versus-host disease therapeutic treatment. In embodiments, the graft-versus-host disease therapeutic treatment includes a corticosteroid and/or immunosuppressive compound. In embodiments, the graft-versus-host disease therapeutic treatment includes a corticosteroid. In embodiments, the graft-versus-host disease therapeutic treatment includes an immunosuppressive compound. In embodiments, the immunosuppressive compound is Methotrexate, Cyclosporine, Tacrolimus, Mycophenolate mofetil, Sirolimus, Antithymocyte globulin, Alemtuzumab, Cyclophosphamide or Inolimomab. In embodiments, the immunosuppressive compound is Methotrexate. In embodiments, the immunosuppressive compound is Cyclosporine. In embodiments, the immunosuppressive compound is Tacrolimus. In embodiments, the immunosuppressive compound is Mycophenolate mofetil. In embodiments, the immunosuppressive compound is Antithymocyte globulin. In embodiments, the immunosuppressive compound is Alemtuzumab. In embodiments, the immunosuppressive compound is Cyclophosphamide. In embodiments, the immunosuppressive compound is Inolimomab.

In embodiments, the corticosteroid is prednisone, methylprednisolone, dexamethasone, beclomethasone or budesonide. In embodiments, the corticosteroid is prednisone. In embodiments, the corticosteroid is methylprednisolone. In embodiments, the corticosteroid is dexamethasone. In embodiments, the corticosteroid is beclomethasone. In embodiments, the corticosteroid is budesonide.

In embodiments, the plurality of recipient antigen presenting cells include dendritic cells. In embodiments, the plurality of recipient antigen presenting cells include B cells. In embodiments the plurality of recipient APCs include DC to B cells at a ratio of 0.1:1 (DC:B cells), 0.2:1, 0.3:1, 0.4:1, 0.5:1, 0.6:1, 0.7:1, 0.8:1, 0.9:1, 1:1, 1:0.9, 1:0.8, 1:0.7, 1:0.6, 1:0.5, 1:0.4, 1:0.3, 1:0.2, or 1:0.1. In embodiments the plurality of recipient APCs include DC to B cells at a ratio of 1:1.

In embodiments, CD28 inhibitor compound is a CD80/CD86 antagonist. In embodiments, the CD28 inhibitor compound is abatacept or belatacept. In embodiments, the CD28 inhibitor compound is abatacept. In embodiments, the CD28 inhibitor compound is belatacept.

For the methods provided herein, in embodiments, the concentration of the CD28 inhibitor compound is from 1 ug/ml to 100 ug/ml. In embodiments, the concentration of the CD28 inhibitor compound is from 5 ug/ml to 100 ug/ml. In embodiments, the concentration of the CD28 inhibitor compound is from 10 ug/ml to 100 ug/ml. In embodiments, the concentration of the CD28 inhibitor compound is from 15 ug/ml to 100 ug/ml. In embodiments, the concentration of the CD28 inhibitor compound is from 20 ug/ml to 100 ug/ml. In embodiments, the concentration of the CD28 inhibitor compound is from 25 ug/ml to 100 ug/ml. In embodiments, the concentration of the CD28 inhibitor compound is from 30 ug/ml to 100 ug/ml. In embodiments, the concentration of the CD28 inhibitor compound is from 35 ug/ml to 100 ug/ml. In embodiments, the concentration of the CD28 inhibitor compound is from 40 ug/ml to 100 ug/ml. In embodiments, the concentration of the CD28 inhibitor compound is from 45 ug/ml to 100 ug/ml. In embodiments, the concentration of the CD28 inhibitor compound is from 50 ug/ml to 100 ug/ml. In embodiments, the concentration of the CD28 inhibitor compound is from 55 ug/ml to 100 ug/ml. In embodiments, the concentration of the CD28 inhibitor compound is from 60 ug/ml to 100 ug/ml. In embodiments, the concentration of the CD28 inhibitor compound is from 65 ug/ml to 100 ug/ml. In embodiments, the concentration of the CD28 inhibitor compound is from 70 ug/ml to 100 ug/ml. In embodiments, the concentration of the CD28 inhibitor compound is from 75 ug/ml to 100 ug/ml. In embodiments, the concentration of the CD28 inhibitor compound is from 80 ug/ml to 100 ug/ml. In embodiments, the concentration of the CD28 inhibitor compound is from 85 ug/ml to 100 ug/ml. In embodiments, the concentration of the CD28 inhibitor compound is from 90 ug/ml to 100 ug/ml. In embodiments, the concentration of the CD28 inhibitor compound is from 95 ug/ml to 100 ug/ml.

For the methods provided herein, in embodiments, the concentration of the CD28 inhibitor compound is from 1 ug/ml to 95 ug/ml. For the methods provided herein, in embodiments, the concentration of the CD28 inhibitor compound is from 1 ug/ml to 90 ug/ml. For the methods provided herein, in embodiments, the concentration of the CD28 inhibitor compound is from 1 ug/ml to 85 ug/ml. For the methods provided herein, in embodiments, the concentration of the CD28 inhibitor compound is from 1 ug/ml to 80 ug/ml. For the methods provided herein, in embodiments, the concentration of the CD28 inhibitor compound is from 1 ug/ml to 75 ug/ml. For the methods provided herein, in embodiments, the concentration of the CD28 inhibitor compound is from 1 ug/ml to 70 ug/ml. For the methods provided herein, in embodiments, the concentration of the CD28 inhibitor compound is from 1 ug/ml to 65 ug/ml. For the methods provided herein, in embodiments, the concentration of the CD28 inhibitor compound is from 1 ug/ml to 60 ug/ml. For the methods provided herein, in embodiments, the concentration of the CD28 inhibitor compound is from 1 ug/ml to 55 ug/ml. For the methods provided herein, in embodiments, the concentration of the CD28 inhibitor compound is from 1 ug/ml to 50 ug/ml. For the methods provided herein, in embodiments, the concentration of the CD28 inhibitor compound is from 1 ug/ml to 45 ug/ml. For the methods provided herein, in embodiments, the concentration of the CD28 inhibitor compound is from 1 ug/ml to 40 ug/ml. For the methods provided herein, in embodiments, the concentration of the CD28 inhibitor compound is from 1 ug/ml to 35 ug/ml. For the methods provided herein, in embodiments, the concentration of the CD28 inhibitor compound is from 1 ug/ml to 30 ug/ml. For the methods provided herein, in embodiments, the concentration of the CD28 inhibitor compound is from 1 ug/ml to 25 ug/ml. For the methods provided herein, in embodiments, the concentration of the CD28 inhibitor compound is from 1 ug/ml to 20 ug/ml. For the methods provided herein, in embodiments, the concentration of the CD28 inhibitor compound is from 1 ug/ml to 15 ug/ml. For the methods provided herein, in embodiments, the concentration of the CD28 inhibitor compound is from 1 ug/ml to 10 ug/ml. For the methods provided herein, in embodiments, the concentration of the CD28 inhibitor compound is from 1 ug/ml to 5 ug/ml. For the methods provided herein, in embodiments, the concentration of the CD28 inhibitor compound is from 1 ug/ml, 5 ug/ml, 10 ug/mL, 15 ug/ml, 20 ug/mL, 25 ug/ml, 30 ug/mL, 35 ug/ml, 40 ug/mL, 45 ug/ml, 50 ug/mL, 55 ug/ml, 60 ug/mL, 65 ug/ml, 70 ug/mL, 75 ug/ml, 80 ug/mL, 85 ug/ml, 90 ug/mL, 95 ug/ml, or 100 ug/mL. In embodiments, the concentration of the CD28 inhibitor compound is 40 ug/ml.

For the methods provided herein, in embodiments, recipient antigen-specific regulatory T cells are formed by a method including: (a) expanding regulatory T cells in vitro, thereby forming a plurality of regulatory T cells; (b) contacting a plurality of recipient antigen presenting cells with recipient antigen in vitro, thereby forming a plurality of activated recipient antigen presenting cells; and (c) contacting the plurality of regulatory T cells with said plurality of activated recipient antigen presenting cells in the presence of a CD28 inhibitor compound, thereby forming the recipient antigen-specific regulatory T cells. In embodiments, step (c) further includes contacting the plurality of regulatory T cells with a second plurality of activated recipient antigen presenting cells. In embodiments, the plurality of recipient antigen presenting cells are taken from the subject's blood. In embodiments, the plurality of recipient antigen presenting cells are taken from the subject's tissue transplant.

In embodiments, the plurality of recipient antigen presenting cells are taken from the subject when the subject has graft-versus-host disease. In embodiments, the plurality of recipient antigen presenting cells are taken from the subject prior to administration of a graft-versus-host disease therapeutic treatment. In embodiments, the graft-versus-host disease therapeutic treatment includes a corticosteroid and/or immunosuppressive compound. In embodiments, the immunosuppressive compound is Methotrexate, Cyclosporine, Tacrolimus, Mycophenolate mofetil, Sirolimus, Antithymocyte globulin, Alemtuzumab, Cyclophosphamide or Inolimomab. In embodiments, the corticosteroid is prednisone, methylprednisolone, dexamethasone, beclomethasone or budesonide.

In embodiments, the plurality of recipient antigen presenting cells include dendritic cells. In embodiments, the plurality of recipient antigen presenting cells include B cells. In embodiments, the CD28 inhibitor compound is a CD80/CD86 antagonist. In embodiments, the CD28 inhibitor compound is abatacept or belatacept.

In embodiments, the plurality of Treg cells are expanded in the presence of activated recipient antigen presenting cells, thereby forming a population of recipient antigen-specific regulatory T cells.

Treg cells are able to suppress activation of the immune system, such cells can be used to treat an individual having a disease for which suppression of the immune system is desirable. Compositions of the present invention are particularly useful for treating autoimmune diseases. For example, Treg cells can be used to treat or prevent a disease or condition such as graft vs. host disease (GVHD) (e.g., after a bone marrow transplantation), allograft rejection following tissue transplantation, and the like. Thus, in an aspect is provided a method to treating an individual in need of such treatment, the method including administering a composition comprising antigen-specific expanded Treg cells. In embodiments, compositions are produced by culturing isolated cells comprising an initial population of regulatory T-cells, in the presence of an agent to expand the initial regulatory T-cells. In embodiments, the agent may be a drug, antibody, cell, cell product or cytokine. The agent may be, for example, rapamycin, antigen presenting cell, IL2, IL15, anti-CD3 antibody, or CD28 co-stimulation blockade such as belatacept.

In embodiments, the subject has been diagnosed with cancer or an immune disease. In embodiments, compositions of the present invention are administered to an individual at risk for developing cancer or an immune disease. Such risk can be due to, for example, genetic factors or exposure to environmental factors.

Provided herein are methods of treating or preventing graft-versus-host disease in a subject in need thereof, the method comprising administering to the subject a therapeutically effective amount of recipient antigen-specific regulatory T cell in the embodiments disclosed above; thereby treating or preventing graft-versus host disease in the subject.

Compositions of the present invention are administered using any known route used to administer therapeutic compositions, so long as such administration results in alleviation of symptoms of immune-mediated disease. Acceptable protocols by which to administer compositions of the present invention in an effective manner can vary according to individual dose size, number of doses, frequency of dose administration, and mode of administration. Determination of such protocols can be accomplished by those skilled in the art.

Also included in the present invention are kits useful for practicing the disclosed methods of the present invention. Thus, in an aspect is provided a kit for producing a population of cells that is enriched for stable, regulatory T-cells (Treg cells), the kit including (i) reagents of the present invention and ii) instructions for using the kit. Kits of the present invention can also include various reagents, such as buffers, necessary to practice the methods of the invention, as known in the art. Such reagents and buffers may be useful for establishing conditions appropriate for expanding isolated cells into enriched populations of Treg cells. Thus, such regents may include things such as, for example, tissue culture media and immunoregulatory molecules such as IL-2.

### Methods of Making Antigen-Specific T Regulatory Cells

Applicants have discovered methods for making recipient antigen-specific regulatory T cells, which can be used for treating graft-versus host disease. The methods provided herein including embodiments thereof bypass issues associated with immune cell preparation, including poor expansion of recipient APCs. For example, the methods provided herein include taking recipient APCs from the subject prior to administration of GvHD treatments to said subject. In another instance, the methods provided herein bypass unavailability of donor Treg cells by taking said donor Treg cells within a certain time frame of the tissue donation procedure. In embodiments, the methods provided herein generate viable recipient antigen-specific Treg cells, as measured by the expression levels of IL-10, IFN-γ, and/or FoxP3 in the resultant recipient antigen-specific Treg cells. Thus, in an aspect is provided a method of forming recipient antigen-specific regulatory T cells, the method including: (a) expanding regulatory T cells in vitro, wherein the regulatory T cells are from a donor of a tissue transplant, thereby forming a plurality of regulatory T cells; and (b) contacting the plurality of regulatory T cells with a plurality of recipient antigen presenting cells and a CD28 inhibitor compound in vitro, wherein the plurality of recipient antigen presenting cells is from a subject who has received the tissue transplant, thereby forming the recipient antigen-specific regulatory T cells.

In embodiments, step (b) further includes contacting the plurality of regulatory T cells with a second plurality of recipient antigen presenting cells. In embodiments, the plurality of recipient antigen presenting cells is taken from the subject's blood. In embodiments, the second plurality of recipient antigen presenting cells is taken from the subject's blood. In embodiments, the plurality of recipient antigen presenting cells is taken from the subject's tissue transplant. In embodiments, the second plurality of recipient antigen presenting cells is taken from the subject's tissue transplant.

In embodiments, the plurality of recipient antigen presenting cells is taken from the subject when the subject has graft-versus-host disease. In embodiments, the plurality of recipient antigen presenting cells is taken from the subject prior to administration of a graft-versus-host disease therapeutic treatment. In embodiments, the graft-versus-host disease therapeutic treatment includes a corticosteroid and/or immunosuppressive compound. In embodiments, the immunosuppressive compound is Methotrexate, Cyclosporine, Tacrolimus, Mycophenolate mofetil, Sirolimus, Antithymocyte globulin, Alemtuzumab, Cyclophosphamide or Inolimomab. In embodiments, the corticosteroid is prednisone, methylprednisolone, dexamethasone, beclomethasone or budesonide.

In embodiments, the plurality of recipient antigen presenting cells include dendritic cells. In embodiments, the plurality of recipient antigen presenting cells include B cells.

In embodiments, the CD28 inhibitor compound is a CD80/CD86 antagonist. In embodiments, the CD28 inhibitor compound is abatacept or belatacept.

In an aspect is provided a method of forming recipient antigen-specific regulatory T cells, the method including: (a) expanding regulatory T cells in vitro, wherein the regulatory T cells are from a donor of a tissue transplant, thereby forming a plurality of regulatory T cells; (b) contacting a plurality of recipient antigen presenting cells with a recipient antigen in vitro, wherein the plurality of recipient antigen presenting cells is from a subject who has received the tissue transplant, thereby forming a plurality of activated recipient antigen presenting cells; and (c) contacting the plurality of regulatory T cells with the plurality of activated recipient antigen presenting cells and a CD28 inhibitor compound in vitro, thereby forming the recipient antigen-specific regulatory T cells. In embodiments, step (c) further includes contacting the plurality of regulatory T cells with a second plurality of recipient antigen presenting cells. In embodiments, the plurality of recipient antigen presenting cells is taken from the subject's blood. In embodiments, the second plurality of recipient antigen presenting cells is taken from the subject's blood. In embodiments, the plurality of recipient antigen presenting cells is taken from the subject's tissue transplant. In embodiments, the second plurality of recipient antigen presenting cells is taken from the subject's tissue transplant.

In embodiments, the plurality of recipient antigen presenting cells is taken from the subject when the subject has graft-versus-host disease. In embodiments, the plurality of recipient antigen presenting cells is taken from the subject prior to administration of a graft-versus-host disease therapeutic treatment. In embodiments, the graft-versus-host disease therapeutic treatment includes a corticosteroid and/or immunosuppressive compound. In embodiments, the immunosuppressive compound is Methotrexate, Cyclosporine, Tacrolimus, Mycophenolate mofetil, Sirolimus, Antithymocyte globulin, Alemtuzumab, Cyclophosphamide or Inolimomab. In embodiments, the corticosteroid is prednisone, methylprednisolone, dexamethasone, beclomethasone or budesonide.

In embodiments, the plurality of recipient antigen presenting cells include dendritic cells. In embodiments, the plurality of recipient antigen presenting cells include B cells.

In embodiments, the CD28 inhibitor compound is a CD80/CD86 antagonist. In embodiments, the CD28 inhibitor compound is abatacept or belatacept.

Provided herein are methods of generating and expanding regulatory T cells (Treg cells) for use in immunotherapy. In particular, provided herein are robust approaches for the expansion of donor-sourced Treg cells that are recipient antigen-specific. Treg cells produced by the methods provided herein including embodiments thereof are suitable for the induction and/or maintenance of immunologic tolerance (e.g. having immunosuppressive properties or maintains non-responsiveness of the immune system to an antigen). Immunologic tolerance is useful, for example, in recipients of allogeneic transplants.

Methods of the present invention can generally be practiced by culturing isolated cells that comprise a population of T-cells, in the presence of one or more agents to expand at least a portion of the T-cell population. In embodiments, the isolated cells comprise an initial population of regulatory T-cells, and the culture conditions result in expansion of antigen-specific regulatory T-cells. The result of such methods is an expanded population of cells that can be used to treat immune disease. In embodiments, the disease is graft-versus-host disease.

Provided herein are methods for isolating regulatory T cells. Such cells can be obtained as a sample from an animal, including humans, or they can be obtained from cells in culture. Examples of cell samples useful for practicing the present invention include, but are not limited to, blood samples, lymph samples, and tissue samples. In embodiments, the isolated cells are obtained from a blood sample. In embodiments, the isolated cells are obtained from cells in culture. In embodiments, the isolated cells are peripheral blood mononuclear cells. In embodiments, the donor-sourced regulatory T cell population is separated from other T cells prior to expansion. While not wishing to be held by theory, Applicants have found that purifying regulatory T cells results in higher purity of results in enhanced expansion rate and quality of specificity. In embodiments, purifying regulatory T cells comprise selecting for or against CD4+ cells. Methods for sorting cells by cell surface markers are well known in the art. In embodiments, the separation is performed by fluorescence activated cell sorting (FACS).

According to the present invention, isolated cells are obtained from a biological sample. The isolated cells may be used directly in the culture step, or they may be further purified or concentrated prior to being cultured. For example, the sample may be blood which peripheral blood mononuclear cells (PBMC) are isolated. Methods of concentrating PBMC are known to those skilled in the art and include, density centrifugation (Ficoll-Paque), cell preparation tubes, and by SepMate tubes with freshly collected blood. The PBMC may be analyzed by a variety of methods, for example fluorescence-activated cell sorting (FACS), to determine the particular distribution of cell types within the sample. The PBMC may be further processed to isolate T cells, B cells, and/or dendritic cells (DC). Methods of concentrating cells are known to those skilled in the art and include, for example, flow cytometry and affinity column purification. The cells may be isolated and/or identified using molecules, such as antibodies, that bind to markers, thereby allowing the identification of particular target cells such as dendritic cells, B cells, T cells, and/or regulatory T cells (Treg cells). In embodiments, the identified Treg cells can then be separated and pooled, or otherwise concentrated, to increase the concentration of Treg cells in the sample. In embodiments, the concentration of Treg cells is increased by incubating the isolated cells with a molecule that binds Treg cells and then separating Treg cells from non-Treg cells by flow cytometry or magnetic cell separation. In embodiments, the molecules that bind Treg markers may be labeled with a detectable marker such as, for example, a florescent dye or a radiolabel. Suitable detectable markers are known to those skilled in the art.

B cells may be identified and/or isolated using the CD19 biomarker. For example, magnetic bead labeled CD19 antibodies are incubated with a sample comprising B cells. The magnetic bead labeled CD19 antibody will bind B cells and when a magnetic force is applied, the B cells which are bound to magnetic beads labeled CD19 antibody are separated from other components.

Dendritic cells may be identified and/or isolated using biomarkers. Plasmacytoid DCs (pDCs) and myeloid DCs (mDCs) can be isolated from human peripheral blood on the basis of the phenotypes CD303 (pDCs), CD1c⁺ (type 1 mDCs), and CD141 (type 2 mDCs). A pre-enrichment step may be performed on peripheral blood mononuclear cells (PBMCs), negatively selecting erythrocytes, platelets and peripheral leucocytes (that are not DCs), thereby providing a DC-enriched sample for enrichment and isolation.

Several biomarkers may be used to identify and/or isolate Treg cells. For example, all Treg cells express the CD4 and CD25 proteins, and thus are CD4+ and CD25+. Such proteins are referred to as markers, or marker proteins, for Treg cells. Thus, in one embodiment, the isolated cells comprise Treg cells that are at least CD4+CD25+. Such cells make up about 5-10% of the mature CD4+ T-cell population in humans, and about 1-2% of total lymphocytes. However, because the CD25 protein can also be expressed on non-regulatory cells during activation of the immune system, a more accurate identification of Treg cells in a cell population can be made by detecting expression of the transcription factor protein, forkhead box p3 (Foxp3). Thus, in embodiments, the isolated cells comprise Treg cells that are at least CD4+CD25+Foxp3+. A small percentage of Treg cells may express Foxp3, but express low to undetectable levels of CD25. Detection of the presence or absence of other marker proteins can improve this analysis even further. Such markers include, for example, Helios (a member of the Ikaros family of zinc finger proteins) and CD127. With regard to CD127, the absence or low levels of expression of this protein, as compared to intermediate (int) or high (hi) levels of expression, indicates the T-cell is a Treg. Methods of determining whether the expression level of CD127 is low, intermediate, or high, are disclosed herein and are known to those skilled in the art. In embodiments, the isolated cells comprise Treg cells that are CD4+CD25+CD127−/lo. In embodiments, the isolated cells comprise Treg cells that are CD4+CD25+CD127−/lo.

Provided herein are methods for expanding regulatory T cells. In embodiments, the T cells are antigen specific regulatory T cells. In embodiments, the antigen is an alloantigen. In embodiments, the antigen is autoantigen. The regulatory T cells may be cultured in the presence of a drug, molecule or agent capable of expanding the Treg population. Examples of expansion methods useful for practicing the present invention include, but are not limited to, contact with agonistic antibodies that link to receptors expressed on Treg cells (such as anti-CD3, anti-CD28, anti 4-1BB), cytokines (such as IL2, IL15), immunosuppressive drugs (such as rapamycin) and/or dendritic cells.

In an aspect is provided a method of producing a population of cells having antigen-specific regulatory T-cells (Treg cells), the method including isolating cells, including an initial population of regulatory T-cells, and culturing the isolated cells in the presence of one or more agents to expand at least of the initial, regulatory T-cells. In embodiments, the one or more agents are belatacept and an antigen-presenting cell. In embodiments, the one or more agents are an antigen-presenting cell. In embodiments, the isolated cells are cultured for one, two, three, or more days. In embodiments, the isolated cells are cultured in the presence of one or more agents in a first round and cultured in a second round in the presence of a different combination one or more agents. In embodiments the first round includes belatacept and an antigen-presenting cell. In embodiments, the second round includes an antigen-presenting cell. In embodiments, Treg cells (CD4+CD25+CD127−) are isolated after the second round and expanded with T cell activator.

Provided herein are methods of generating and expanding antigen-specific Treg from GvHD patients by anti-CD28 costimulatory blockade and APC stimulation. Specifically, methods provided herein are directed to collection and expansion of Treg cells from PBMC and exposure to antigens on the surface of the antigen presenting cells while costimulation through the CD28 receptor, thus producing Treg cells that recognize presented antigens. Such a population of Treg cells enable immunosuppression of activated T cells.

Provided herein are methods of forming a recipient antigen-specific regulatory T cell, the method including expanding a stem cell donor-sourced regulatory T cell in vitro, thereby forming an expanded regulatory T cell and contacting the expanded regulatory T cell with a recipient antigen presenting cell and a CD28 inhibitor compound in vitro; thereby forming an anergic recipient antigen-specific regulatory T cell.

In embodiments, the recipient antigen-specific regulatory T cell is formed by expanding a regulatory T cell from a donor in vitro, thereby forming an expanded regulatory T cells and contacting the expanded regulatory T cell with patient antigen presenting cell and a CD28 inhibitor compound in vitro; thereby forming the recipient antigen-specific regulatory T cells.

In embodiments, the regulatory T cell may be expanded from the subject or patient receiving the transplant or alternatively from a donor. In embodiments, the regulatory T cell may be isolated from a biological sample such as a blood sample.

In embodiments, the CD28 inhibitor may be a CD80/CD86 antagonist. In embodiments, the CD28 inhibitor is belatacept. Belatacept is a soluble fusion protein consisting of the modified extracellular domain of CTLA-4 fused to a portion (hinge-CH2-CH3 domains) of the Fc domain of a human immunoglobulin G1 antibody.

Provided herein are methods for generating recipient antigen-specific regulatory T cells comprising exposure to antigen presenting cells (APC). In embodiments, the antigen presenting cell may comprise a plurality of different recipient antigen presenting cells. In embodiments, the antigen presenting cell is a dendritic cell. In embodiments, the APC is derived from a recipient. In embodiments, the recipient suffers from GvHD.

### Compositions Including Antigen-Specific Regulatory T Cells

Provided herein are compositions including antigen-specific regulatory T cells derived from a donor of a tissue transplant. In embodiments, the Treg cells present one or more processed antigens from a subject who receives the tissue transplant. Thus, in an aspect is provided an antigen-specific regulatory T cell derived from a donor of a tissue transplant, wherein the antigen-specific regulatory T cell includes a T-cell receptor that specifically binds a transplant tissue antigen from a recipient of said transplant tissue. “Tissue antigen” is used in accordance with its common meaning and refers to an antigen that is expressed on a particular tissue or cell. In embodiments, the tissue antigen is an antigen expressed on a bone marrow transplant. In embodiments, the tissue antigen is an antigen expressed on a hematopoietic stem cell transplant.

In an aspect is provided a recipient antigen-specific regulatory T cell, wherein said recipient antigen-specific regulatory T cell is formed by methods provided herein including embodiments thereof.

The compositions provided herein include pharmaceutical compositions including the antigen-specific regulatory T cells provided herein including embodiments thereof. In an aspect is provided a pharmaceutical composition including a recipient antigen-specific regulatory T cell, wherein said recipient antigen-specific regulatory T cell is formed by a provided herein including embodiments thereof.

Provided herein are compositions comprising isolated regulatory T cells, wherein the regulatory T-cells have been expanded ex vivo and processed to become anergic. In embodiments, the composition is produced by culturing isolated cells comprising an initial population of regulatory T-cells in the presence of an agent to expand the initial regulatory T-cells. In embodiments, the isolated cells are produced by selecting for or against CD4+ cells. In embodiments, the regulatory T-cells are CD4+CD25+/highCD127−/low.

Pharmaceutical compositions provided by the present invention include compositions wherein the active ingredient (e.g. compounds provided herein, including embodiments or examples) may be contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. In embodiments, the dosage of a cell therapy can be estimated initially from preclinical data comparing the relative potency of a formulation with the standard formulation in animal studies. For example, a minimal cell dose to treat graft-versus host disease in animal studies can be compared between formulations.

It is understood that the examples and embodiments provided herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### P EMBODIMENTS

P Embodiment 1. A method of treating or preventing graft-versus-host disease in a subject in need thereof, the method comprising administering to the subject a therapeutically effective amount of recipient antigen-specific regulatory T cells; thereby treating or preventing graft-versus-host disease in the subject.

P Embodiment 2. The method of P embodiment 1, wherein the recipient antigen-specific regulatory T cells are formed by a method comprising: (a) expanding regulatory T cells in vitro, thereby forming a plurality of expanded regulatory T cells; and (b) contacting the plurality of expanded regulatory T cells with recipient antigen presenting cells derived from transplanted tissue and a CD28 inhibitor compound in vitro; thereby forming the recipient antigen-specific regulatory T cells.

P Embodiment 3. The method of P embodiment 2, wherein the transplanted tissue is transplanted bone marrow.

P Embodiment 4. The method of P embodiment 1, wherein the recipient antigen-specific regulatory T cells are formed by a method comprising: (a) expanding regulatory T cells in vitro, thereby forming a plurality of expanded regulatory T cells; (b) contacting recipient antigen presenting cells with recipient antigen in vitro to form activated recipient antigen presenting cells; and (c) contacting the plurality of expanded regulatory T cells with the activated recipient antigen presenting cells in the presence of CD28 inhibitor compound; thereby forming the recipient antigen-specific regulatory T cells.

P Embodiment 5. The method of P embodiment 2 or 4, wherein the recipient antigen presenting cells comprise a plurality of different recipient antigen presenting cells.

P Embodiment 6. The method of P embodiment 2 or 4, wherein the regulatory T cells are derived from the subject.

P Embodiment 7. The method of P embodiment 2 or 4, wherein the regulatory T cells are derived from a donor.

P Embodiment 8. The method of P embodiment 2 or 4, wherein the regulatory T cell is isolated from a biological sample of the subject or the donor.

P Embodiment 9. The method of P embodiment 8, wherein the biological sample is a blood sample.

P Embodiment 10. The method of any one of P embodiments 1-9, wherein the subject is a recipient of an allogeneic transplant.

P Embodiment 11. The method of any one of P embodiments 2-9, wherein the CD28 inhibitor compound is a CD80/CD86 antagonist.

P Embodiment 12. The method of any one of P embodiments 2-9, wherein the CD28 inhibitor compound is belatacept.

P Embodiment 13. The method of any one of P embodiments 2, 4 or 5, wherein the antigen presenting cell is a dendritic cell.

P Embodiment 14. A method of forming recipient antigen-specific regulatory T cells, the method comprising: (a) expanding regulatory T cells in vitro, thereby forming a plurality of expanded regulatory T cells; and (b) contacting the plurality of expanded regulatory T cells with activated recipient antigen presenting cells and a CD28 inhibitor compound in vitro; thereby forming recipient antigen-specific regulatory T cells.

P Embodiment 15. A method of forming recipient antigen-specific regulatory T cells, the method comprising: (a) expanding regulatory T cells in vitro, thereby forming a plurality of expanded regulatory T cells; (b) contacting recipient antigen presenting cells with a recipient antigen in vitro to form activated recipient antigen presenting cells; and (c) contacting the plurality of expanded regulatory T cells with the activated recipient antigen presenting cells; thereby forming the recipient antigen-specific regulatory T cells.

P Embodiment 16. The method of P embodiment 14 or 15, wherein the recipient antigen presenting cells comprise a plurality of different recipient antigen presenting cells.

P Embodiment 17. The method of P embodiment 14 or 15, comprising obtaining the regulatory T cells from a recipient or a donor of an allogeneic transplant.

P Embodiment 18. The method of P embodiment 17, wherein the regulatory T cells are isolated from a biological sample of the recipient or the donor of an allogeneic transplant.

P Embodiment 19. The method of P embodiment 18, wherein the biological sample is a blood sample.

P Embodiment 20. The method of any one of P embodiments 14-19, wherein the CD28 inhibitor compound is a CD80/CD86 antagonist.

P Embodiment 21. The method of any one of P embodiments 14-19, wherein the CD28 inhibitor compound is belatacept.

P Embodiment 22. The method of any one of P embodiments 14-16, wherein the antigen presenting cell is a dendritic cell.

P Embodiment 23. A pharmaceutical composition formed by the method of any one of P embodiments 14-22, wherein the recipient antigen-specific regulatory T cells are an anergic recipient antigen specific regulatory T cells.

### EMBODIMENTS

Embodiment 1. A method of treating or preventing graft-versus-host disease in a subject in need thereof, the method comprising administering to the subject a therapeutically effective amount of recipient antigen-specific regulatory T cells, thereby treating or preventing graft-versus-host disease in said subject; wherein said recipient antigen-specific regulatory T cells are derived from regulatory T cells from a donor of a tissue transplant to said subject; and wherein said tissue transplant is a cause of graft-versus-host disease in said subject.

Embodiment 2. The method of embodiment 1, wherein said regulatory T cells are obtained from said donor within one week said tissue transplant was obtained from said donor.

Embodiment 3. The method of embodiment 1 or 2, wherein said regulatory T cells are isolated from said donor's blood.

Embodiment 4. The method of any one of embodiments 1-3, wherein said tissue transplant comprises hematopoietic stem cells.

Embodiment 5. The method of any one of embodiments 1-4, wherein said subject has or previously had cancer.

Embodiment 6. The method of embodiment 5, wherein said cancer is leukemia, lymphoma, sarcoma, myeloma, or glioma.

Embodiment 7. The method of any one of embodiments 1-6, wherein said recipient antigen-specific regulatory T cells are formed by a method comprising: (a) expanding said regulatory T cells in vitro, thereby forming a plurality of regulatory T cells; and (b) contacting said plurality of regulatory T cells with a plurality of recipient antigen presenting cells and a CD28 inhibitor compound in vitro, thereby forming said recipient antigen-specific regulatory T cells.

Embodiment 8. The method of embodiment 7, wherein step (b) further comprises contacting said plurality of regulatory T cells with a second plurality of recipient antigen presenting cells.

Embodiment 9. The method of embodiment 7 or 8, wherein said plurality of recipient antigen presenting cells are taken from said subject's blood.

Embodiment 10. The method of any one of embodiments 7-9, wherein said plurality of recipient antigen presenting cells are taken from said subject's tissue transplant.

Embodiment 11. The method of any one of embodiments 7-10, wherein said plurality of recipient antigen presenting cells are taken from said subject when the subject has graft-versus-host disease.

Embodiment 12. The method of any one of embodiments 7-11, wherein said plurality of recipient antigen presenting cells are taken from said subject prior to administration of a graft-versus-host disease therapeutic treatment.

Embodiment 13. The method of embodiment 12, wherein said graft-versus-host disease therapeutic treatment comprises a corticosteroid and/or immunosuppressive compound.

Embodiment 14. The method of embodiment 13, wherein said immunosuppressive compound is Methotrexate, Cyclosporine, Tacrolimus, Mycophenolate mofetil, Sirolimus, Antithymocyte globulin, Alemtuzumab, Cyclophosphamide or Inolimomab.

Embodiment 15. The method of embodiment 14, wherein said corticosteroid is prednisone, methylprednisolone, dexamethasone, beclomethasone or budesonide.

Embodiment 16. The method of any one of embodiments 7-15, wherein said plurality of recipient antigen presenting cells comprise dendritic cells.

Embodiment 17. The method of any one of embodiments 7-16, wherein said plurality of recipient antigen presenting cells comprise B cells.

Embodiment 18. The method of any one of embodiments 7-17, wherein said CD28 inhibitor compound is a CD80/CD86 antagonist.

Embodiment 19. The method of embodiment 18, wherein the CD28 inhibitor compound is abatacept or belatacept.

Embodiment 20. The method any one of embodiments 1-4, wherein said recipient antigen-specific regulatory T cells are formed by a method comprising: (a) expanding regulatory T cells in vitro, thereby forming a plurality of regulatory T cells; (b) contacting a plurality of recipient antigen presenting cells with recipient antigen in vitro, thereby forming a plurality of activated recipient antigen presenting cells; and (c) contacting said plurality of regulatory T cells with said plurality of activated recipient antigen presenting cells in the presence of a CD28 inhibitor compound, thereby forming said recipient antigen-specific regulatory T cells.

Embodiment 21. The method of embodiment 20, wherein step (c) further comprises contacting said plurality of regulatory T cells with a second plurality of activated recipient antigen presenting cells.

Embodiment 22. The method of embodiment 20 or 21, wherein said plurality of recipient antigen presenting cells are taken from said subject's blood.

Embodiment 23. The method of any one of embodiments 20-22, wherein said plurality of recipient antigen presenting cells are taken from said subject's tissue transplant.

Embodiment 24. The method of any one of embodiments 20-23, wherein said plurality of recipient antigen presenting cells are taken from said subject when the subject has graft-versus-host disease.

Embodiment 25. The method of any one of embodiments 20-24, wherein said plurality of recipient antigen presenting cells are taken from said subject prior to administration of a graft-versus-host disease therapeutic treatment.

Embodiment 26. The method of embodiment 25, wherein said graft-versus-host disease therapeutic treatment comprises a corticosteroid and/or immunosuppressive compound.

Embodiment 27. The method of embodiment 26, wherein said immunosuppressive compound is Methotrexate, Cyclosporine, Tacrolimus, Mycophenolate mofetil, Sirolimus, Antithymocyte globulin, Alemtuzumab, Cyclophosphamide or Inolimomab.

Embodiment 28. The method of embodiment 26, wherein said corticosteroid is prednisone, methylprednisolone, dexamethasone, beclomethasone or budesonide.

Embodiment 29. The method of any one of embodiments 20-28, wherein said plurality of recipient antigen presenting cells comprise dendritic cells.

Embodiment 30. The method of any one of embodiments 20-29, wherein said plurality of recipient antigen presenting cells comprise B cells.

Embodiment 31. The method of any one of embodiments 20-30, wherein the CD28 inhibitor compound is a CD80/CD86 antagonist.

Embodiment 32. The method of embodiment 31, wherein the CD28 inhibitor compound is abatacept or belatacept.

Embodiment 33. A method of forming recipient antigen-specific regulatory T cells, the method comprising: (a) expanding regulatory T cells in vitro, wherein said regulatory T cells are from a donor of a tissue transplant, thereby forming a plurality of regulatory T cells; and (b) contacting said plurality of regulatory T cells with a plurality of recipient antigen presenting cells and a CD28 inhibitor compound in vitro, wherein said plurality of recipient antigen presenting cells is from a subject who has received said tissue transplant, thereby forming said recipient antigen-specific regulatory T cells.

Embodiment 34. The method of embodiment 33, wherein step (b) further comprises contacting said plurality of regulatory T cells with a second plurality of recipient antigen presenting cells.

Embodiment 35. The method of embodiment 33 or 34, wherein said plurality of recipient antigen presenting cells is taken from said subject's blood.

Embodiment 36. The method of any one of embodiments 33-35, wherein said plurality of recipient antigen presenting cells is taken from said subject's tissue transplant.

Embodiment 37. The method of any one of embodiments 33-36, wherein said plurality of recipient antigen presenting cells is taken from said subject when the subject has graft-versus-host disease.

Embodiment 38. The method of any one of embodiments 33-37, wherein said plurality of recipient antigen presenting cells is taken from said subject prior to administration of a graft-versus-host disease therapeutic treatment.

Embodiment 39. The method of embodiment 38, wherein said graft-versus-host disease therapeutic treatment comprises a corticosteroid and/or immunosuppressive compound.

Embodiment 40. The method of embodiment 39, wherein said immunosuppressive compound is Methotrexate, Cyclosporine, Tacrolimus, Mycophenolate mofetil, Sirolimus, Antithymocyte globulin, Alemtuzumab, Cyclophosphamide or Inolimomab.

Embodiment 41. The method of embodiment 39, wherein said corticosteroid is prednisone, methylprednisolone, dexamethasone, beclomethasone or budesonide.

Embodiment 42. The method of any one of embodiments 33-41, wherein said plurality of recipient antigen presenting cells comprise dendritic cells.

Embodiment 43. The method of any one of embodiments 33-42, wherein said plurality of recipient antigen presenting cells comprise B cells.

Embodiment 44. The method of any one of embodiments 33-43, wherein said CD28 inhibitor compound is a CD80/CD86 antagonist.

Embodiment 45. The method of embodiment 44, wherein the CD28 inhibitor compound is abatacept or belatacept.

Embodiment 46. A method of forming recipient antigen-specific regulatory T cells, the method comprising: (a) expanding regulatory T cells in vitro, wherein said regulatory T cells are from a donor of a tissue transplant, thereby forming a plurality of regulatory T cells; (b) contacting a plurality of recipient antigen presenting cells with a recipient antigen in vitro, wherein said plurality of recipient antigen presenting cells is from a subject who has received said tissue transplant, thereby forming a plurality of activated recipient antigen presenting cells; and (c) contacting said plurality of regulatory T cells with said plurality of activated recipient antigen presenting cells and a CD28 inhibitor compound in vitro, thereby forming said recipient antigen-specific regulatory T cells.

Embodiment 47. The method of embodiment 46, wherein step (c) further comprises contacting said plurality of regulatory T cells with a second plurality of activated recipient antigen presenting cells.

Embodiment 48. The method of embodiment 46 or 47, wherein said plurality of recipient antigen presenting cells are taken from said subject's blood.

Embodiment 49. The method of any one of embodiments 46-48, wherein said recipient antigen presenting cells are taken from said subject's tissue transplant.

Embodiment 50. The method of any one of embodiment 46-49, wherein said recipient antigen presenting cells are taken from said subject prior to administration of a graft-versus-host disease therapeutic treatment.

Embodiment 51. The method of embodiment 50, wherein said graft-versus-host disease therapeutic treatment comprises a corticosteroid and/or immunosuppressive compound.

Embodiment 52. The method of embodiment 51, wherein said immunosuppressive compound is Methotrexate, Cyclosporine, Tacrolimus, Mycophenolate mofetil, Sirolimus, Antithymocyte globulin, Alemtuzumab, Cyclophosphamide or Inolimomab.

Embodiment 53. The method of embodiment 51, wherein said corticosteroid is prednisone, methylprednisolone, dexamethasone, beclomethasone or budesonide.

Embodiment 54. The method of any one of embodiments 46-53, wherein said plurality of recipient antigen presenting cells comprise dendritic cells.

Embodiment 55. The method of any one of embodiments 46-54, wherein said plurality of recipient antigen presenting cells comprise B cells.

Embodiment 56. The method of any one of embodiments 46-55, wherein said CD28 inhibitor compound is a CD80/CD86 antagonist.

Embodiment 57. The method of embodiment 56, wherein the CD28 inhibitor compound is abatacept or belatacept.

Embodiment 58. An antigen-specific regulatory T cell derived from a donor of a transplant tissue, wherein said antigen-specific regulatory T cell specifically comprises a T-cell receptor that specifically binds a transplant tissue antigen from a recipient of said transplant tissue.

Embodiment 59. A pharmaceutical composition comprising recipient antigen-specific regulatory T cells, wherein said recipient antigen-specific regulatory T cells are formed by the method of any one of embodiments 33-57.

### EXAMPLES

### Example 1: Characterization of PBMCs

Isolation of PBMC from Donor or Patient's Leukopak by Ficoll Density Gradient Separation Protocol

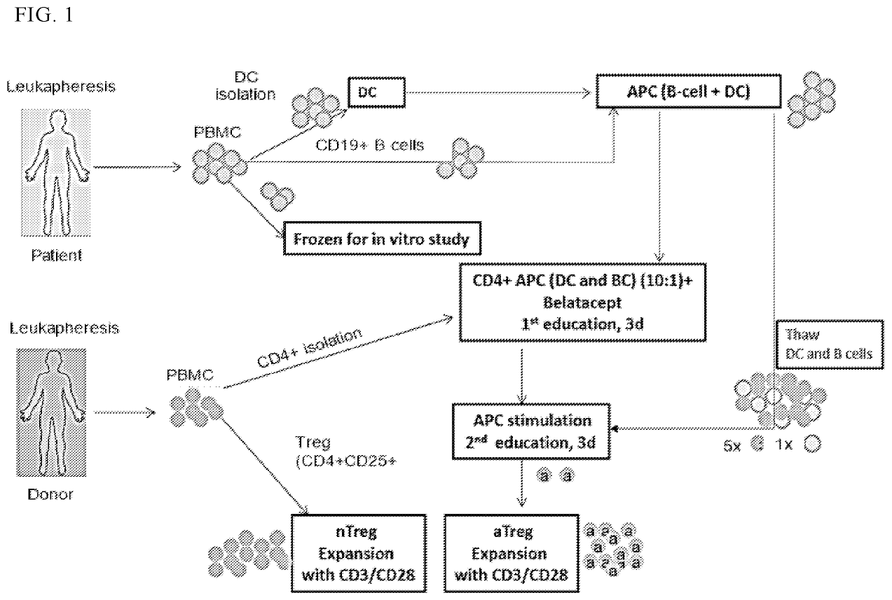

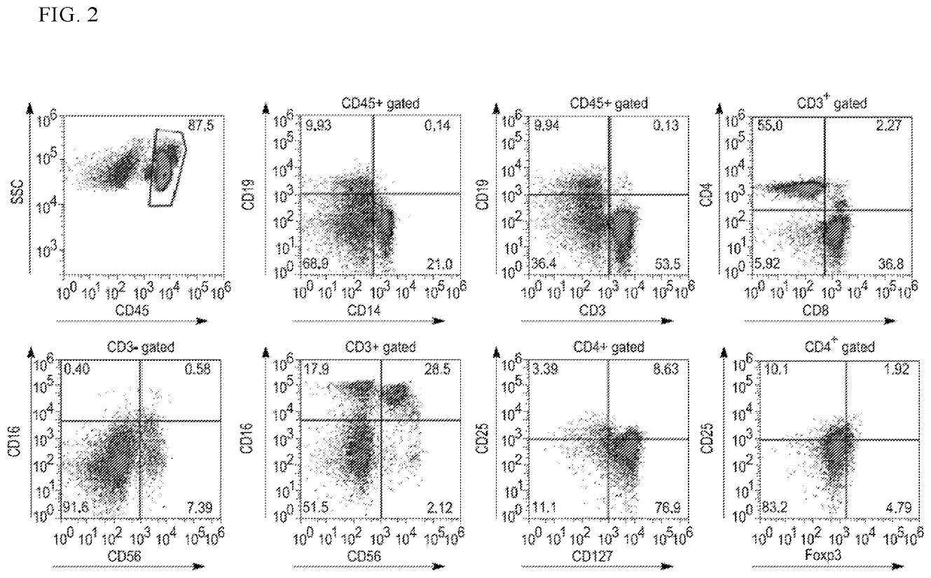

See flow chart in FIG. 1.

PBMCs were collected via leukophresis procedures. Cells from the leukopak were then transferred into sterile bottles. The cells were diluted by PBS-HSA buffer. Subsequently, 25 ml of diluted blood suspension was carefully layered over 15 ml of Ficoll-Paque in a 50 ml conical tube.

The conical tube was centrifuged at 400 g (Acc5, Dec0) for 30 min at 20° C. The light yellow upper layer was removed without disturbing white membrane layer at the interphase. The white membrane layer was transferred from original tube to new 50 ml conical tube. PBS-HSA buffer was added to the tube (50 ml final), mixed, and centrifuged at 1200 rpm for 10 minutes at 20° C. The supernatant was removed.

Cell pellets were resuspended in autoMACS running buffer and transferred into a 500 ml bottle. The empty tubes that had the pellet were washed and the rinse was added into the same bottle. The volume in the bottle was measured and mixed well. The cells were then counted.

Flow Cytometric Analysis for Surface Marker of PBMCs

Cells were pelleted in pre-labeled tubes. Next, 50 ul of antibody master mix shown in Table 1 was added into the tube and an unstained sample was prepared as a control. Tubes were incubated in dark for 20 min at room temperature. Then, 200 ul of FACS staining buffer was added and tubes were spun down at 8000 rpm for 2 min. The supernatant was carefully removed and 200 ul of FACS staining buffer was added to resuspend the pellet. The samples were analyzed by using flow cytometry within 2 hrs.

Flow Cytometric Analysis for Foxp3 Expression

The cells were pelleted in pre-labeled tubes. Then, 50 ul of the surface antibody master mix shown in Table 1 was added into the tube and unstained sample was prepared as control. Tubes were incubated in dark for 20 min at room temperature. Subsequently, 200 ul of FACS staining buffer was added and the tubes spin down at 8000 rpm for 2 min.

The supernatant was removed carefully and 200 ul of 1× Transcription Factor Fix solution was added to each tube and mixed by gently pipetting up and down. Tubes were incubated in the dark at room temperature for 60 min. Tubes were centrifuged at 8,000 rpm for 2 min and the supernatant was discarded.

Then, 200 ul of 1× Perm buffer was added to each tube and mixed by gently pipetting up and down. Tubes were centrifuged at 8,000 rpm for 2 min. Supernatant was discarded. The wash was repeated for a total of two washes.

A volume of 200 ul of 1× Perm buffer was added to each tube and mixed by gently pipetting up and down. Tubes were incubated in dark for 30 min. The tubes were centrifuged at 8,000 rpm for 2 min and the supernatant was discarded.

After, 50 ul of FOXP3-BV421 antibody dilution was added to each tube, except unstained control and mixed by gently pipetting up and down. Tubes were incubated in dark for 60 min.

300 ul of 1× Perm buffer was added and mixed by gently pipetting up and down. The tubes were centrifuged at 8,000 rpm for 2 min, and the supernatant was discarded. This was repeated two times for a total of three washes.

Cells were resuspended in 300 ul of FACS buffer and transferred to FACS tubes. Data were acquired and analyzed using a SA3800 Sony Spectral Analyzer.

TABLE 1
FACS Antibody staining panel of PBMC

| PBMC panel | APC panel | Foxp3 panel |
|---|---|---|
| CD16-BV421 | CD1c-PE | Foxp3-BV421 |
| CD8-Pacific blue | CD303-FITC | CD8-Pacific blue |
| CD4-AF488 | CD141-APC | CD4-AF488 |
| CD56-PE | | CD127-PE |
| CD19-BV605 | | CD19-BV605 |
| | | CD25-APC |
| CD14-Percp-Cy5.5 | | CD14-Percp-Cy5.5 |
| CD45-AF700 | | CD45-AF700 |
| CD3-PE-Cy7 | | CD3-PE-Cy7 |

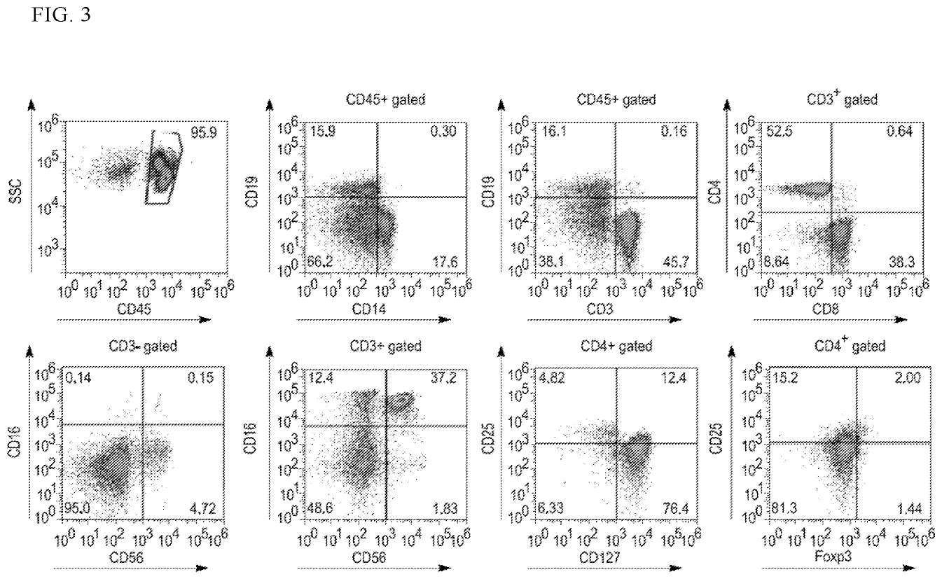

FIG. 2 and FIG. 3 show phenotypic analysis results of FACS. The results indicated that PBMCs from both donor and patient had normal phenotype, which contained 10-20% of CD19+ B cells, 10-20% of CD14+ monocytes and 40-50% of CD3+ T cells.

### Example 2: Generation and Expantion of Antigen-Specific T Regulatory Cells from Graft Versus Host Disease Patients by Belatacept Treatment and Antigen Presenting Cell Stimulation

In order to generate and expand a population of antigen-specific regulatory T cells, the experimental protocol as outlined in FIG. 1 was followed. Dendritic cells (DC) and CD19+ B cells were isolated from the PBMCs, and a portion of the PBMCs were frozen for in vivo studies.

The isolated DC and B cells were combined to create an APC cell population to be frozen and used in the education of Treg cells isolated from a donor. PBMCs isolated from the donor via leukapheresis were segregated into a CD4+ population and a Treg population (CD4+CD25+CD127−). This latter population was expanded with exposure to CD3/CD28. The isolated CD4+ cells were combined with APC from patient plus belatacept in a 1st education 3 day step. This APC population was stimulated in a 2nd education 3 day step after combination with the frozen APC sample. After the 2nd education, Treg cells (CD4+CD25+CD127−) were sorted and expanded with CD3/CD28 exposure.

CD19+ B Cell Isolation Protocol

CD19+ cells were isolated with CD19 MicroBeads (Miltenyi, Cat. no. 130-050-301) using auto-MACS-pro and according to company's protocol.

CD19+ cells first were labeled by CD19 microbeads. AutoMACS running buffer (Contains BSA 0.5%, EDTA, PBS, 0.09% Azide, PH7.2) was pre-cooled to 4° C. PBMC cell pellets (50 E7) were resuspended with 10 ml pre-cooled autoMACS running buffer (the cell pellets were resuspended in 80 ul of auto MACS running buffer per 1E7 total cells. 50×80 ul=4 ml). Then, 1 ml of CD19+ MicroBeads were added (20 ul of CD19+ MicroBeads were added per 1E7 total cells. 50×20 ul=1000 ul).

The resuspended cells were mixed well and incubated for 15 min in a refrigerator (2-8° C.). The cell suspension was transferred to a 250 ml tube. Subsequently, 50 ml autoMACS running buffer was added to the tube to wash cells (1-2 ml of buffer was added per 1E7 cells 50 ml).

The tubes were centrifuged at 1200 rpm for 10 minutes at 4° C. The supernatant was removed. The cell pellet was resuspended with auto MACS running buffer. The total volume was 2.5 ml in one 15 ml conical tube (50E7 cells resuspended in 2.5 ml of auto MACS running buffer. The cell concentration was 2E8 cells/ml). It is noted that 1E8 cells were resuspended in a minimum of 500 ul of auto MACS running buffer. Cell concentration was less than or equal to 2E8 cells/ml.

Magnetic Bead Labeled CD19+ B Cells Separation with the autoMACS® Pro Separator Protocol

For setting up the autoMACS® pro separator system, the autoMACS running buffer was balanced at room temperature. The instrument was prepared and primed as follows:

machine was turned on, buffer level was checked, and the machine system was rinsed). The tube containing the sample was applied, and two 15 ml conical tubes were provided and labeled for collecting the unlabeled and labeled cell fractions.

Sample tubes were placed in row A of the tube rack and the fraction collection tubes were placed in rows B and C. The program “Positive selection: Possel” was chosen for standard separation.

The positive fraction was collected in row C of the tube rack and the cell count was performed.

Blood Dendritic Cell Isolation Protocol

Human blood dendritic cells from patient were isolated with a kit (Miltenyi, Cat. no. 130-091-379) using auto-MACS-pro.

Magnetic Bead Labeling of Non-Dendritic Cells

The buffer used was pre-cooled MACS running buffer. Freshly isolated PBMC cells were pelleted by centrifugation at 1200 rpm for 10 min and resuspended in 300 ul of pre-cooled buffer (prepared as described above) per 1E8 total cells for each PBMCs sample. This may be scaled up to a maximum of 6E9 cells.

After, 100 μL of FcR Blocking Reagent and 100 μL of Non-DC Depletion Cocktail were added per 10⁸ total cells. Samples were mixed and then incubated for 15 minutes at 2-8° C. Cells were washed by adding 5-10 mL of buffer per 10⁸ cells and centrifuging at 300×g for 10 minutes. The supernatant was removed.

The cell pellet was resuspended in buffer using 500 μL for up to 1×10⁸ cells.

Cell Separation with the autoMACS® Pro Separator: Depletion of Non-Dendritic Cells

The instrument was first prepared and primed. The tube containing the sample was applied and tubes for collecting the labeled and unlabeled cell fractions were provided. The sample tube was placed in row A of the tube rack and the fraction collection tubes in rows B and C. For depletion, the program “depletes” was selected. Negative fractions were collected in row B of the tube rack.

Magnetic Bead Labeling of Pre-Enriched Dendritic Cells

The cell suspension was centrifuged and supernatant removed. The cell pellet was then resuspended in 400 uL of buffer/1E8 cells. Afterwards, 100 uL of DC enrichment cocktail was added for every 1E8 cells. Suspensions were mixed and then incubated for 15 min at 2-8° C.

The cells were washed by adding 5-10 mL of buffer and centrifuged at 300×g for 10 min. The supernatant was removed. Then, 1E8 cells were resuspended in 500 uL of buffer.

Cell Separation with the autoMACS® Pro Separator: Positive Selection of Dendritic Cells

First, the instrument was prepared and primed. The tube containing the sample was applied and tubes were provided for collecting the labeled and unlabeled cell fractions. The sample tube was placed in row A of the tube rack and the fraction collection tubes were placed in rows B and C. The program used was depletion: “Posseld2”. The negative fraction was collected in row C of the tube rack.

The cell number was counted and 0.5×10⁶ cells were taken out for FACS analysis.

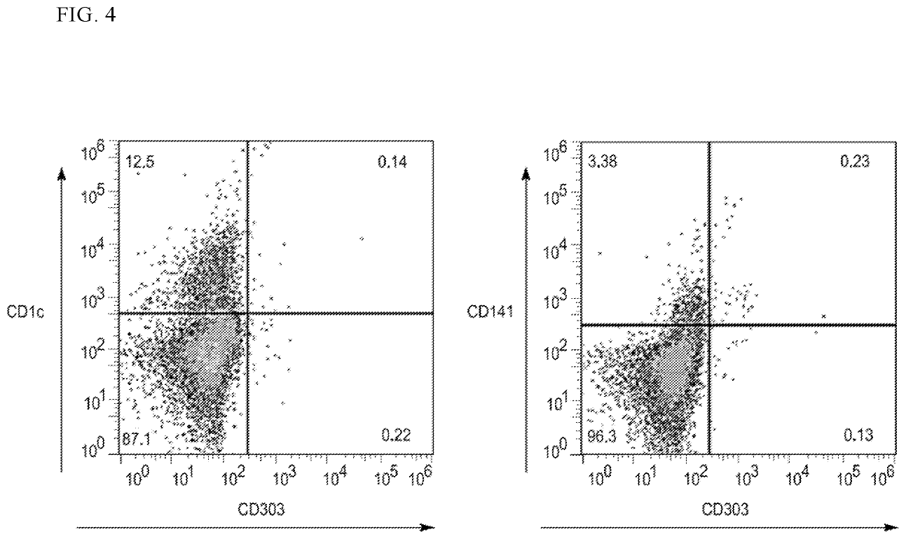

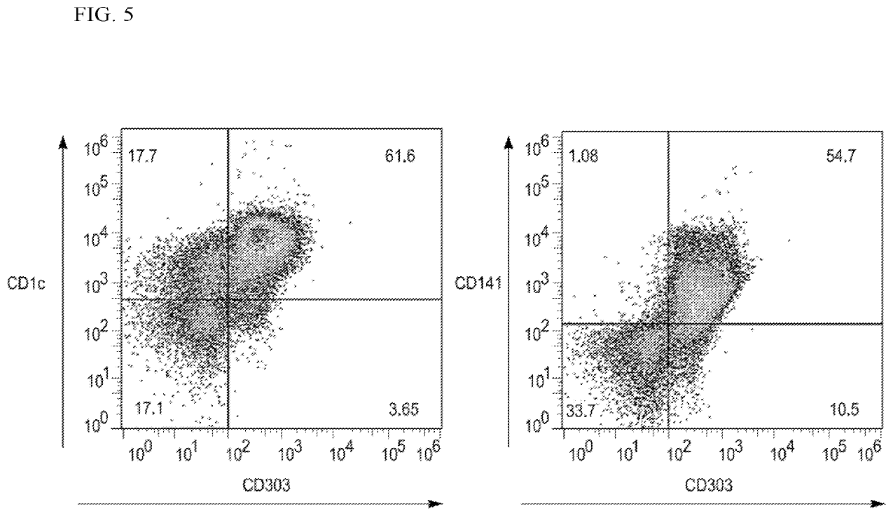

FIG. 4 and FIG. 5 show results of phenotypic analysis of PBMC for CD1c+ or CD303+ and CD141+ from a patient before selection and after the selection process. The results illustrate that PBMCs express very low levels of CD1c, CD303 and CD141 before DCs enrichment, but 60-70% of the population were DCs after enrichment.

CD4+ T Cell Isolation Protocol

Donor CD4+ T cells were isolated with a kit (Miltenyi, Cat. no. 130-096-533) using auto-MACS-pro.

Magnetic Bead Labeling of Non-CD4+ Cells

First, 10 ul of CD4+ T Cell Biotin-Antibody cocktail per 1E7 total cells was added for PBMCs sample. Suspensions were mixed and then incubated for 15 min at 2-8° C. Auto MACS running buffer at a volume of 30 ul per 1E7 cells was added for PBMCs sample. Then, 20 ul of CD4+ T Cell Microbead cocktail per 1E7 cells was added for PBMCs sample.

Suspensions were mixed and then incubated for 10 min at 2-8° C. The recommended final cell concentration was 1E7 cells/100 ul. Details for PBMCs magnetic labeling are shown in the table below.

It is noted that if the cell number was too low, the final volume was adjusted to a minimum of 500 ul of auto MACS running buffer.

Magnetic Bead Labeled CD4+ T Cells Separation with the autoMACS® Pro Separator

For setting up the autoMACS® Pro Separator system, the autoMACS running buffer was balanced at room temperature. The instrument was prepared and primed as follows: the machine was turned on, buffer levels were checked, and the machine system was rinsed.

The tube containing the sample was applied, and two 15 ml conical tubes were labeled for collecting the unlabeled and labeled cell fractions. The sample tube was placed in row A of the tube rack and the fraction collection tubes were placed in rows B and C.

For a standard separation the following program was selected: Depletion: Depletes. The negative fraction in row B of the tube rack was collected and the cell number was counted.

First Education Protocol

CD4+ T cell (donor) were cultured with APCs (DCs and B cell) from patient in the presence of belatacept for the Pt education.

B cells and DCs were mixed at 1:1 ratio. For each, 10E6 cells were used. The cell suspension mixture was centrifuged at 1200 rpm for 10 min at 20° C. The supernatant was removed. The cells were resuspended in 10 ml X-vivo 20 complete medium.

Culture CD4+ T Cell with APCs (DCs and B Cell) in the Presence of Belatacept (Ratio of CD4+ Tcells:APCs was 10:1)

The final cell concentration of the culture system was 1E6 cells/ml. The total number of cells was 120 E6. Accordingly, 120 ml of X-vivo 20 complete medium with IL-2 and belatacept was required.

First, the DCs and B cell mixture suspension was mixed with the CD4+ T cell suspension, then added to aT175 flask. The original conical tubes were washed with X-vivo 20 complete medium with IL-2 (the final concentration was 10 IU/ml) and belatacept (the final concentration was 40 ug/ml) and added to the T175. Any remaining X-vivo 20 complete medium with IL-2 and belatacept media was added to the flask. Final volume in flask was 120 ml.

The T175 flask was placed in an incubator at 5% CO₂ and 37° C. for 3 days.

CD25+ T Cell Isolation Protocol

Donor CD25+ T Cells were isolated with isolation Kit (Miltenyi, Cat. no. 130-032-501/325-01) using auto-MACS-pro.

Cell pellets were suspended in 90 ul of auto MACS running buffer per 1E7 total cells for each PBMCs sample. Then, 10 ul of CD25 Microbead per 1E7 total cells was added for each PBMCs sample. Suspensions were mixed and then incubated for 15 min at 2-8° C. Cells were washed by adding 1 ml buffer per 1E7 total cells and centrifuged at 1200 rpm for 10 min.

The supernatant was removed, and the pellets were resuspended with running buffer for a concentration of 1E8 cells/ml.

Magnetic Bead Labeled CD25+ T Cells Separation with the autoMACS® Pro Separator

The tube containing the sample was applied. Two 15 mL conical tubes were provided and labeled for collecting the labeled and unlabeled cell fractions, respectively. The sample tube was placed in row A of the tube rack and the fraction collection tubes were placed in rows B and C (for each PBMC sample).

For a standard separation, the program Dep105 was selected. The negative fraction in row B of the tube rack for each PBMCs sample was collected. The cell count was performed.

Culture Protocol: Selected CD25+ T Cells for Non Antigen Specific Treg Cells Sorting

The selected CD25+ T cell suspension was spun down at 1200 rpm for 10 min at 20° C., and the supernatant was removed. The pellet was loosened and resuspended with 10 ml x-vivo 20 complete medium. The cell suspension was transferred into a T25 flask. The conical tube was washed once with 10 ml X-vivo 20 complete medium. The wash suspension was transferred into same flask. The cell concentration was 2E6 cells/mL.

The flask was incubated at 5% CO₂ and 37° C. in an incubator overnight for Treg cells sorting on Day 1.

### Example 3: Sorting and Expanding nTreg Cells

Protocol for Sorting nTreg Cells

The overnight-cultured CD25+T cells suspension was collected in a 50 ml tube. The cells were stained with CD4-AF488, CD25-APC, CD127-PE antibodies. The cell concentration was 2E7 cells/ml and the antibodies concentration was 1 ul/1E6 cells.

The cells were incubated in the dark, for 20 min, at room temperature. After incubation, 40 ml HBSS-HSA buffer was added into tube and centrifuged at 1200 rpm for 10 minutes at 20° C. The supernatant was removed and cells were resuspended with 3.5 ml of HBSS-HSA buffer for sorting.

To collect CD4+CD25+CD127−/lo Treg into 15 ml tubes after sorting, 2 collection tubes were prepared with 5 ml X-vivo20 complete medium.

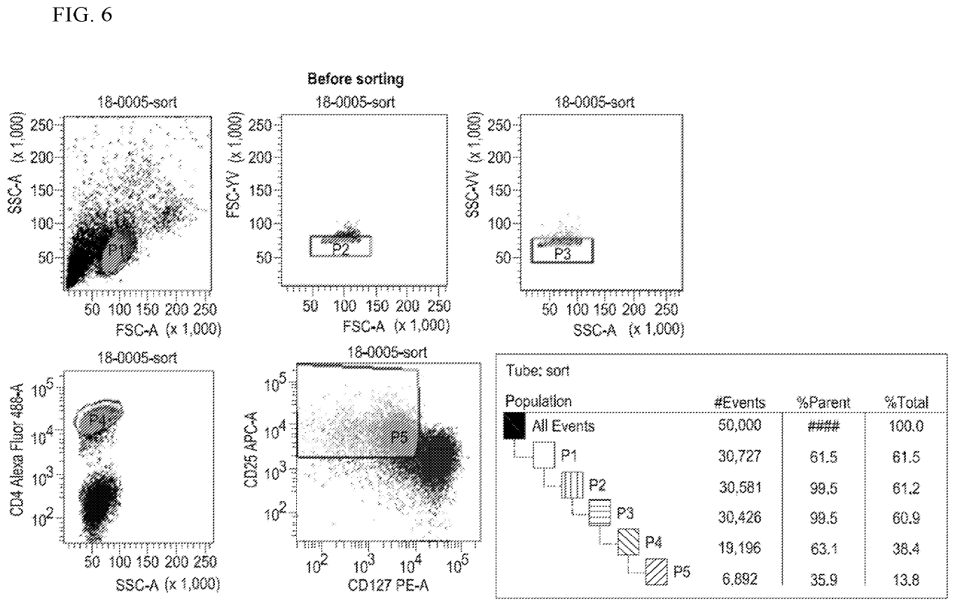

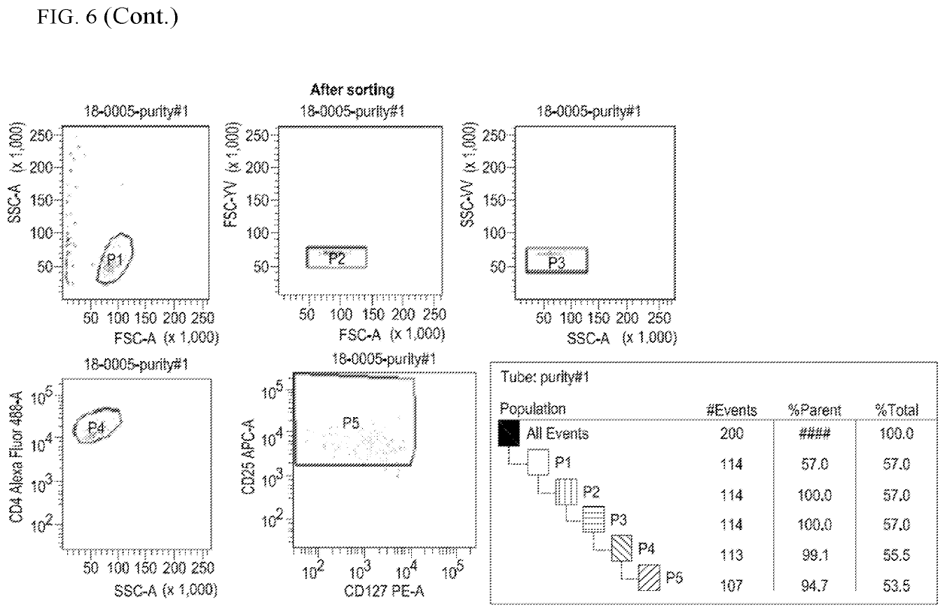

FIG. 6 show marker and cytokine expression analysis of nTreg isolation. The results illustrate that the purity of sorted Treg was 94.7% and were of high quality for expansion.

nTreg Stimulation Protocol

CD4+CD25+CD127−/lo Treg cells were stimulated using CD3/CD28/CD2 T cell activators

### Example 4: Preparation of CD19+ B Cells and Dendritic Cells for Second Education Usage

Two vials of CD19+ B cells and 2 vials DC cells from liquid nitrogen tank were thawed using warm X-vivo20 complete medium. The cells were spun down at 1200 rpm for 10 minutes at 20° C. The supernatant was carefully and completely removed. The cell pellet was resuspended with 5 ml X-vivo 20 complete medium. The cells were then counted.

The cell suspension was transferred into T25, and x-vivo 20 complete medium was used to wash the tube once. The wash suspension was transferred into the flask. The flask was incubated at 5% CO2 and 37° C. in an incubator overnight. The culture flask remained upright.

For nTreg: IL-2 was added into nTreg culture plate. The final concentration of IL-2 was 500 U/ml.

Phenotype Analysis of First Education Product: CD4+ T Cell and Culturing

The cell suspension was collected into a 250 ml tube. The flasks were washed with 10 ml PBS-HSA buffer, and the wash was transferred into the same 250 ml tube. The flask was washed with HBSS-HSA buffer again, and the wash suspension was transferred into the same 250 ml tube. The cells were spun down at 1200 rpm for 10 min and the supernatant was removed.

The below FACS panel was used for Foxp3 staining: unstained control and stained sample were tested.

Foxp3 panel
Foxp3-BV421
CD8-Pacific blue
CD4-AF488
CD127-PE
CD19-BV605
CD25-APC
CD14-Percp-Cy5.5
CD3-PE-Cy7

The pellet was resuspended. The cell number of cultured CD4+ T cells from the 1^{st} education was counted and 0.5 million were taken out for FACS analysis. The remaining cells were used for the 2^{nd} education.

Collect B Cells and DCs

The cell suspension was mixed well and transferred to a 50 ml tube. The flask was washed and the wash was transferred to the tube. The solution was mixed well and cells were counted.

The same amount of B cells and DC cells were mixed into the same 50 ml tube. The cells were spun down and supernatant was removed. The cells were resuspended in 10 ml of X-vivo 20 complete medium.

### Example 5: Second Education and Characterization

Co-Culture First-Educated CD4+T Cell with DCs for 2^{nd} Education

First, 10 ml DCs and B cells were transferred into T175 flask containing the educated CD4+ T cells for second education. The ratio of CD4+ to APCs was 5:1. The final concentration of cells was 1E6 cells/ml and IL-2 concentration was 10 U/ml.

The cells were incubated at 5% CO₂ at 37° C. in an incubator for 3 days.

Sort aTreg Cells

For nTreg: cells were transferred into T25 and the culture volume was tripled with complete media and IL-2 (500 IU/ml) into T25. Fresh media was added into culture every 2-3 days.

The CD4+ T cell suspension was collected into 250 ml tube. The cells were stained with CD4-AF488, CD25-APC, CD127-PE antibodies. The cell concentration was 2E7 cells/nil and the antibodies concentration was 1 ul/1E6 cells.

The cells were incubated in the dark for 20 min, at room temperature. After incubation, 40 ml of HBSS-HSA buffer was added into a tube, and the tube was centrifuged at 1200 rpm for 10 min at 20° C. The supernatant was removed and the cells were resuspended with 7 ml of HBSS-HSA buffer for sorting.

CD4+CD25+CD127−/lo Treg were collected into 15 ml tubes after sorting and 2 collection tubes were prepared with 5 ml X-vivo20 complete medium.

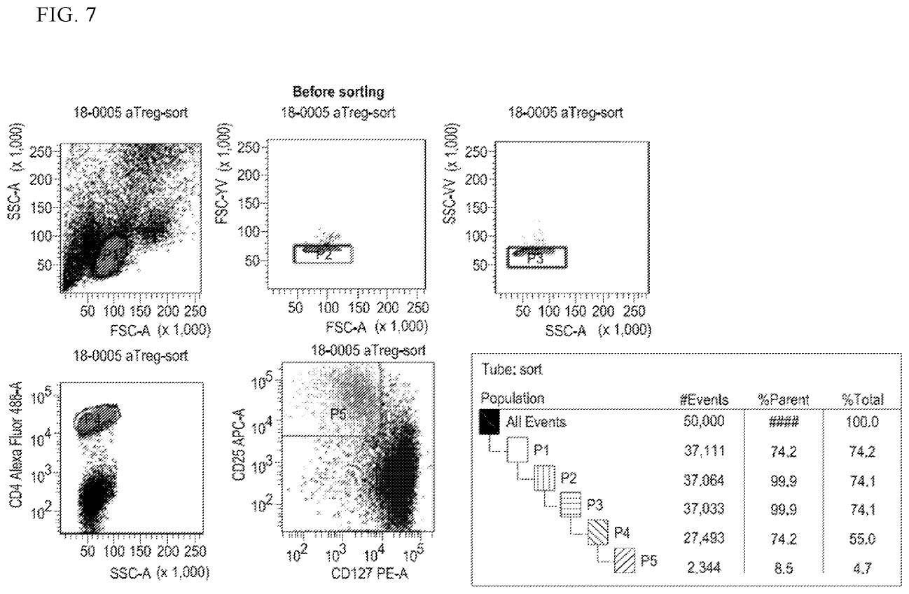

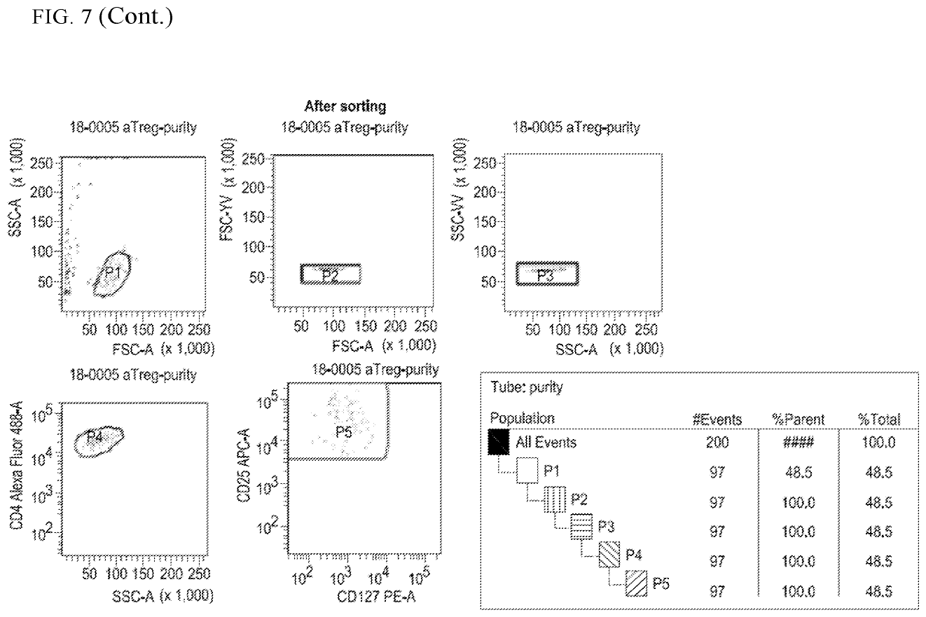

Results are shown in FIG. 7, and illustrate that the purity of sorted aTreg cells was 100% and were good quality for expansion.

FACS Staining for Foxp3

First, 2E6 CD4+ cells were taken out of the 2^{nd} education cell suspension for FACS staining. The FACS panel for unstained control and stained sample were as follows:

Foxp3 panel
Foxp3-BV421(1:25)
CD8-Pacific blue
CD4-AF488
CD127-PE
CD19-BV605
CD25-APC
CD14-Percp-Cy5.5
CD3-PE-Cy7

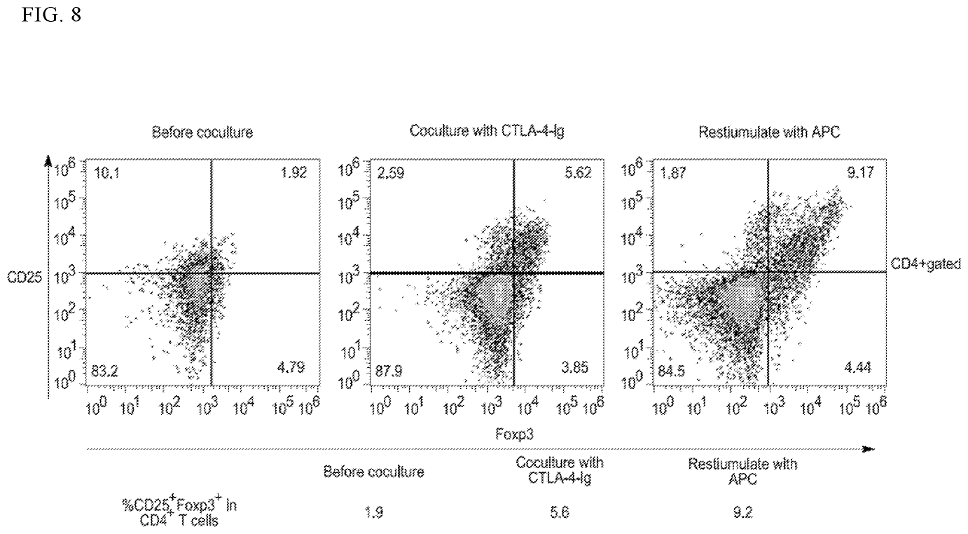

Results are shown in FIG. 8. Foxp3 expression was significantly increased when CD4+ T cells from donor were cocultured with CTLA-4-Ig and APC from patient (from 1.9% to 5.6%). Frequency of Foxp3+ would be further upregulated after removing CTLA-4-Ig and restimulating with APC from patient (from 5.6% to 9.2%).

Stimulation of CD4+CD25+CD127−/Lo Treg Cells

CD4+CD25+CD127−/lo Treg cells were stimulated using CD3/CD28/CD2 T cell activators at the ratio of 25 uL per 1 M cells. IL-2 was added into the flask. The final concentration was 500 IU/ml.

The Treg cells were cultured in 5% CO₂ at 37° C. in an incubator.

Day 8

For aTreg: The culturing volume was doubled with complete media and IL-2 (500 IU/ml). Fresh complete media with IL-2 was added every 2-3 days.

Day 10

For nTreg-D9: The culture was collected into a 250 ml tube. The flask was washed in 20 ml media and transferred into the same tube. The culture was spun down at 1200 rpm for 10 min. Supernatant was removed and the pellet was resuspended in 5 ml media. The cells were counted. The cells were resuspended with complete media with 500 IU/ml IL2. The cell concentration was 1M/ml and the cells were stimulated with T cell activator at ratio of 25 uL per 1 M cells. The flask was placed into the incubator.

Day 15

For aTreg-D9: The aTreg culture was collected into a 250 ml tube. The flask was washed in 20 ml media and transferred into the same tube. Cells were spun down at 1200 rpm for 10 min. Supernatant was removed and the pellet was resuspended in 5 ml media. The cells were counted. The cells were resuspended with complete media with 500 IU/ml IL2. The cell concentration was 1M/ml. The cells were stimulated T cell activator at a ratio of 25 uL per 1 M of cells. The flask was placed into incubator.

Day 16

PBMC were thawed and X-vivo+10% HAB serum was prepared.

| | Stock | Desired | Vol. |
|---|---|---|---|
| Component | conc. | conc. | (mL) |
| X-vivo 20 | 1× | 1× | 450 |
| Human AB | 100% | 10% | 50 |

One vial of PBMC cells from liquid nitrogen tank was thawed using warm X-vivo20 complete medium. The cells were spun down at 1200 rpm for 10 min at 20° C. The supernatant was removed. The cell pellet was resuspended with 25 ml X-vivo 20 complete medium. Cells were then counted.

The cell suspension was transferred into T175 and X-vivo 20 complete medium was used to wash the tube once. The wash suspension was transferred into flask. The cell concentration was 1-2 M cells/ml.

The flask was incubated at 5% CO2 at 37° C. in an incubator for overnight culturing.

Day 17

PBMC preparation: The contents of the flask were mixed and transferred into a 250 ml tube. The flask was washed and the rinse solution was combined into the tube. The tube was spun down at 1200 rpm for 10 minutes, supernatant was discarded and cells were resuspended in 30 ml. The cells were then counted and 10E6 cells were removed for suppression assay.

nTreg-D16 Harvest and distribution: nTreg cells were collected into 250 ml tubes. 15 ml of supernatant was saved after centrifuging for cytokine release assay. Each flask was washed with 20 ml of HBSS+0.2% HSA and combined with culture in the tubes. The tubes were spun down at 1200 RPM for 10 min. The supernatant was removed and the pellet was resuspended with complete media. The cells were then counted, and 2 M cells were used to perform the FACS analysis.

The FACS panel for unstained control and stained sample was as follows:

Foxp3 panel
Foxp3-BV421
CD8-Pacific blue
CD4-AF488
CD127-PE
CD19-BV605
CD25-APC
CD3-PE-Cy7

For the Foxp3-TSDR assay, 1M cells were frozen. For the suppression assay, 2M cells were used.

Expression analysis of nTreg cells isolated from donor PBMC that were previously frozen was completed and showed that yields between from 2.1% to 5.5% of highly pure cells were obtained from the donor PBMC cells.

nTreg-D16 In Vitro Suppression Assay Setup

Responder cell were PBMCs, Treg were expanded from nTreg-D16, medium used was X-vivo 20/10% AB serum, the stimulator was anti-CD3/28 dynabeads and the ratio of responder cells to beads was 1:1.

First, 10E6 PBMC were centrifuged at 1200 rpm for 10 min. The supernatant was then removed.

For CFSE labeling, PBMC were resuspended in 500 ul HBSS buffer and transferred into 1.5 ml eppendorf tube. Another 500 ul HBSS buffer was used to wash the original tube and the rinse buffer was transferred into the same eppendorf tube. The tube was spun down at 8000 rpm for 2 min at room temperature. Cells were resuspended with 500 ul prewarmed HBSS buffer and CFSE (The stock solution was 5 mM, and the final concentration was 10 uM) was added. The cells were incubated in 37° C. waterbath for 15 min. The dye was quenched with ice-cold medium containing 10% serum and spun down. Subsequently, X-vivo20 complete medium was used to was the cells one time. The cells were then resuspended with X-vivo20 complete medium.

The cells were counted and the cell concentration was adjusted to 1M/ml. The Treg cell concentration was adjusted to 1 M/ml. 0.1 M of the responder cells (PBMC) were seeded per well in a U-bottom 96 well plate with varying ratios of Treg cells as follows: Responders to Treg ratios were 1:1, 1:0.5, 1:0.25, 1:0.125, 1:0, and no stimulation.

Cells were stimulated with anti-CD3/anti-CD28 dynabeads. The ratio of responder cells to beads was 1:1. Then, 0.1 M beads were resuspended into 50 ul CM. 50 ul of dynabeads were removed and 950 ul of CM was added for 2 M cells/ml. 50 ul was added into each respective well. The final volume was 250 ul.

| | | | AntiCD3/28 | |
|---|---|---|---|---|
| | PBMC | Treg | bead | CM |
| | (ul) | (ul) | (ul) | (ul) |
| 1:1 | 100 | 100 | 50 | 0 |
| 01:0.5 | 100 | 50 | 50 | 50 |
| 01:0.25 | 100 | 25 | 50 | 75 |
| 01:0.125 | 100 | 12.5 | 50 | 87.5 |
| 1:0 | 100 | 0 | 50 | 100 |
| No stimulation | 100 | 0 | 0 | 150 |

Cultures were maintained for 3 days. CFSE+CD8+ cells were analyzed by FACS on Day 3.

Day 21

PBMC were thawed. X-vivo+10% HAB serum was prepared.

| | Stock | Desired | Vol. |
|---|---|---|---|
| Component | conc. | conc. | (mL) |
| X-vivo 20 | 1× | 1× | 450 |
| Human AB | 100% | 10% | 50 |

One vial of PBMC cells from liquid nitrogen tank was thawed using warm X-vivo20 complete medium. The cells were spun down at 1200 rpm for 10 min at 20° C. The supernatant was removed. Cell pellet was resuspended with 25 ml x-vivo 20 complete medium and the cells were counted. The cell suspension was transferred into T175 and X-vivo 20 complete medium was used to wash the tube once. The wash suspension was transferred into a flask. The cell concentration was 1-2 M/ml.

The flask was incubated at 5% CO₂ in a 37° C. incubator for overnight culturing.

Day 22—aTreg-D16 Harvest and Distribution

nTreg cells were collected into 250 ml tubes. After centrifuging for the cytokine release assay, 15 ml supernatant was saved. Each flask was washed with 20 ml of HBSS+0.2% HSA and combined with culture in the tubes. The tubes were spun down at 1200 RPM for 10 min. The supernatant was removed and pellet resuspended with complete media. Cells were counted.

2 M cells were used to perform the FACS analysis. The FACS panel for unstained control and stained sample was as follows:

Foxp3 panel
Foxp3-BV421
CD8-Pacific blue
CD4-AF488
CD127-PE
CD19-BV605
CD25-APC
CD3-PE-Cy7

1 M cells were frozen for Foxp3-TSDR assay. 2 M cells were taken out for the suppression assay.

aTreg-D16 in—Vitro Suppression Assay Setup

Responder cells were PBMC, Treg were expanded from aTreg D16, the medium was X-vivo 20/10% AB serum, and the stimulator was anti-CD3/28 dynabeads. The ratio of responder cells to beads was 1:1.

First, 10E6 PBMC were centrifuged at 1200 rpm for 10 min. The supernatant was then removed.

CFSE labeling was conducted as follows: PBMC were resuspended in 500 ul HBSS buffer and transferred into a 1.5 ml eppendorf tube. Another 500 ul of HBSS buffer was used to wash the original tube. The rinse solution was transferred into the same eppendorf tube. The cells were spun down at 8000 rpm for 2 min at room temperature. Cells were resuspended with 500 ul prewarmed HBSS buffer and CFSE (the stock solution had 5 mM and the final concentration was 10 uM) was added. The cells were incubated in a 37° C. waterbath for 15 min. Subsequently, the dye was quenched with ice-cold medium containing 10% serum and spun down. The cells were washed once with X-vivo20 complete medium and then were resuspended with X-vivo20 complete medium.

The cells were counted and the cell concentration was adjusted to 1M cells/ml. The Treg cell concentration was adjusted to 1 M cells/ml. Then, 0.1 M responder cells (PBMC) were seeded per well in a U-bottom 96 well plate with varying ratios of Treg cells. The responder to Treg ratios used were 1:1, 1:0.5, 1:0.25, 1:0.125, 1:0 and no stimulation.

Cells were stimulated with anti-CD3/anti-CD28 dynabeads. The ratio of responder cells to beads was 1:1. After, 0.1 M beads were resuspended into 50 ul CM. 50 ul of dynabeads were removed and 950 ul of CM was added to get 2 M cells/ml. Then 50 ul was added into the respective well. The total volume was 250 ul.

| | | | AntiCD3/28 | |
|---|---|---|---|---|
| | PBMC | Treg | bead | CM |
| | (ul) | (ul) | (ul) | (ul) |
| 1:1 | 100 | 100 | 50 | 0 |
| 01:0.5 | 100 | 50 | 50 | 50 |
| 01:0.25 | 100 | 25 | 50 | 75 |
| 01:0.125 | 100 | 12.5 | 50 | 87.5 |
| 1:0 | 100 | 0 | 50 | 100 |
| No stimulation | 100 | 0 | 0 | 150 |

Cultures were maintained for 3 days and CFSE+CD8+ cells were analyzed by FACS on Day 3.

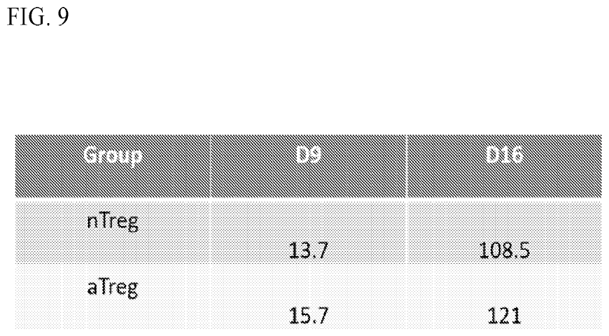

Results show the expansion of nTreg and aTreg at different time points in FIG. 9. Both nTreg and aTreg were expanded by more than 100 fold in vitro within the 16 day course. Further, nTreg from fresh and frozen PBMC may be expanded by more than 100 fold in vitro.

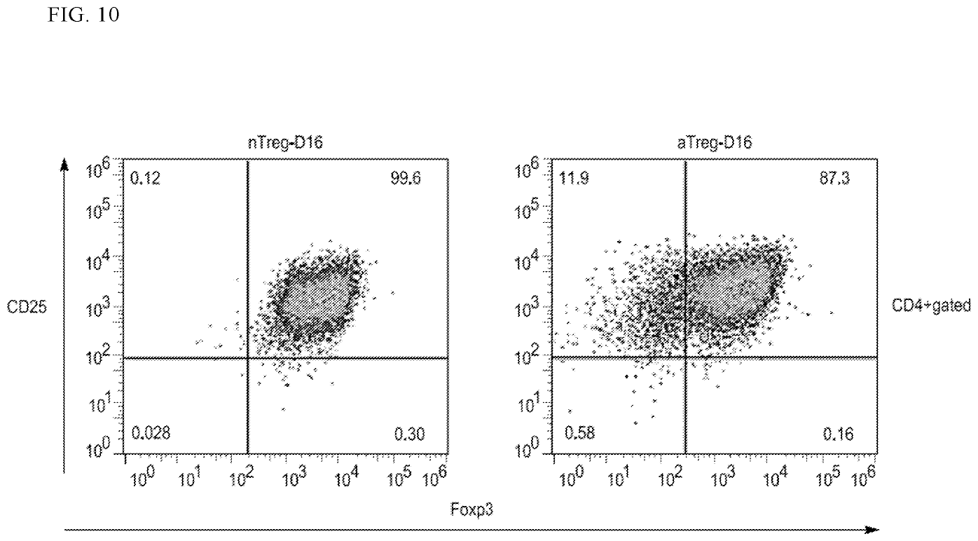

Results are shown for Foxp3 expression levels after expansion in FIG. 10. Expanded aTreg-D16 maintained similar levels of Foxp3 compared to nTreg on D16.

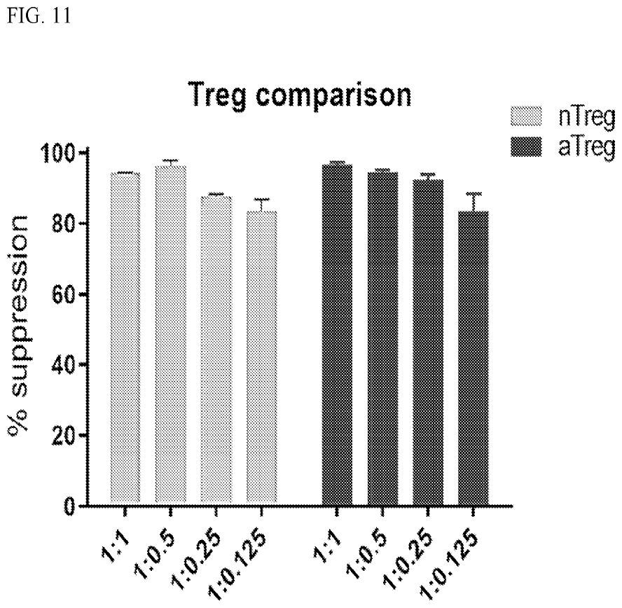

Results are shown for the comparison of Treg-D16 function in FIG. 11. Expanded aTreg-D16 showed a comparably suppressive capacity compared to nTreg.

Day 23-30

Protocol for Foxp3-TSDR Detection Assay

Genomic DNA isolation and bisulfite treatment was completed as follows: Genomic DNA (gDNA) was extracted from frozen nTreg cells and aTreg cells using the DNeasy blood and tissue kit (QIAGEN, Valencia, CA). The gDNA samples were treated using the EZ-DNA methylation-gold kit (Zymo Research, Orange, CA) according to the manufacturer's recommendations.

Droplet digital methylation-specific PCR assay (ddMSP) was completed as follows: Approximately 10-50 ng of bisulfite-treated DNA were used per sample for ddPCR reaction. VIC fluorescent-tagged TaqMan assay probe was used to quantify the copy number of the unmethylated TSDR of FOXP3 of Treg. FAM fluorescent-tagged TaqMan assay probe was used quantify the methylated TSDR of FOXP3 of Treg cells. The ddPCR system was operated according to the manufacturer's instructions. The PCR reaction solution was dispensed into a single well on a 96-well plate already containing ddPCR Supermix for Probes (no dUTP) (Bio-Rad), 900 nmol/L primers, and 250 nmol/L probe in a final volume of 25 μl. Primer and probe sequences used are listed (Table 2).

TABLE 2

| | | | SEQ |
|---|---|---|---|
| Primers and probes for qMSP and qBSP | | | ID NO |
| P17 | UMTreg-For | 5′-GTATTTGGGTTTTGTTGTTATAGTTTTT-3′ | 1 |
| P18 | UMTreg-Rev | 5′-CTACAAAACAAAACAACCAATTCTCA-3′ | 2 |
| P19 | MTreg-For | 5′-GTATTTGGGTTTTGTTGTTATAGTTTTC-3′ | 3 |
| P20 | MTreg-Rev | 5′-TACAAAACAAAACAACCAATTCTCG-3′ | 4 |
| P24 | MTreg-Probe with | 5′-CGACGCATCCGACCGCCA-3′ | 5 |
| | FAM | | |
| P25 | UMTreg-Probe | 5′-ACCCAACACATCCAACCACCA-3′ | 6 |
| | with VIC | | |
| P26 | BSTSDR-Treg-For | 5′-GTTTGTATTTGGGTTTTGTTGTTATAG-3′ | 7 |
| P27 | BSTSDR_Treg- | 5′-CTACTACAAAACAAAACAACCAATTC-3′ | 8 |
| | Rev | | |
| P31 | BSTSDR-Probe | 5′-ATCTACCCTCTTCTCTTCCTC-3′ | 9 |
| | with FAM | | |

Droplets were generated using 20 μl of the assay mix and 70 μl of droplet generation oil into the QX200 DG cartridge (Bio-Rad), then loaded into the QX200 Droplet Generator (Bio-Rad). The droplets were transferred into a 96-well PCR plate and the plate was heat sealed with foil using a PX1 Plate Sealer (Bio-Rad). PCR reactions were performed using the C100 Touch thermal cycler (Bio-Rad) with the following PCR conditions: a 10 min enzyme activation at 95° C. followed by 40 cycles of denaturation at 94° C. for 30 s and annealing-extension at 55° C. for 1 min. An enzymatic-deactivation step was included at the end at 98° C. for 10 min and plates were stored at 10° C. until droplets were counted using the Bio-Rad QX200 Droplet Reader (Bio-Rad). The droplet reader was used to count the number of droplets that were positive and negative for FAM and VIC fluorophore. ddPCR was run in triplicates. No-template controls were included in each run as negative controls to control for contamination during reactions. Analysis of the ddPCR data was performed with QuantaSoft analysis software (Bio-Rad) that accompanied the QX200 Droplet Reader.

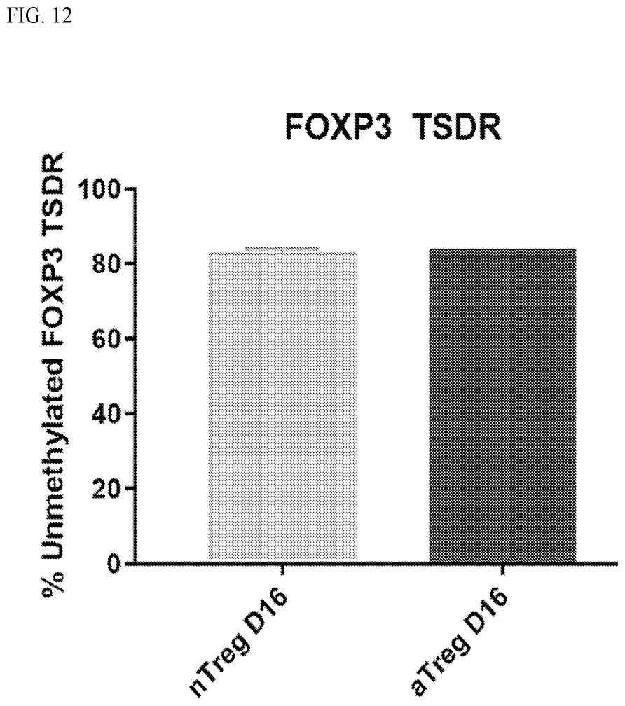

A comparison of Foxp3-TSDR of different Treg cells on D16 is shown in FIG. 12. Expanded aTreg on D16 showed a similar level of Foxp3-TSDR (Treg-specific demethylated region) compared to nTreg. (TSDR is non-coding element in Foxp3 gene required for stable expression).

Cytokine Profile Analysis of Different Treg Cells

A total of 6 cytokines (IL-4, IL-6, IL-10, IL-17A, IFN-g and TNF-α) were analyzed by using LEGENDplex kit from Biolegend.

The individual beads (13×) were mixed with each other and diluted to 1× final concentration with Assay Buffer prior to use. The steps as follows were used to mix the beads (a 5-plex subpanel is described below):

Each bead vial was sonicated for 1 minute in a sonicator bath and then vortexed for 30 seconds to resuspend the beads. The amount of mixed and diluted beads needed for the assay was calculated. An excess of beads were prepared to compensate for pipetting loss. Each reaction required 25 μL of mixed and diluted beads. For 50 reactions, 1.5 mL of mixed beads was prepared. For 100 reactions, 3 mL of mixed beads was prepared. For 1.5 ml of 5-plex 1× diluted beads, 115 μL of each of the 5 individual beads (13×) were transferred to a fresh tube (total bead volume=575 μL) and 925 μL of Assay Buffer was added to make the final volume of 1.5 mL.

Preparation of Wash Buffer was as follows: The 20× Wash Buffer was allowed to equilibrate to room temperature, and was mixed to bring all salts into solution. Afterwards, 25 mL of 20× Wash Buffer was diluted with 475 mL deionized water. Unused portions were stored between 2° C. and 8° C. for up to one month.

Preparation of standards was completed as follows: Prior to use, the lyophilized Human Th Panel Standard Cocktail was reconstituted with 250 μL Assay Buffer. The vial was mixed and allowed to sit at room temperature for 10 minutes. The standard was then transferred to an appropriately labeled polypropylene microcentrifuge tube. This was used as the top standard C7. Six polypropylene microcentrifuge tubes were labeled as C6, C5, C4, C3, C2 and C1, respectively. 75 μL of Assay Buffer was added to each of the six tubes. 1:4 dilutions were prepared of the top standard by transferring 25 μL of the top standard C7 to the C6 tube and mixing well. This was the C6 standard. In the same manner, serial 1:4 dilutions were performed to obtain C5, C4, C3, C2 and C1 standards (see the table below using the top standard at 10,000 pg/mL as an example). Assay Buffer was used as the 0 pg/mL standard (CO).

| Tube/ | | Assay Buffer | | Final |
|---|---|---|---|---|
| Standard | Serial | to add | Standard | Conc. |
| ID | Dilution | (ul) | to add | (pg/ml) |
| C7 | — | — | — | 10,000 |
| C6 | | 75 | 25 μL of C7 | 2,500 |
| C5 | 1:16 | 75 | 25 μL of C6 | 625 |
| C4 | 1:64 | 75 | 25 μL of C5 | 156.3 |
| C3 | 1:256 | 75 | 25 μL of C4 | 38.1 |
| C2 | 1:1024 | 75 | 25 μL of C3 | 9.8 |
| C1 | 1:4096 | 75 | 25 μL of C2 | 2.4 |
| C0 | — | 75 | — | 0 |

Performing Assay Using a V-Bottom Plate

All reagents were allowed to warm to room temperature (20-25° C.) before use. The plate was kept upright during the entire assay procedure, including the washing steps, to avoid losing beads. The plate was placed in the dark or wrapped with aluminum foil for all incubation steps. Standards and samples were run in duplicate and arranged on the plate in a vertical configuration convenient for data acquisition and analysis. Standards were loaded in the first two columns.

For measuring cell culture supernatant samples, the plate was loaded as shown in the table below (in the order from left to right):

| | Assay | | | |
|---|---|---|---|---|
| | Buffer | Matrix B | Standard | Sample* |
| Standard Wells | 25 μL | — | 25 μL | — |
| Sample wells | 25 μL | — | — | 25 μL |

Mixed beads were vortexed for 30 seconds. 25 μL of mixed beads were added to each well. The total volume was 75 μL in each well after beads addition. It is noted that during addition of the beads, the mixed beads bottle was agitated intermittently to avoid bead settling.

The plate was sealed with a plate sealer. The entire plate was covered with aluminum foil to protect the plate from light. The plate was shaken on a plate shaker at 800 rpm, or at an optimal speed that is high enough to keep beads in suspension during incubation, but not too high so it causes spill from the wells. The plate was shaken for 2 hours at room temperature.

The plate was centrifuged at 1050 rpm (˜250 g) for 5 minutes, using a swinging bucket rotor (G.H 3.8) with microplate adaptor. The supernatant was removed by quickly inverting and flicking the plate in one continuous and forceful motion. The plate was blotted and the remaining liquid was drained from the wells without disturbing the bead pellet. The plate was washed by dispensing 200 μL of 1× Wash Buffer into each well and incubating for one minute. The centrifugation and supernatant removal were repeated. A second wash was optional, but helped reduce background.

Detection Antibodies at a volume of 25 μL was added to each well. The plate was sealed with a new plate sealer. The entire plate was covered with aluminum foil to protect the plate from light. The plate was shaken at 800 rpm on a plate shaker for 1 hour at room temperature. Without washing the plate 25 μL of SA-PE was directed added to each well. The plate was sealed with a new plate sealer. The entire plate was wrapped with aluminum foil and the plate was shaken on a plate shaker at approximate 800 rpm for 30 minutes at room temperature.

The plate was centrifuged at 1050 rpm (˜250 g) for 5 minutes, using a swinging bucket rotor (G.H 3.8) with microplate adaptor. The supernatant was removed by quickly inverting and flicking the plate in one continuous and forceful motion. The plate was blotted and the remaining liquid was drained from the wells without disturbing the bead pellet. The plate was washed by dispensing 200 μL of 1× Wash Buffer into each well and incubating for one minute. The centrifugation and supernatant removal was repeated. This washing step was optional but helped to reduce the background.

Subsequently, 150 μL of 1× Wash Buffer was added to each well, and the beads were resuspended by pipetting. Samples were read on a flow cytometer, preferably within the same day of the assay since prolonged sample storage may lead to reduced signal.

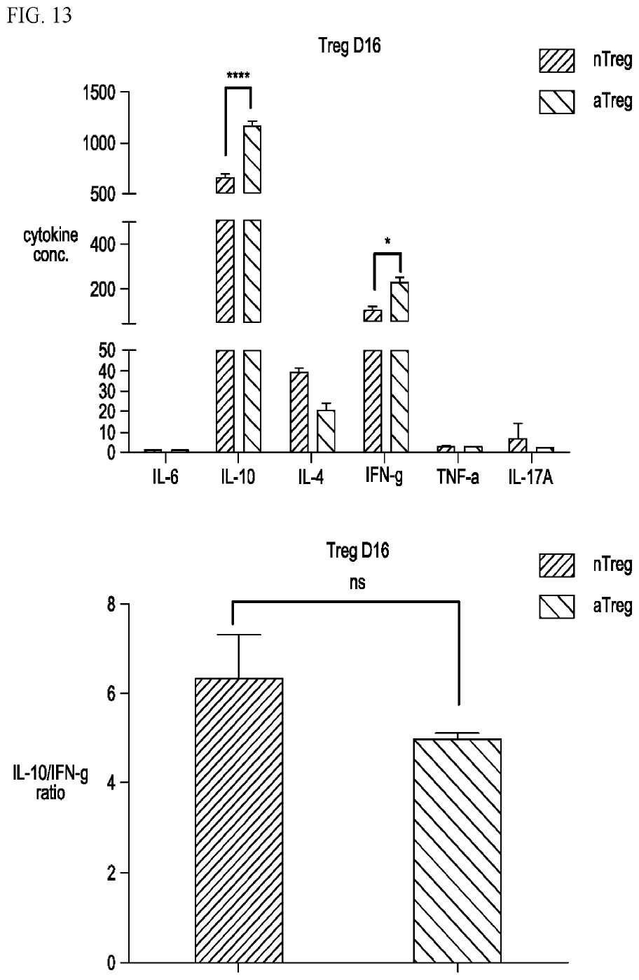

Cytokine profiles of different Treg cells after expansion are shown in FIG. 13. Expanded aTreg on D16 produced more IL-10, IFN-g than nTreg, but the ratio of IL10/IFN-g was not significantly different.

What is claimed is:

1. A method of treating or preventing graft-versus-host disease in a subject in need thereof, the method comprising administering to the subject a therapeutically effective amount of recipient antigen-specific regulatory T cells, thereby treating or preventing graft-versus-host disease in said subject;
wherein said recipient antigen-specific regulatory T cells are derived from regulatory T cells from a donor of a tissue transplant to said subject; and
wherein said tissue transplant is a cause of graft-versus-host disease in said subject.

2. The method of claim 1, wherein said regulatory T cells are obtained from said donor within one week said tissue transplant was obtained from said donor.

3. The method of claim 1, wherein said regulatory T cells are isolated from said donor's blood.

4. The method of claim 1, wherein said tissue transplant comprises hematopoietic stem cells.

5. The method of claim 1, wherein said subject has or previously had cancer.

6. The method of claim 5, wherein said cancer is leukemia, lymphoma, sarcoma, myeloma, or glioma.

7. The method of claim 1, wherein said recipient antigen-specific regulatory T cells are formed by a method comprising:
(a) expanding said regulatory T cells in vitro, thereby forming a plurality of regulatory T cells; and
(b) contacting said plurality of regulatory T cells with a plurality of recipient antigen presenting cells and a CD28 inhibitor compound in vitro, thereby forming said recipient antigen-specific regulatory T cells.

8. The method of claim 7, wherein step (b) further comprises contacting said plurality of regulatory T cells with a second plurality of recipient antigen presenting cells.

9. The method of claim 7, wherein said plurality of recipient antigen presenting cells are taken from said subject's blood.

10. The method of claim 7, wherein said plurality of recipient antigen presenting cells are taken from said subject's tissue transplant.

11. The method of claim 7, wherein said plurality of recipient antigen presenting cells are taken from said subject when the subject has graft-versus-host disease.

12. The method of claim 7, wherein said plurality of recipient antigen presenting cells are taken from said subject prior to administration of a graft-versus-host disease therapeutic treatment.

13. The method of claim 12, wherein said graft-versus-host disease therapeutic treatment comprises a corticosteroid and/or immunosuppressive compound.

14. The method of claim 13, wherein said immunosuppressive compound is Methotrexate, Cyclosporine, Tacrolimus, Mycophenolate mofetil, Sirolimus, Antithymocyte globulin, Alemtuzumab, Cyclophosphamide or Inolimomab.

15. The method of claim 14, wherein said corticosteroid is prednisone, methylprednisolone, dexamethasone, beclomethasone or budesonide.

16. The method of claim 7, wherein said plurality of recipient antigen presenting cells comprise dendritic cells.

17. The method of claim 7, wherein said plurality of recipient antigen presenting cells comprise B cells.

18. The method of claim 7, wherein said CD28 inhibitor compound is a CD80/CD86 antagonist.

19. The method of claim 18, wherein the CD28 inhibitor compound is abatacept or belatacept.

20. The method of claim 1, wherein said recipient antigen-specific regulatory T cells are formed by a method comprising:
(a) expanding regulatory T cells in vitro, thereby forming a plurality of regulatory T cells;
(b) contacting a plurality of recipient antigen presenting cells with recipient antigen in vitro, thereby forming a plurality of activated recipient antigen presenting cells; and
(c) contacting said plurality of regulatory T cells with said plurality of activated recipient antigen presenting cells in the presence of a CD28 inhibitor compound, thereby forming said recipient antigen-specific regulatory T cells.

21. The method of claim 20, wherein step (c) further comprises contacting said plurality of regulatory T cells with a second plurality of activated recipient antigen presenting cells.

22. The method of claim 20, wherein said plurality of recipient antigen presenting cells are taken from said subject's blood.

23. The method of claim 20, wherein said plurality of recipient antigen presenting cells are taken from said subject's tissue transplant.

24. The method of claim 20, wherein said plurality of recipient antigen presenting cells are taken from said subject when the subject has graft-versus-host disease.

25. The method of claim 20, wherein said plurality of recipient antigen presenting cells are taken from said subject prior to administration of a graft-versus-host disease therapeutic treatment.

26. The method of claim 25, wherein said graft-versus-host disease therapeutic treatment comprises a corticosteroid and/or immunosuppressive compound.

27. The method of claim 26, wherein said immunosuppressive compound is Methotrexate, Cyclosporine, Tacrolimus, Mycophenolate mofetil, Sirolimus, Antithymocyte globulin, Alemtuzumab, Cyclophosphamide or Inolimomab.

28. The method of claim 26, wherein said corticosteroid is prednisone, methylprednisolone, dexamethasone, beclomethasone or budesonide.

29. The method of claim 20, wherein said plurality of recipient antigen presenting cells comprise dendritic cells.

30. The method of claim 20, wherein said plurality of recipient antigen presenting cells comprise B cells.

31. The method of claim 20, wherein the CD28 inhibitor compound is a CD80/CD86 antagonist.

32. The method of claim 31, wherein the CD28 inhibitor compound is abatacept or belatacept.

33. A method of forming recipient antigen-specific regulatory T cells, the method comprising:
(a) expanding regulatory T cells in vitro, wherein said regulatory T cells are from a donor of a tissue transplant, thereby forming a plurality of regulatory T cells; and
(b) contacting said plurality of regulatory T cells with a plurality of recipient antigen presenting cells and a CD28 inhibitor compound in vitro, wherein said plurality of recipient antigen presenting cells is from a subject who has received said tissue transplant, thereby forming said recipient antigen-specific regulatory T cells.

34. The method of claim 33, wherein step (b) further comprises contacting said plurality of regulatory T cells with a second plurality of recipient antigen presenting cells.

35. The method of claim 33, wherein said plurality of recipient antigen presenting cells is taken from said subject's blood.

36. The method of claim 33, wherein said plurality of recipient antigen presenting cells is taken from said subject's tissue transplant.

37. The method of claim 33, wherein said plurality of recipient antigen presenting cells is taken from said subject when the subject has graft-versus-host disease.

38. The method of claim 33, wherein said plurality of recipient antigen presenting cells is taken from said subject prior to administration of a graft-versus-host disease therapeutic treatment.

39. The method of claim 38, wherein said graft-versus-host disease therapeutic treatment comprises a corticosteroid and/or immunosuppressive compound.

40. The method of claim 39, wherein said immunosuppressive compound is Methotrexate, Cyclosporine, Tacrolimus, Mycophenolate mofetil, Sirolimus, Antithymocyte globulin, Alemtuzumab, Cyclophosphamide or Inolimomab.

41. The method of claim 39, wherein said corticosteroid is prednisone, methylprednisolone, dexamethasone, beclomethasone or budesonide.

42. The method of claim 33, wherein said plurality of recipient antigen presenting cells comprise dendritic cells.

43. The method of claim 33, wherein said plurality of recipient antigen presenting cells comprise B cells.

44. The method of claim 33, wherein said CD28 inhibitor compound is a CD80/CD86 antagonist.

45. The method of claim 44, wherein the CD28 inhibitor compound is abatacept or belatacept.

46. A method of forming recipient antigen-specific regulatory T cells, the method comprising:
(a) expanding regulatory T cells in vitro, wherein said regulatory T cells are from a donor of a tissue transplant, thereby forming a plurality of regulatory T cells;
(b) contacting a plurality of recipient antigen presenting cells with a recipient antigen in vitro, wherein said plurality of recipient antigen presenting cells is from a subject who has received said tissue transplant, thereby forming a plurality of activated recipient antigen presenting cells; and
(c) contacting said plurality of regulatory T cells with said plurality of activated recipient antigen presenting cells and a CD28 inhibitor compound in vitro, thereby forming said recipient antigen-specific regulatory T cells.

47. The method of claim 46, wherein step (c) further comprises contacting said plurality of regulatory T cells with a second plurality of activated recipient antigen presenting cells.

48. The method of claim 46, wherein said plurality of recipient antigen presenting cells are taken from said subject's blood.

49. The method of claim 46, wherein said recipient antigen presenting cells are taken from said subject's tissue transplant.

50. The method of claim 46, wherein said recipient antigen presenting cells are taken from said subject prior to administration of a graft-versus-host disease therapeutic treatment.

51. The method of claim 50, wherein said graft-versus-host disease therapeutic treatment comprises a corticosteroid and/or immunosuppressive compound.

52. The method of claim 51, wherein said immunosuppressive compound is Methotrexate, Cyclosporine, Tacrolimus, Mycophenolate mofetil, Sirolimus, Antithymocyte globulin, Alemtuzumab, Cyclophosphamide or Inolimomab.

53. The method of claim 51, wherein said corticosteroid is prednisone, methylprednisolone, dexamethasone, beclomethasone or budesonide.

54. The method of claim 46, wherein said plurality of recipient antigen presenting cells comprise dendritic cells.

55. The method of claim 46, wherein said plurality of recipient antigen presenting cells comprise B cells.

56. The method of claim 46, wherein said CD28 inhibitor compound is a CD80/CD86 antagonist.

57. The method of claim 56, wherein the CD28 inhibitor compound is abatacept or belatacept.

58. An antigen-specific regulatory T cell derived from a donor of a transplant tissue, wherein said antigen-specific regulatory T cell specifically comprises a T-cell receptor that specifically binds a transplant tissue antigen from a recipient of said transplant tissue.

59. A pharmaceutical composition comprising recipient antigen-specific regulatory T cells, wherein said recipient antigen-specific regulatory T cells are formed by the method of claim 33.


SEQUENCE LISTING (9 sequences)

SEQ ID NO: 1 - DNA, 28 bp, Artificial Sequence
  Synthetic polynucleotide
gtatttgggttttgttgttatagttttt

SEQ ID NO: 2 - DNA, 26 bp, Artificial Sequence
  Synthetic polynucleotide
ctacaaaacaaaacaaccaattctca

SEQ ID NO: 3 - DNA, 28 bp, Artificial Sequence
  Synthetic polynucleotide
gtatttgggttttgttgttatagttttc

SEQ ID NO: 4 - DNA, 25 bp, Artificial Sequence
  Synthetic polynucleotide
tacaaaacaaaacaaccaattctcg

SEQ ID NO: 5 - DNA, 18 bp, Artificial Sequence
  Synthetic polynucleotide
cgacgcatccgaccgcca

SEQ ID NO: 6 - DNA, 21 bp, Artificial Sequence
  Synthetic polynucleotide
acccaacacatccaaccacca

SEQ ID NO: 7 - DNA, 27 bp, Artificial Sequence
  Synthetic polynucleotide
gtttgtatttgggttttgttgttatag

SEQ ID NO: 8 - DNA, 26 bp, Artificial Sequence
  Synthetic polynucleotide
ctactacaaaacaaaacaaccaattc

SEQ ID NO: 9 - DNA, 21 bp, Artificial Sequence
  Synthetic polynucleotide
atctaccctcttctcttcctc